(19) United States
(12) Patent Application Publication
(10) Pub. No.: US 2023/0286929 A1
(43) Pub. Date: Sep. 14, 2023

(54) AROMATIC BRIDGED RING AMIDE DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/319,920

(22) Filed: May 18, 2023

Related U.S. Application Data
(63) Continuation of application No. PCT/EP2021/082590, filed on Nov. 23, 2021.

(30) Foreign Application Priority Data
Nov. 25, 2020 (WO) PCT/CN2020/131372

(51) Int. Cl. C07D 263/54; A61P 31/20

(52) U.S. Cl. CPC C07D 263/54; A61P 31/20

(57) ABSTRACT

The present invention provides novel compounds having the general formula:

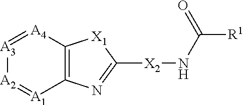

wherein A₁ to A₄, X₁, X₂ and R¹ are as described herein, compositions including the compounds and methods of using the compounds.

### CROSS-REFERENCE TO PRIOR APPLICATIONS

This application is a continuation of International Application No. PCT/EP2021/082590 having an International Filing Date of Nov. 23, 2021 and which claims benefit under 35 U.S.C. § 119 to International Application No. PCT/CN2020/131372 having an International Filing Date of Nov. 25, 2020. The entire contents of both are incorporated herein by reference.

### FIELD OF THE INVENTION

The present invention relates to aromatic bridged ring amide derivatives having pharmaceutical activity of formula (I) useful for therapy and/or prophylaxis of HBV infection in a mammal, and in particular to HBsAg (HBV Surface antigen) and HBeAg (HBV e antigen) inhibitors useful for treating HBV infection as well as their manufacture and pharmaceutical compositions containing them.

The present invention relates to compounds of formula (I)

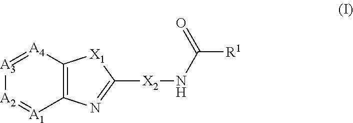

wherein A₁ to A₄, X₁, X₂, and R¹ are as described below, or a pharmaceutically acceptable salt thereof.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693). Moreover HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in supressing HBV DNA, but are not effective in reducing HBsAg level. Nucleos(t)ide analogs, even with prolonged therapy, have demonstrated HBsAg clearance rates comparable to those observed naturally (Janssen et al. Lancet, (2005), 365, 123-129; Marcellin et al. N. Engl. J. Med., (2004), 351, 1206-1217; Buster et al. Hepatology, (2007), 46, 388-394). Therefore, there is an urgent need for the development of novel therapeutic agents that could efficiently reduce HBsAg. (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

The present invention relates to a compound of formula (I)

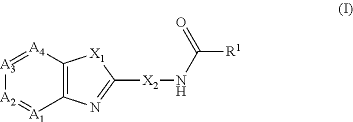

wherein
R¹ is selected from heterocyclyl, heterocyclylC₁₋₆alkyl and phenyl; wherein heterocyclyl, heterocyclylC₁₋₆alkyl and phenyl are unsubstituted or substituted by one or two or three substituents independently selected from halogen, C₁₋₆alkyl, haloC₁₋₆alkyl, C₃₋₇cycloalkyl, heterocyclyl, heterocyclylC₁₋₆alkyl, C₁₋₆alkylsulfonyl, C₁₋₆alkylsulfinyl, C₁₋₆alkylsulfanyl, C₃₋₇cycloalkylsulfinyl, C₃₋₇cycloalkylsulfonyl, C₃₋₇cycloalkylC₁₋₆alkylsulfinyl, C₃₋₇cycloalkylC₁₋₆alkylsulfonyl, aminosulfonyl, C₃₋₇cycloalkylsulfonimidoyl, C₁. 6alkylsulfonimidoyl, C₃-7cycloalkylC₁₋₆alkylsulfonimidoyl, heterocyclylC₁₋₆alkylsulfonyl, heterocyclylC₁₋₆alkylsulfonimidoyl, C₁₋₆alkylsulfonylC₁₋₆alkyl, C₁₋₆alkylsulfonimidoylC₁₋₆alkyl, aminosulfonylC₁₋₆alkyl and C₁₋₆alkylcarbonylaminosulfonyl;
A₁ is selected from N and CR²; wherein R² is selected from H and halogen;
A₂ is selected from N and CR³; wherein R³ is selected from H and halogen;
A₃ is selected from N and CR⁴; wherein R⁴ is selected from H and halogen;
A₄ is selected from N and CR⁵; wherein R⁵ is selected from H and halogen;
X₁ is O;
X₂ is selected from

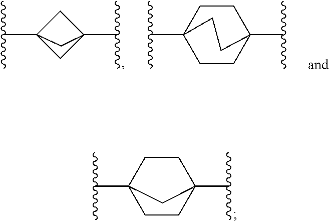

or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl. Most particular “C₁₋₆alkyl” group is methyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy and propoxy.

The term “C₃₋₇cycloalkyl” denotes to a saturated carbon mono or bicyclic ring or a saturated spiro-linked bicyclic carbon ring or a bridged carbon ring, containing from 3, 4, 5, 6, or 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋₇ cycloalkyl” group is cyclopropyl, cyclobutyl or cyclohexyl. Most particular “C₃₋₇cycloalkyl” group is cyclopropyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Examplary heterocyclyls are furyl, thienyl, pyridyl, pyrimidinyl, pyridazinyl, 2,2-dioxo-2lambda6-thiaspiro[3.3]heptanyl, 1,1-dioxothiolanyl, 1,1-dioxothianyl, 1,1-dioxothiolanylmethyl, 1,1-dioxothianylmethyl, 1,1-dioxothiazinanylmethyl and oxetanyl. Heterocyclyl may be optionally substituted by halogen, OH, SH, cyano, NH₂, NHCH₃, N(CH₃)₂, NO₂, N₃, C(O)CH₃, COOH, CO₂CH₃, C₁₋₆alkyl, C₁₋₆alkoxy, oxo, haloC₁₋₆alkyl, phenyl or heterocyclyl.

The term “carbonyl” alone or in combination refers to the group —C(O)—.

The term “sulfanyl” alone or in combination refers to the group —S—.

The term “sulfinyl” alone or in combination refers to the group —S(O)—.

The term “sulfonyl” alone or in combination refers to the group —S(O)₂—.

The term “sulfonimidoyl” alone or in combination refers to the group —S(O)(NH)—, whose formula is

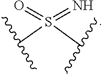 [SMILES: *S(*)(=N)=O]

The term “bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “oxo” refers to an ═O group and may be attached to a carbon atom or a sulfur atom.

As used herein, the wavy line “ [symbol] ” that intersects a bond in a chemical structure refers to the point of attachment of the bond to which the wavy bond intersects in the chemical structure fragment to the remainder of a molecule or structural formula.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

Compounds of the general formula (I) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

### HBV Inhibitors

The present invention provides (i) a compound having the general formula (I):

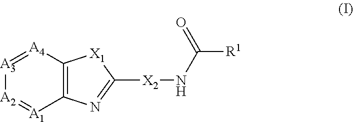

wherein
R¹ is selected from heterocyclyl, heterocyclylC₁₋₆alkyl and phenyl; wherein heterocyclyl, heterocyclylC₁₋₆alkyl and phenyl are unsubstituted or substituted by one or two or three substituents independently selected from halogen, C₁₋₆alkyl, haloC₁₋₆alkyl, C₃₋₇cycloalkyl, heterocyclyl, heterocyclylC₁₋₆alkyl, C₁₋₆alkylsulfonyl, C₁₋₆alkylsulfinyl, C₁₋₆alkylsulfanyl, C₃₋₇cycloalkylsulfinyl, C₃₋₇cycloalkylsulfonyl, C₃₋₇cycloalkylC₁₋₆alkylsulfinyl, C₃₋₇cycloalkylC₁₋₆alkylsulfonyl, aminosulfonyl, C₃₋₇cycloalkylsulfonimidoyl, C₁₋₆ alkylsulfonimidoyl, C₃₋₇cycloalkylC₁₋₆alkylsulfonimidoyl, heterocyclylC₁₋₆alkylsulfonyl, heterocyclylC₁₋₆alkylsulfonimidoyl, C₁₋₆alkylsulfonylC₁₋₆alkyl, C₁₋₆alkylsulfonimidoylC₁₋₆alkyl, aminosulfonylC₁₋₆alkyl and C₁₋₆alkylcarbonylaminosulfonyl;
A₁ is selected from N and CR²; wherein R² is selected from H and halogen;
A₂ is selected from N and CR³; wherein R³ is selected from H and halogen;
A₃ is selected from N and CR⁴; wherein R⁴ is selected from H and halogen;
A₄ is selected from N and CR⁵; wherein R⁵ is selected from H and halogen;
X₁ is O;
X₂ is selected from

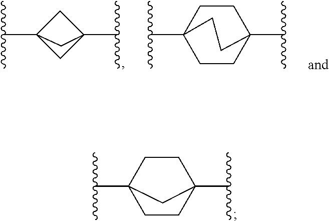

or a pharmaceutically acceptable salt thereof.

A further embodiment of the present invention is (ii) a compound of formula (I) according to (i), wherein
R¹ is selected from furyl, thienyl, pyridyl, pyrimidinyl, pyridazinyl, 2,2-dioxo-2lambda6-thiaspiro[3.3]heptanyl, 1,1-dioxothiolanyl, 1,1-dioxothianyl, 1,1-dioxothiolanylC₁₋₆alkyl, 1,1-dioxothianylC₁₋₆alkyl, 1,1-dioxothiazinanylC₁₋₆alkyl and phenyl; wherein furyl, thienyl, pyridyl, pyrimidinyl, pyridazinyl, 1,1-dioxothiolanyl and phenyl are unsubstituted or substituted by one or two or three substituents independently selected from halogen, C₁₋₆ alkyl, haloC₁₋₆alkyl, C₁₋₆alkylsulfonyl, C₁₋₆alkylsulfinyl, C₁₋₆alkylsulfanyl, C₃₋₇cycloalkylsulfinyl, C₃₋₇cycloalkylsulfonyl, C₃₋₇cycloalkylC₁₋₆alkylsulfinyl, C₃₋₇cycloalkylC₁₋₆alkylsulfonyl, aminosulfonyl, C₃₋₇cycloalkylsulfonimidoyl, C₁₋₆alkylsulfonimidoyl, C₃₋₇cycloalkylC₁₋₆alkylsulfonimidoyl, oxetanylC₁₋₆alkylsulfonyl, oxetanylC₁₋₆alkylsulfonimidoyl, C₁₋₆alkylsulfonylC₁₋₆alkyl, C₁₋₆alkylsulfonimidoylC₁₋₆alkyl, aminosulfonylC₁₋₆alkyl and C₁₋₆alkylcarbonylaminosulfonyl;
A₁ is CH;
A₂ is selected from N and CR³; wherein R³ is selected from H and halogen;
A₃ is CR⁴; wherein R⁴ is selected from H and halogen;
A₄ is selected from N and CR⁵; wherein R⁵ is selected from H and halogen;
X₁ is O;
X₂ is selected from

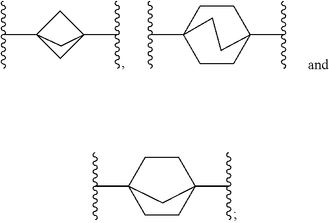

or a pharmaceutically acceptable salt thereof.

A further embodiment of the present invention is (iii) a compound of formula (I) according to (i), wherein
R¹ is selected from furyl, thienyl, pyridyl, pyrimidinyl, pyridazinyl, 2,2-dioxo-2lambda6-thiaspiro[3.3]heptanyl, 1,1-dioxothiolanyl, 1,1-dioxothianyl, 1,1-dioxothiolanylmethyl, 1,1-dioxothianylmethyl, 1,1-dioxothiazinanylmethyl and phenyl; wherein furyl, pyridyl, pyrimidinyl, pyridazinyl, 1,1-dioxothiolanyl and phenyl are unsubstituted or substituted by one or two or three substituents independently selected from Cl, Br, methyl, CF₃, methylsulfonyl, methylsulfinyl, methylsulfanyl, cyclopropylsulfinyl, cyclopropylsulfonyl, cyclopropylmethylsulfinyl, cyclopropylmethylsulfonyl, aminosulfonyl, cyclopropylsulfonimidoyl, methylsulfonimidoyl, cyclopropylmethylsulfonimidoyl, oxetanylmethylsulfonyl, oxetanylmethylsulfonimidoyl, methylsulfonylmethyl, methylsulfonimidoylmethyl, aminosulfonylmethyl and propylcarbonylaminosulfonyl;
A₁ is CH;
A₂ is selected from N and CR³; wherein R³ is selected from H and C₁;
A₃ is CR⁴; wherein R⁴ is selected from H, F and C₁;
A₄ is selected from N and CR⁵; wherein R⁵ is selected from H and C₁;
X₁ is O;
X₂ is selected from and

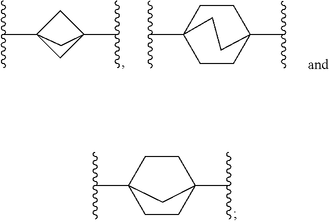

or a pharmaceutically acceptable salt thereof.

A further embodiment of the present invention is (iv) a compound of formula (I) according to (i), or a pharmaceutically acceptable salt thereof, wherein R¹ is furyl; wherein furyl is substituted by one substituent selected from C₃₋₇cycloalkylsulfonyl, C₃₋₇cycloalkylC₁₋₆ alkylsulfonyl, C₃₋₇cycloalkylsulfonimidoyl, C₁₋₆alkylsulfonimidoyl, C₃₋₇cycloalkylC₁₋₆alkylsulfonimidoyl and C₁₋₆alkylsulfonylC₁₋₆alkyl.

A further embodiment of the present invention is (v) a compound of formula (I) according to (i), or a pharmaceutically acceptable salt thereof, wherein R¹ is furyl; wherein furyl is substituted by one substituent selected from cyclopropylsulfonyl, cyclopropylmethylsulfonyl, cyclopropylsulfonimidoyl, methylsulfonimidoyl, cyclopropylmethylsulfonimidoyl and methylsulfonylmethyl.

A further embodiment of the present invention is (vi) a compound of formula (I) according to (i), or a pharmaceutically acceptable salt thereof, wherein A₂ is CR³; wherein R³ is selected from H and halogen.

A further embodiment of the present invention is (vii) a compound of formula (I) according to (i), or a pharmaceutically acceptable salt thereof, wherein R³ is selected from H and Cl.

A further embodiment of the present invention is (viii) a compound of formula (I) according to (i), or a pharmaceutically acceptable salt thereof, wherein A₃ is CR⁴; wherein R⁴ is selected from H and halogen.

A further embodiment of the present invention is (ix) a compound of formula (I) according to (viii), or a pharmaceutically acceptable salt thereof, wherein R⁴ is selected from H and C₁.

A further embodiment of the present invention is (x) a compound of formula (I) according to (i), or a pharmaceutically acceptable salt thereof, wherein A₄ is CH.

A further embodiment of the present invention is (xi) a compound of formula (I) according to (i), or a pharmaceutically acceptable salt thereof, wherein X₂ is

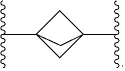 [SMILES: *C12CC(*)(C1)C2]

A further embodiment of the present invention is (xii) a compound of formula (II) according to (i), or a pharmaceutically acceptable salt thereof,

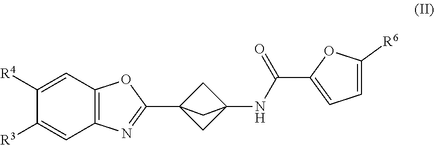

wherein
R³ is selected from H and halogen;
R⁴ is selected from H and halogen;
R⁶ is selected from C₃₋₇cycloalkylsulfonyl, C₃₋₇cycloalkylC₁₋₆alkylsulfonyl, C₃₋₇cycloalkylsulfonimidoyl, C₁₋₆alkylsulfonimidoyl, C₃₋₇cycloalkylC₁₋₆alkylsulfonimidoyl and C₁₋₆alkylsulfonylC₁₋₆alkyl.

A further embodiment of the present invention is (xiii) a compound of formula (II) according to (i), or a pharmaceutically acceptable salt thereof, wherein
R³ is selected from H and Cl;
R⁴ is selected from H and Cl;
R⁶ is selected from cyclopropylsulfonyl, cyclopropylmethylsulfonyl, cyclopropylsulfonimidoyl, methylsulfonimidoyl, cyclopropylmethylsulfonimidoyl and methylsulfonylmethyl.

A further embodiment of the present invention is (xiv) a compound of formula (II) according to (i), or a pharmaceutically acceptable salt thereof, wherein
R³ is selected from H;
R⁴ is selected from Cl;
R⁶ is selected from cyclopropylsulfonyl, cyclopropylmethylsulfonyl, cyclopropylsulfonimidoyl, methylsulfonimidoyl, cyclopropylmethylsulfonimidoyl and methylsulfonylmethyl.

A further embodiment of the present invention is (xv) a compound of formula (II) according to (i), or a pharmaceutically acceptable salt thereof, wherein
R³ is selected from Cl;
R⁴ is selected from H;
R⁶ is selected from cyclopropylsulfonyl, cyclopropylmethylsulfonyl, cyclopropylsulfonimidoyl, methylsulfonimidoyl, cyclopropylmethylsulfonimidoyl and methylsulfonylmethyl.

In another embodiment (xvi) of the present invention, particular compounds of the present invention are selected from:
N-[3-(5-chloro-1,3-benzoxazol-2-yl)-1-bicyclo[1.1.1]pentanyl]-5-methylsulfonyl-furan-2-carboxamide;
N-[3-(5-chloro-1,3-benzoxazol-2-yl)-1-bicyclo[1.1.1]pentanyl]-5-methylsulfinyl-furan-2-carboxamide;
N-[3-(5-chloro-1,3-benzoxazol-2-yl)-1-bicyclo[1.1.1]pentanyl]-5-[(S)-methylsulfinyl]furan-2-carboxamide;
N-[3-(5-chloro-1,3-benzoxazol-2-yl)-1-bicyclo[1.1.1]pentanyl]-5-[(R)-methylsulfinyl]furan-2-carboxamide;
N-[3-(5-chloro-1,3-benzoxazol-2-yl)-1-bicyclo[1.1.1]pentanyl]-5-methylsulfanyl-furan-2-carboxamide;
N-[3-(5-chloro-1,3-benzoxazol-2-yl)-1-bicyclo[1.1.1]pentanyl]-5-cyclopropylsulfinyl-furan-2-carboxamide;
N-[3-(5-chloro-1,3-benzoxazol-2-yl)-1-bicyclo[1.1.1]pentanyl]-5-cyclopropylsulfonyl-furan-2-carboxamide;
N-[3-(5-chloro-1,3-benzoxazol-2-yl)-1-bicyclo[1.1.1]pentanyl]-5-(cyclopropylmethylsulfinyl)furan-2-carboxamide;
N-[3-(5-chloro-1,3-benzoxazol-2-yl)-1-bicyclo[1.1.1]pentanyl]-5-(cyclopropylmethylsulfonyl)furan-2-carboxamide;
5-bromo-N-[3-(5-chloro-1,3-benzoxazol-2-yl)-1-bicyclo[1.1.1]pentanyl]furan-2-carboxamide;
N-[3-(5-chloro-1,3-benzoxazol-2-yl)-1-bicyclo[1.1.1]pentanyl]-5-methylsulfonyl-thiophene-2-carboxamide;
N-[3-(5-chloro-1,3-benzoxazol-2-yl)-1-bicyclo[1.1.1]pentanyl]-5-sulfamoyl-furan-2-carboxamide;
N-[3-(5-chloro-1,3-benzoxazol-2-yl)-1-bicyclo[1.1.1]pentanyl]-5-(cyclopropylsulfonimidoyl)furan-2-carboxamide;
N-[3-(5-chloro-1,3-benzoxazol-2-yl)-1-bicyclo[1.1.1]pentanyl]-5-(methylsulfonimidoyl)furan-2-carboxamide;
N-[3-(5-chloro-1,3-benzoxazol-2-yl)-1-bicyclo[1.1.1]pentanyl]-5-[(R)-methylsulfonimidoyl]furan-2-carboxamide;
N-[3-(5-chloro-1,3-benzoxazol-2-yl)-1-bicyclo[1.1.1]pentanyl]-5-[(S)-methylsulfonimidoyl]furan-2-carboxamide;
N-[3-(5-chloro-1,3-benzoxazol-2-yl)-1-bicyclo[1.1.1]pentanyl]-5-(cyclopropylmethylsulfonimidoyl)furan-2-carboxamide;
N-[3-(5-chloro-1,3-benzoxazol-2-yl)-1-bicyclo[1.1.1]pentanyl]-5-(oxetan-3-ylmethylsulfonyl)furan-2-carboxamide;
N-[3-(5-chloro-1,3-benzoxazol-2-yl)-1-bicyclo[1.1.1]pentanyl]-5-(oxetan-3-ylmethylsulfonimidoyl)furan-2-carboxamide;
N-[3-(5-chloro-1,3-benzoxazol-2-yl)-1-bicyclo[1.1.1]pentanyl]-5-(methylsulfonylmethyl)furan-2-carboxamide;
N-[3-(5-chloro-1,3-benzoxazol-2-yl)-1-bicyclo[1.1.1]pentanyl]-5-[(methylsulfonimidoyl)methyl]furan-2-carboxamide;
N-[3-(5-chloro-1,3-benzoxazol-2-yl)-1-bicyclo[1.1.1]pentanyl]-5-(sulfamoylmethyl)furan-2-carboxamide;
3-bromo-N-[3-(5-chloro-1,3-benzoxazol-2-yl)-1-bicyclo[1.1.1]pentanyl]benzamide;
4-bromo-N-[3-(5-chloro-1,3-benzoxazol-2-yl)-1-bicyclo[1.1.1]pentanyl]benzamide;
N-[3-(5-chloro-1,3-benzoxazol-2-yl)-1-bicyclo[1.1.1]pentanyl]-3-methylsulfanyl-benzamide;
N-[3-(5-chloro-1,3-benzoxazol-2-yl)-1-bicyclo[1.1.1]pentanyl]-3-methylsulfonyl-benzamide;
N-[3-(5-chloro-1,3-benzoxazol-2-yl)-1-bicyclo[1.1.1]pentanyl]-2-(trifluoromethyl)pyridine-4-carboxamide;
N-[3-(5-chloro-1,3-benzoxazol-2-yl)-1-bicyclo[1.1.1]pentanyl]-5-(trifluoromethyl)pyridine-3-carboxamide;
N-[3-(5-chloro-1,3-benzoxazol-2-yl)-1-bicyclo[1.1.1]pentanyl]-6-(trifluoromethyl)pyridine-3-carboxamide;
N-[3-(5-chloro-1,3-benzoxazol-2-yl)-1-bicyclo[1.1.1]pentanyl]-2-(trifluoromethyl)pyrimidine-5-carboxamide;
N-[3-(5-chloro-1,3-benzoxazol-2-yl)-1-bicyclo[1.1.1]pentanyl]-6-(trifluoromethyl)pyridazine-3-carboxamide;
2,5-dichloro-N-[3-(5-chloro-1,3-benzoxazol-2-yl)-1-bicyclo[1.1.1]pentanyl]benzamide;
N-[3-(5-chloro-1,3-benzoxazol-2-yl)-1-bicyclo[1.1.1]pentanyl]-3-(methylsulfonimidoyl)benzamide;
N-[3-(5-chloro-1,3-benzoxazol-2-yl)-1-bicyclo[1.1.1]pentanyl]-2,2-dioxo-2lambda6-thiaspiro[3.3]heptane-6-carboxamide;
5-(butanoylsulfamoyl)-N-[3-(5-chloro-1,3-benzoxazol-2-yl)-1-bicyclo[1.1.1]pentanyl]furan-2-carboxamide;
N-[3-(6-chloro-1,3-benzoxazol-2-yl)-1-bicyclo[1.1.1]pentanyl]-5-methylsulfonyl-furan-2-carboxamide;
N-[3-(6-chloro-1,3-benzoxazol-2-yl)-1-bicyclo[1.1.1]pentanyl]-5-cyclopropylsulfonyl-furan-2-carboxamide;
N-[3-(6-chloro-1,3-benzoxazol-2-yl)-1-bicyclo[1.1.1]pentanyl]-5-(cyclopropylmethylsulfonyl)furan-2-carboxamide;
N-[3-(6-chloro-1,3-benzoxazol-2-yl)-1-bicyclo[1.1.1]pentanyl]-5-(methylsulfonimidoyl)furan-2-carboxamide;
N-[3-(6-chloro-1,3-benzoxazol-2-yl)-1-bicyclo[1.1.1]pentanyl]-3-methylsulfonyl-benzamide;
N-[3-(6-chloro-1,3-benzoxazol-2-yl)-1-bicyclo[1.1.1]pentanyl]-2-(trifluoromethyl)pyridine-4-carboxamide;
N-[3-(6-chloro-1,3-benzoxazol-2-yl)-1-bicyclo[1.1.1]pentanyl]-2-cyclopropylsulfonyl-pyridine-4-carboxamide;
N-[3-(6-chloro-1,3-benzoxazol-2-yl)-1-bicyclo[1.1.1]pentanyl]-5-(methylsulfonylmethyl)furan-2-carboxamide;
N-[3-(6-chloro-1,3-benzoxazol-2-yl)-1-bicyclo[1.1.1]pentanyl]-5-[(methylsulfonimidoyl)methyl]furan-2-carboxamide;
N-[3-(6-chlorooxazolo[5,4-b]pyridin-2-yl)-1-bicyclo[1.1.1]pentanyl]-5-(methylsulfonylmethyl)furan-2-carboxamide;
N-[3-(6-chlorooxazolo[5,4-b]pyridin-2-yl)-1-bicyclo[1.1.1]pentanyl]-2-(trifluoromethyl)pyridine-4-carboxamide;
N-[3-(6-chlorooxazolo[4,5-c]pyridin-2-yl)-1-bicyclo[1.1.1]pentanyl]-5-(methylsulfonylmethyl)furan-2-carboxamide;
N-[3-(7-chloro-1,3-benzoxazol-2-yl)-1-bicyclo[1.1.1]pentanyl]-5-methylsulfonyl-furan-2-carboxamide;
N-[3-(6-fluoro-1,3-benzoxazol-2-yl)-1-bicyclo[1.1.1]pentanyl]-5-methylsulfonyl-furan-2-carboxamide;
N-[4-(5-chloro-1,3-benzoxazol-2-yl)norboman-1-yl]-5-methylsulfonyl-furan-2-carboxamide;
N-[4-(5-chloro-1,3-benzoxazol-2-yl)-1-bicyclo[2.2.2]octanyl]-5-(trifluoromethyl)furan-2-carboxamide;
N-[4-(5-chloro-1,3-benzoxazol-2-yl)-1-bicyclo[2.2.2]octanyl]-2,2-dioxo-2lambda6-thiaspiro[3.3]heptane-6-carboxamide;
N-[4-(5-chloro-1,3-benzoxazol-2-yl)-1-bicyclo[2.2.2]octanyl]-1,1-dioxo-thiolane-2-carboxamide;
N-[4-(5-chloro-1,3-benzoxazol-2-yl)-1-bicyclo[2.2.2]octanyl]-1,1-dioxo-thiolane-3-carboxamide;
N-[4-(5-chloro-1,3-benzoxazol-2-yl)-1-bicyclo[2.2.2]octanyl]-3-methyl-1,1-dioxo-thiolane-3-carboxamide;
N-[4-(5-chloro-1,3-benzoxazol-2-yl)-1-bicyclo[2.2.2]octanyl]-1,1-dioxo-thiane-3-carboxamide;
N-[4-(5-chloro-1,3-benzoxazol-2-yl)-1-bicyclo[2.2.2]octanyl]-2-(1,1-dioxothiolan-2-yl)acetamide;
N-[4-(5-chloro-1,3-benzoxazol-2-yl)-1-bicyclo[2.2.2]octanyl]-2-(1,1-dioxothian-3-yl)acetamide; and
N-[4-(5-chloro-1,3-benzoxazol-2-yl)-1-bicyclo[2.2.2]octanyl]-2-(1,1-dioxothiazinan-2-yl)acetamide;
or a pharmaceutically acceptable salt thereof.

In another embodiment (xvii) of the present invention, particular compounds of the present invention are selected from:
N-[3-(5-chloro-1,3-benzoxazol-2-yl)-1-bicyclo[1.1.1]pentanyl]-5-cyclopropylsulfonyl-furan-2-carboxamide;
N-[3-(5-chloro-1,3-benzoxazol-2-yl)-1-bicyclo[1.1.1]pentanyl]-5-(cyclopropylmethylsulfonyl)furan-2-carboxamide;
N-[3-(5-chloro-1,3-benzoxazol-2-yl)-1-bicyclo[1.1.1]pentanyl]-5-(cyclopropylsulfonimidoyl)furan-2-carboxamide;
N-[3-(5-chloro-1,3-benzoxazol-2-yl)-1-bicyclo[1.1.1]pentanyl]-5-(methylsulfonimidoyl)furan-2-carboxamide;
N-[3-(5-chloro-1,3-benzoxazol-2-yl)-1-bicyclo[1.1.1]pentanyl]-5-(cyclopropylmethylsulfonimidoyl)furan-2-carboxamide;
N-[3-(5-chloro-1,3-benzoxazol-2-yl)-1-bicyclo[1.1.1]pentanyl]-5-(methylsulfonylmethyl)furan-2-carboxamide;
N-[3-(6-chloro-1,3-benzoxazol-2-yl)-1-bicyclo[1.1.1]pentanyl]-5-cyclopropylsulfonyl-furan-2-carboxamide; and
N-[3-(6-chloro-1,3-benzoxazol-2-yl)-1-bicyclo[1.1.1]pentanyl]-5-(methylsulfonylmethyl)furan-2-carboxamide;
or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, A₁ to A₄, X₁, X₂ and R¹ are as defined above unless otherwise indicated. Furthermore, and unless explicitly otherwise stated, all reactions, reaction conditions, abbreviations and symbols have the meanings well known to a person of ordinary skill in organic chemistry.

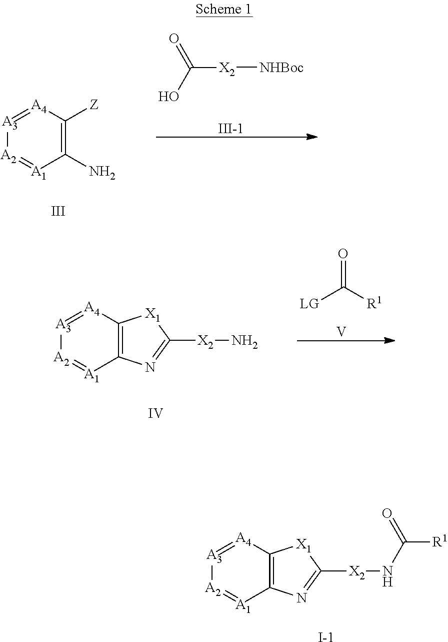

Wherein Z is halogen or OH, LG is OH or halogen.

Compound of formula III is heated with a carboxylic acid III-1 in the presence of an acid, such as polyphosphoric acid, to give compound of formula IV, which then reacts with compound of formula V in the presence of a coupling reagent, such as EDCI, HATU or T₃P, and a base, such as DMAP, TEA or DIPEA, in a solvent, such as DMF or DCM, to afford compound of formula I-1.

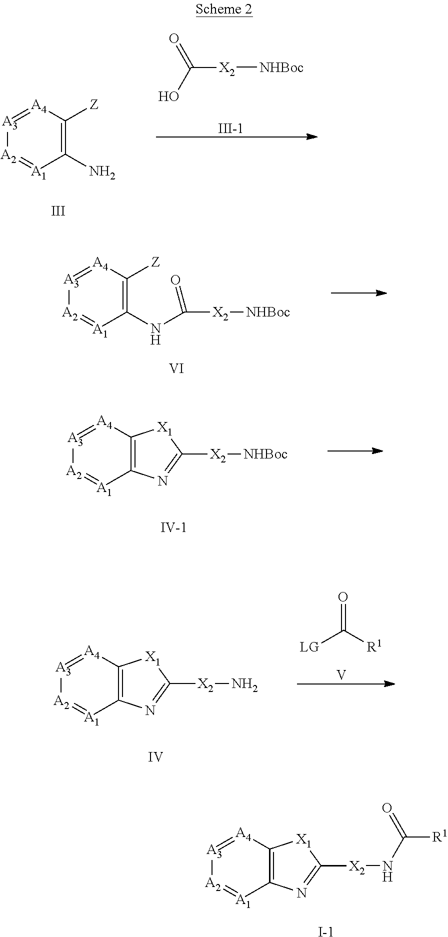

Wherein Z is halogen or OH, LG is OH or halogen.

Compound of formula III reacts with a carboxylic acid III-1 in the presence of a coupling reagent, such as EDCI, and a base, such as DMAP, in a solvent, such as DCM, to afford compound of formula VI. Cyclization of compound of formula VI with DEAD and PPh₃, in a suitable solvent, such as THF, to afford compound of formula IV-1, which is then deprotected with a suitable acid, such as TFA, to afford compound of formula IV. Coupling of compound of formula IV with compound of formula V in the presence of a coupling reagent, such as EDCI, HATU or T₃P, and a base, such as DMAP, TEA or DIPEA, in a solvent, such as DMF or DCM, to afford compound of formula I-1.

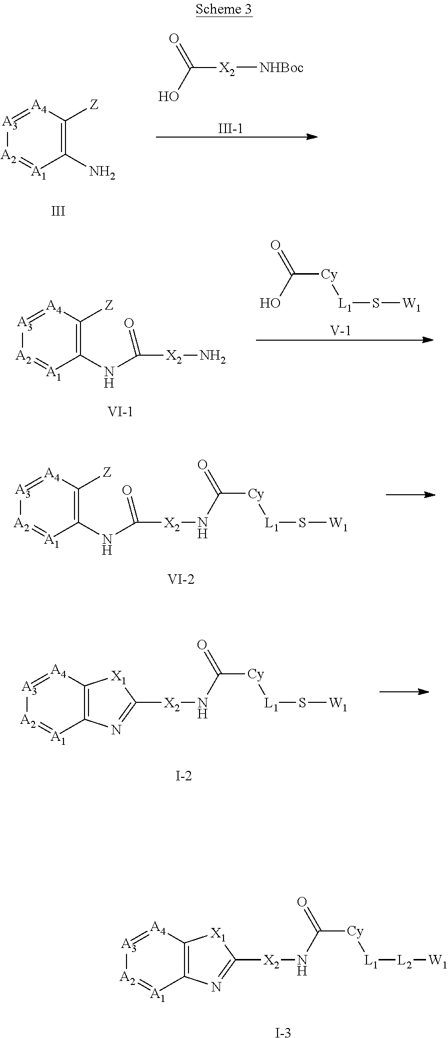

Wherein Z is halogen or OH; Cy is furyl or phenyl; L₁ is —CH₂— or a bond; L₂ is S(O) or S(O)₂; W₁ is C₁₋₆alkyl, C₃₋₇cycloalkyl, C₃₋₇cycloalkylC₁₋₆alkyl, heterocyclyl or heterocyclylC₁₋₆alkyl.

Compound of formula III is heated with a carboxylic acid III-1 in the presence of an acid, such as polyphosphoric acid, to give compound of formula VI-1, which then reacts with compound of formula V-1 in the presence of a coupling reagent, such as HATU or T₃P, and a base, such as TEA or DIPEA, in a solvent, such as DMF or DCM, to afford compound of formula VI-2. Cyclization of compound of formula VI-2 with microwave irradiation in the presence of a base such as K₂CO₃, in a suitable solvent, such as NMP, affords compound of formula I-2. Oxidation of compound of formula I-2 in the presence of an oxidate, such as m-CPBA, in a suitable solvent, such as DCM, affords compound of formula I-3.

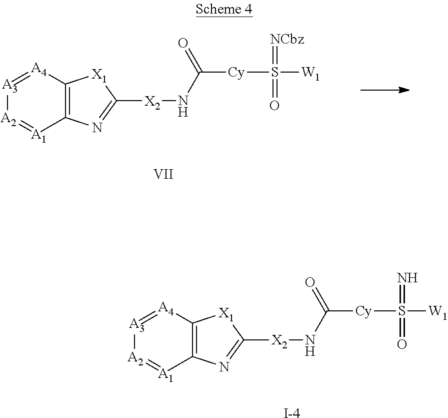

Wherein Cy is furyl or phenyl; W₁ is C₁₋₆alkyl, C₃₋₇cycloalkyl, C₃₋₇cycloalkylC₁₋₆alkyl, heterocyclyl or heterocyclylC₁₋₆alkyl.

Deprotection of compound of formula VII with TMSI, in the presence of a base, such as DIPEA, in a suitable solvent, such as MeCN, affords compound of formula I-4.

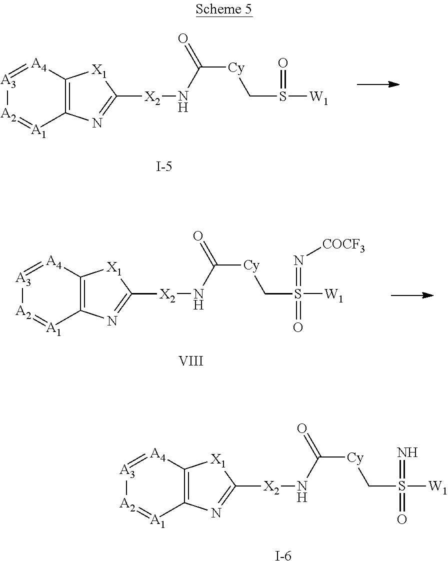

Wherein Cy is furyl or phenyl; W₁ is C₁₋₆alkyl, C₃₋₇cycloalkyl, C₃₋₇cycloalkylC₁₋₆alkyl, heterocyclyl or heterocyclylC₁₋₆alkyl.

Compound of formula I-5 reacts with 2,2,2-trifluoroacetamide in the presence of magnesium oxide, rhodium(II) acetate dimer and iodobenzene diacetate, in a suitable solvent, such as DCM, affords compound of formula VIII, which is then deprotected with a base, such as K₂CO₃, in a suitable solvent such as MeOH, to afford compound of formula I-6.

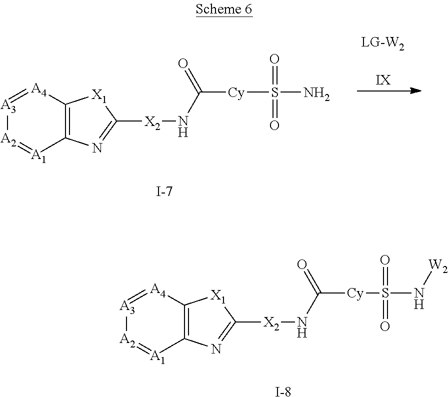

Wherein LG is halogen; Cy is furyl or phenyl; W₂ is C₁₋₆alkylcarbonyl.

Compound of formula I-7 reacts with a halide VIII in the presence of a base, such as K₂CO₃, DMAP or TEA, in a solvent, such as DCM, to afford compound of formula I-8.

This invention also relates to a process for the preparation of a compound of formula (I) comprising at least one of the following steps:
(a) Coupling of a compound of formula (IV),

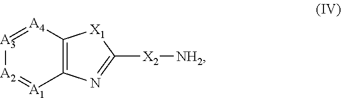

with a compound of formula (V),

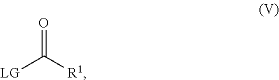

in the presence of a coupling reagent and a base;
(b) Cyclization of a compound of formula (VI-2),

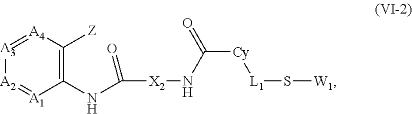

in the presence of a base;
(c) Oxidation of a compound of formula (I-2),

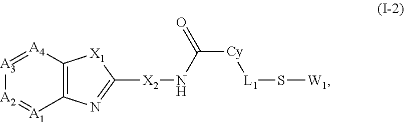

(d) Deprotection of a compound of formula (VII),

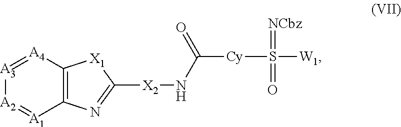

with TMSI, in the presence of a base;
(e) Deprotection of a compound of formula (VIII),

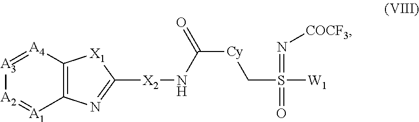

in the presence of a base;
(f) Reaction of a compound of formula (I-7),

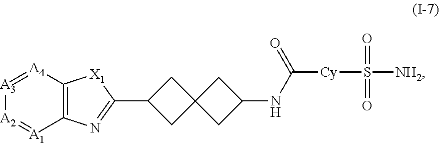

with a halide (IX), LG-W₂ (IX), in the presence of a base;
wherein A₁ to A₄, X₁, X₂ and R¹ are defined above; Z is halogen or OH; Cy is furyl or phenyl; LG is OH or halogen; L₁ is —CH₂— or a bond; W₁ is C₁₋₆alkyl, C₃₋₇cycloalkyl, C₃₋₇cycloalkylC₁₋₆ alkyl, heterocyclyl or heterocyclylC₁₋₆alkyl; W₂ is C₁₋₆alkylcarbonyl.
The coupling reagent in step (a) can be for example EDCI, HATU or T₃P;
The base in step (a) can be for example DMAP, TEA or DIPEA;
The base in step (b) can be for example K₂CO₃;
The oxidate in step (c) can be for example m-CPBA;
The base in step (d) can be for example DIPEA;
The base in step (e) can be for example K₂CO₃;
The base in step (f) can be for example K₂CO₃, DMAP or TEA.

A compound of formula (I) or (II) when manufactured according to the above process is also an object of the invention.

The compound of this invention also shows good safety and PK profile.

### Pharmaceutical Compositions and Administration

The invention also relates to a compound of formula (I) or (II) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I) or (II), or a pharmaceutically acceptable salt thereof.

In a further embodiment includes a pharmaceutical composition comprising a compound of formula (I) or (II), or a pharmaceutically acceptable salt thereof, together with a pharmaceutically acceptable carrier or excipient.

Another embodiment includes a pharmaceutical composition comprising a compound of formula (I) or (II), or a pharmaceutically acceptable salt thereof for use in the treatment of HBV infection.

### Indications and Methods of Treatment

The compounds of the invention have anti-HBV activity. Accordingly, the compounds of the invention are useful for the treatment or prophylaxis of HBV infection.

The invention also relates to the use of a compound of formula (I) or (II) for the inhibition of HBeAg.

The invention further relates to the use of a compound of formula (I) or (II) for the inhibition of HBsAg.

The invention relates to the use of a compound of formula (I) or (II) for the inhibition of HBV DNA.

The invention relates to the use of a compound of formula (I) or (II) for use in the treatment or prophylaxis of HBV infection.

The use of a compound of formula (I) or (II) for the preparation of medicaments useful in the treatment or prophylaxis diseases that are related to HBV infection is an object of the invention.

The invention relates in particular to the use of a compound of formula (I) or (II) for the preparation of a medicament for the treatment or prophylaxis of HBV infection.

Another embodiment includes a method for the treatment or prophylaxis of HBV infection, which method comprises administering an effective amount of a compound of formula (I) or (II), or a pharmaceutically acceptable salt thereof.

The invention relates in particular to a compound of formula (I) and (II) for use in the treatment or prophylaxis of HBV infection.

### EXAMPLES

The invention will be more fully understood by reference to the following examples. They should not, however, be construed as limiting the scope of the invention.

Abbreviations used herein are as follows:
ACN: acetonitrile
CDCl₃: deuterated chloroform
CD₃OD: deuterated methanol
DEAD: diethyl azodicarboxylate
DIPEA: N,N-diisopropylethylamine
DMAP 4-dimethylaminopyridine
DMF: dimethylformamide
DMSO-d₆: deuterated dimethylsulfoxide
EDCI: 1-(3-dimethylaminopropyl)-3-ethylcarbodiimide hydrochloride
EtOAc: ethyl acetate
HATU O-(7-aza-1H-benzotriazole-1-yl)-N,N,N′,N′-tetramethyluronium hexafluorophosphate
HPLC: high performance liquid chromatography
h: hour
IC₅₀: the half maximal inhibitory concentration
LC/MS: Liquid chromatography/mass spectrometry
MeOH: methanol
M: molarity
m-CPBA: 3-chloroperoxybenzoic acid
MHz: megahertz
min: minute
mL: milliliter
mmol: millimole
MS (ESI): mass spectroscopy (electron spray ionization)
NMP: N-methyl pyrrolidone
NMR: nuclear magnetic resonance
obsd. Observed
PPh₃: triphenylphosphine
SFC: supercritical fluid chromatography
TEA: triethylamine
TFA: trifluoroacetic acid
THF: tetrahydrofuran
TLC: thin layer chromatography
TMSI: iodotrimethylsilane
T₃P: 2,4,6-tripropyl-1,3,5,2,4,6-trioxatriphosphinane 2,4,6-trioxide
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using an Acquity Ultra Performance LC—3100 Mass Detector or Acquity Ultra Performance LC—SQ Detector. Standard LC/MS conditions were as follows (running time 3 minutes):
Acidic condition: A: 0.1% formic acid in H₂O; B: 0.1% formic acid in acetonitrile;
Basic condition: A: 0.05% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)⁺.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

The invention will be more fully understood by reference to the following examples. They should not, however, be construed as limiting the scope of the invention.

### Intermediate Int-1

### 5-methylsulfanylfuran-2-carboxylic acid

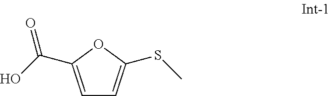

The title compound was prepared according to the following scheme:

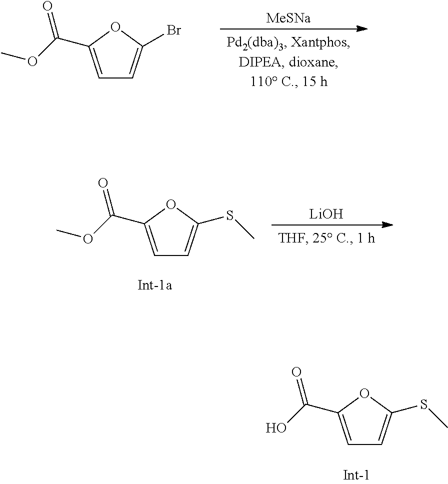

Step 1: Preparation of Methyl 5-methylsulfanylfuran-2-carboxylate (Int-1a)

A mixture of methyl 5-bromo-2-furoate (10 g, 48.78 mmol), sodium thiomethoxide (6.84 g, 97.56 mmol), 9,9-dimethyl-4,5-bis(diphenylphosphino)xanthene (2.82 g, 4.88 mmol), N,N-diisopropylethylamine (25.49 mL, 146.33 mmol) and tris(dibenzylideneacetone)dipalladium (0) (2.23 g, 2.44 mmol) in 1,4-dioxane (200 mL) was stirred at 110° C. for 15 h. The solvent was evaporated in vacuo and the EtOAc (300 mL) was added. The resulting mixture was filtered by a short silica gel column. The filtrate was concentrated and purified by flash column (eluting with EtOAc/PE=3/97) to afford methyl 5-methylsulfanylfuran-2-carboxylate as a yellow oil (Int-1a, 7 g, 83.3%). MS obsd. (ESI⁺) [(M+H)⁺]: 173.1.

Step 2: Preparation of Methyl 5-methylsulfinylfuran-2-carboxylic acid (Int-1)

To a solution of methyl 5-methylsulfanylfuran-2-carboxylate (Int-1a, 7 g, 40.65 mmol) in THF (10 mL) was added LiOH (2.5 M, 19.5 mL). After being stirred at 25° C. for 1 h, the reaction mixture was concentrated under reduced pressure to remove the organic solvent. The aqueous layer was extracted with EtOAc (100 mL×2), and then acidified with aqueous HCl until pH=3 to 4. The precipitate was collected by filtration and dried in vacuo to afford 5-methylsulfanylfuran-2-carboxylic acid as a light yellow solid (Int-1, 5.6 g, 87.1%). MS obsd. (ESI⁺) [(M+H)⁺]: 159.1. ¹H NMR (400 MHz, CDCl₃) δ ppm: 7.30 (d, J=3.6 Hz, 1H), 6.40 (d, J=3.6 Hz, 1H), 2.55 (s, 3H).

### Intermediate Int-2

### 5-methylsulfinylfuran-2-carboxylic acid

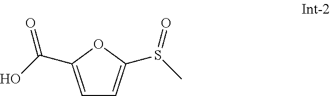

The title compound was prepared according to the following scheme:

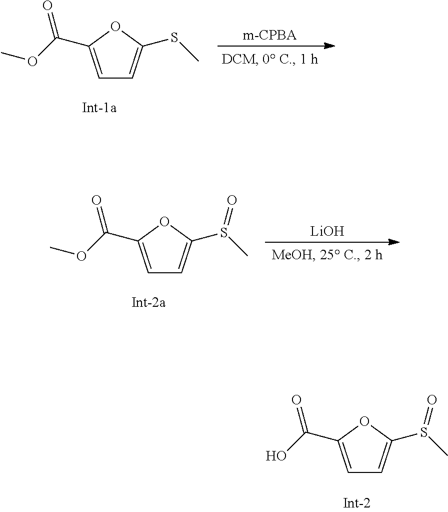

### Preparation of Methyl 5-methylsulfinylfuran-2-carboxylate (Int-2a)

To a solution of methyl 5-methylsulfanylfuran-2-carboxylate (Int-1a, 4.5 g, 26.1 mmol) in DCM (10 mL) was added 3-chloroperoxybenzoic acid (4.5 g, 26.1 mmol). After being stirred at 0° C. for 1 h, the mixture was washed with saturated Na₂CO₃. The organic layer was dried over Na₂SO₄, filtered and concentrated in vacuo. The residue was purified by flash column (eluting with EtOAc/PE=1/2) to afford methyl 5-methylsulfinylfuran-2-carboxylate as a light yellow solid (Int-2a, 3.7 g, 75%). MS obsd. (ESI⁺) [(M+H)⁺]: 189.1.

Step 2: Preparation of 5-methylsulfinylfuran-2-carboxylic acid (Int-2)

To a solution of methyl 5-methylsulfinylfuran-2-carboxylate (Int-2a, 90 mg, 0.48 mmol) in a mixed solvent of MeOH (10 mL) and water (10 mL) was added LiOH·H₂O (134 mg, 2.4 mmol). After being stirred at 25° C. for 2 h, the MeOH was evaporated. The residue was acidified by HCl (1 M) to pH=2 and extracted with DCM (15 mL×3). The combined organic layer was dried over Na₂SO₄, filtered and concentrated in vacuo to afford 5-methylsulfinylfuran-2-carboxylic acid as a yellow oil (Int-2, 60 mg, 72%). MS obsd. (ESI⁺) [(M+H)⁺]: 175.1.

### Intermediate Int-3

### 5-methylsulfonylfuran-2-carboxylic acid

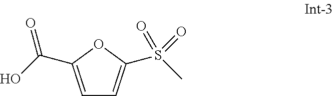

The title compound was prepared according to the following scheme:

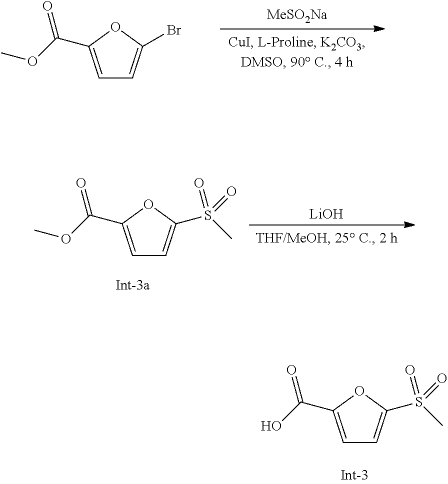

Step 1: Preparation of Methyl 5-methylsulfonylfuran-2-carboxylate (Int-3a)

To a solution of methyl 5-bromo-2-furoate (2.05 g, 10 mmol) in DMSO (25 mL) was added methylsulfinyloxysodium (1.23 g, 12 mmol) followed by copper (I) iodide (380.9 mg, 2 mmol), L-proline (460.5 mg, 4 mmol) and K₂CO₃ (414.6 mg, 3 mmol). The resulting mixture was heated with stirring at 90° C. for 4 h. The reaction was diluted with water (30 mL) and extracted with EtOAc (30 mL×3). The combined organic layer was washed with brine (50 mL×2), dried over Na₂SO₄, filtered and concentrated in vacuo. The residue was purified by flash column (eluting with EtOAc/PE=1/3) to afford methyl 5-methylsulfonylfuran-2-carboxylate as a white solid (Int-3a, 750 mg, 36.7%). MS obsd. (ESI⁺) [(M+H)⁺]: 205.1.

Step 2: Preparation of 5-methylsulfonylfuran-2-carboxylic acid (Int-3)

To a solution of methyl 5-methylsulfonylfuran-2-carboxylate (Int-3a, 750 mg, 3.7 mmol) in a mixed solvent of THF (25 mL) and MeOH (25 mL) was added an aqueous of LiOH·H₂O (2 M, 55 mL). After being stirred at 25° C. for 2 h, most of the solvent was evaporated. The residue was acidified by HCl (2 M) to pH=3 and extracted with EtOAc (20 mL×3). The combined organic layer was dried over Na₂SO₄, filtered and concentrated in vacuo to afford 5-methylsulfonylfuran-2-carboxylic acid as a white solid (Int-3, 655 mg, 89.1%). MS obsd. (ESI⁺) [(M+H)⁺]: 191.1.

### Intermediate Int-4

### 5-cyclopropylsulfanylfuran-2-carboxylic acid

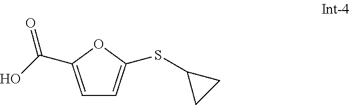 [SMILES: O=C(O)c1ccc(SC2CC2)o1]

The title compound was prepared according to the following scheme:

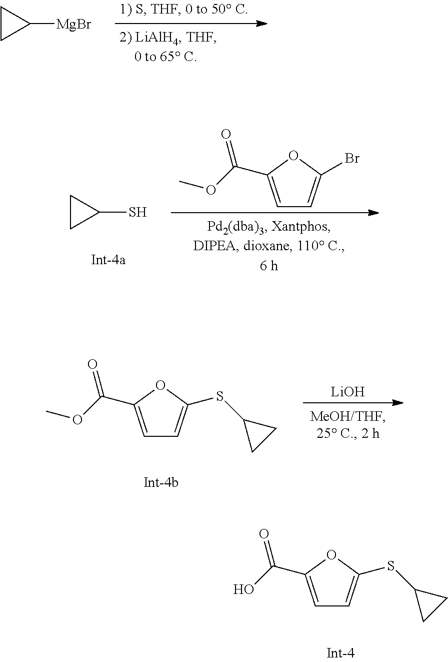

### Step 1: Preparation of Cyclopropanethiol (Int-4a)

To a solution of cyclopropylmagnesium bromide (10 mL, 5 mmol) in THF was added sulfur (160 mg, 0.63 mmol) at 0° C. Then the solution was heated at 50° C. with stirring for 3 h. After being cooled in an ice-bath, Lithium aluminum hydride (5 mL, 5 mmol) in THF was added. The resulting mixture was stirred at 65° C. for 0.5 h and quenched by H₂O (0.5 mL) at 0° C., then acidified by H₂SO₄ (5% v/v, 20 mL). The organic phase containing cyclopropanethiol (Int-4a) was separated, dried over anhydrous Na₂SO₄ and used for the next step without purification.

Step 2: Preparation of Methyl 5-cyclopropylsulfanylfuran-2-carboxylate (Int-4b)

To a mixture of methyl 5-bromo-2-furoate (410 mg, 2 mmol), tris(dibenzylideneacetone)dipalladium (0) (183 mg, 0.2 mmol), 9,9-dimethyl-4,5-bis(diphenylphosphino)xanthene (116 mg, 0.2 mmol) and N,N-diisopropylethylamine (1.74 mL, 10 mmol) in 1,4-dioxane (15 mL) was added the above solution containing cyclopropanethiol (Int-5a). After being stirred at 110° C. for 6 h, the mixture was filtered with a short silica gel colunm. The filtration was concentrated and purified by flash column (eluting with EtOAc/PE=1/99) to afford methyl 5-cyclopropylsulfanylfuran-2-carboxylate as a colorless oil (Int-4b, 120 mg, 12%). MS obsd. (ESI⁺) [(M+H)⁺]: 199.1.

Step 3: Preparation of 5-cyclopropylsulfanylfuran-2-carboxylic acid (Int-4)

To a solution of methyl 5-cyclopropylsulfanylfuran-2-carboxylate (Int-4b, 50 mg, 0.25 mmol) in a mixed solvent of THF (2 mL) and MeOH (2 mL) was added a solution of LiOH in water (1.9 mL, 2 M). After being stirred at 25° C. for 2 h, the mixture was acidified by HCl (1 M) to pH=5, then extracted by EtOAc (10 mL×3). The combined organic layer was dried over Na₂SO₄, filtered and concentrated to afford 5-cyclopropylsulfanylfuran-2-carboxylic acid as a white solid (Int-4, 46 mg, 99%). MS obsd. (ESI⁺) [(M+H)⁺]: 185.1.

### Intermediate Int-5

### 5-cyclopropylsulfinylfuran-2-carboxylic acid

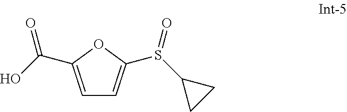 [SMILES: O=C(O)c1ccc(S(=O)C2CC2)o1]

The title compound was prepared in analogy to the procedure described for the preparation of 5-methylsulfinylfuran-2-carboxylic acid (Int-2), by using methyl 5-cyclopropylsulfanylfuran-2-carboxylate instead of methyl 5-methylsulfanylfuran-2-carboxylate. MS obsd. (ESI⁺) [(M+H)⁺]: 201.1.

### Intermediate Int-6

### 5-cyclopropylsulfonylfuran-2-carboxylic acid

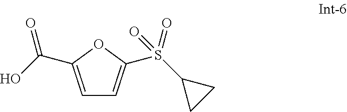 [SMILES: O=C(O)c1ccc(S(=O)(=O)C2CC2)o1]

The title compound was prepared in analogy to the procedure described for the preparation of 5-methylsulfonylfuran-2-carboxylic acid (Int-3), by using cyclopropylsulfinyloxysodium instead of methylsulfinyloxysodium. MS obsd. (ESI⁺) [(M+H)⁺]: 217.1.

### Intermediate Int-7

### 5-(cyclopropylmethylsulfanyl)furan-2-carboxylic acid

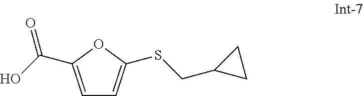 [SMILES: O=C(O)c1ccc(SCC2CC2)o1]

The title compound was prepared according to the following scheme:

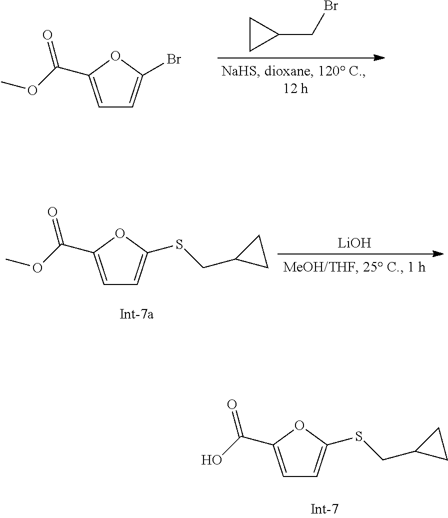

Step 1: Preparation of Methyl 5-(cyclopropylmethylsulfanyl)furan-2-carboxylate (Int-7a)

To a solution of methyl 5-bromo-2-furoate (2 g, 9.76 mmol) in 1,4-dioxane (20 mL) was added sodium hydrosulfide (5.5 g, 97.6 mmol) and (bromomethyl)cyclopropane (3.32 mL, 34.1 mmol). After being stirred at 120° C. for 12 h, the reaction mixture was quenched with H₂O (50 mL) and extracted with DCM (50 mL×3). The combined organic layer was washed with brine, dried over Na₂SO₄ and concentrated in vacuo. The residue was purified by flash column (eluting with 100% PE) to afford methyl 5-(cyclopropylmethylsulfanyl)furan-2-carboxylate as a colorless oil (Int-7a, 580 mg, 28%). MS obsd. (ESI⁺) [(M+H)⁺]: 213.1. ¹H NMR (400 MHz, CDCl₃) δ ppm: 7.14 (d, J=3.5 Hz, 1H), 6.50 (d, J=3.5 Hz, 1H), 3.89 (s, 3H), 2.86 (d, J=7.2 Hz, 2H), 0.95-1.10 (m, 1H), 0.51-0.61 (m, 2H), 0.14-0.24 (m, 2H).

Step 2: Preparation of 5-(cyclopropylmethylsulfanyl)furan-2-carboxylic acid (Int-7)

To a solution of methyl 5-(cyclopropylmethylsulfanyl)furan-2-carboxylate (Int-7a, 308 mg, 1.45 mmol) in a mixed solvent of THF (2 mL) and methanol (2 mL) was added a solution of LiOH in water (2.2 mL, 2 M). After being stirred at 20° C. for 1 h, the mixture was acidified by HCl (2.5 mL, 2 M). The solvent was evaporated and the residue was separated by EtOAc (20 mL) and water (20 mL). The organic layer was dried over Na₂SO₄, filtered and concentrated to afford the 5-(cyclopropylmethylsulfanyl)furan-2-carboxylic acid as a white solid (Int-7, 280 mg, 97%). MS obsd. (ESI⁺) [(M+H)⁺]: 199.1.

### Intermediate Int-8

### 5-(cyclopropylmethylsulfinyl)furan-2-carboxylic acid

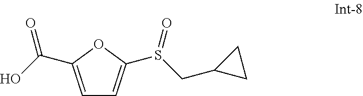 [SMILES: O=C(O)c1ccc(S(=O)CC2CC2)o1]

The title compound was prepared according to the following scheme:

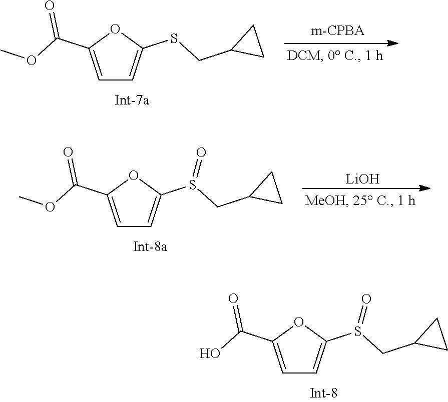

Step 1: Preparation of Methyl 5-(cyclopropylmethylsulfinyl)furan-2-carboxylate (Int-8a)

To a solution of 5-(cyclopropylmethylsulfanyl)furan-2-carboxylate (Int-7a, 382 mg, 1.8 mmol) in DCM (10 mL) was added 3-chloroperoxybenzoic acid (310 mg, 1.8 mmol). After being stirred at 0° C. for 1 h, the mixture was washed with saturated NaHCO₃. The organic layer was dried over Na₂SO₄, filtered and concentrated in vacuo. The residue was purified by flash column (eluting with EtOAc/PE=1/2) to afford methyl 5-(cyclopropylmethylsulfinyl)furan-2-carboxylate as a light yellow oil (Int-8a, 380 mg, 92.5%). MS obsd. (ESI⁺) [(M+H)⁺]: 229.1.

Step 2: Preparation of 5-(cyclopropylmethylsulfinyl)furan-2-carboxylic acid (Int-8)

To a solution of methyl 5-(cyclopropylmethylsulfinyl)furan-2-carboxylate (Int-8a, 380 mg, 1.66 mmol) in a mixed solvent of MeOH (2.5 mL) and THF (2.5 mL) was added LiOH·H₂O (2.5 mL, 2 M). After being stirred at 25° C. for 1 h, most of the solvent was evaporated. The residue was acidified by 3 mL of HCl (2 M) and extracted with EtOAc (20 mL). The organic layer was dried over Na₂SO₄, filtered and concentrated in vacuo to afford 5-(cyclopropylmethylsulfinyl)furan-2-carboxylic acid as a white solid (Int-8, 320 mg, 89.7%). MS obsd. (ESI⁺) [(M+H)⁺]: 215.1.

### Intermediate Int-9

### 5-(cyclopropylmethylsulfonyl)furan-2-carboxylic acid

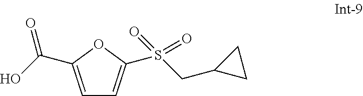 [SMILES: O=C(O)c1ccc(S(=O)(=O)CC2CC2)o1]

The title cornpound was prepared according to the following scheme:

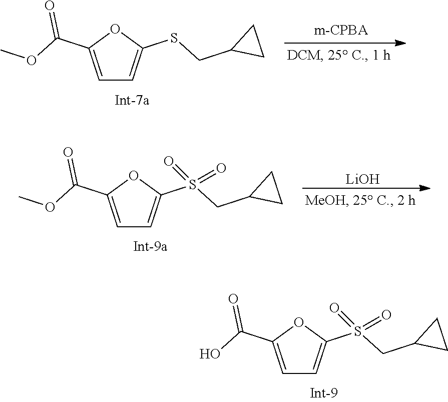 [SMILES: COC(=O)c1ccc(SCC2CC2)o1 ; COC(=O)c1ccc(S(=O)(=O)CC2CC2)o1 ; "LiOH" ; O=C(O)c1ccc(S(=O)(=O)CC2CC2)o1]

Step 1: Preparation of Methyl 5-(cyclopropylmethylsulfonyl)furan-2-carboxylate (Int-9a)

To a solution of methyl 5-(cyclopropylmethylsulfanyl)furan-2-carboxylate (Int-7a, 272 mg, 0.69 mmol) in DCM (10 mL) was added 3-chloroperoxybenzoic acid (358 mg, 2 mmol). After being stirred at 25° C. for 1 h, the mixture was washed with saturated NaHCO₃. The organic layer was dried over Na₂SO₄, filtered and concentrated in vacuo. The residue was purified by flash column (eluting with EtOAc/PE=0 to 20%) to afford methyl 5-(cyclopropylmethylsulfonyl)furan-2-carboxylate as a colorless oil (Int-9a, 110 mg, 59.8%). MS obsd. (ESI⁺) [(M+H)⁺]: 245.1. ¹H NMR (400 MHz, CDCl₃) δ ppm: 7.22-7.25 (m, 2H), 3.94 (d, J=4.2 Hz, 3H), 3.21 (d, J=7.3 Hz, 2H), 1.02-1.18 (m, 1H), 0.56-0.68 (m, 2H), 0.14-0.26 (m, 2H).

Step 2: Preparation of 5-(cyclopropylmethylsulfonyl)furan-2-carboxylic acid (Int-9)

To a solution of methyl 5-(cyclopropylmethylsulfonyl)furan-2-carboxylate (Int-9a, 101 mg, 0.41 mmol) in a mixed solvent of MeOH (9 mL) and water (3 mL) was added LiOH (50 mg, 2 mmol). After being stirred at 25° C. for 2 h, most of the solvent was evaporated. The residue was acidified by HCl (1 M) to pH=2 and extracted with DCM (30 mL×3). The combined organic layer was dried over Na₂SO₄, filtered and concentrated in vacuo to afford 5-(cyclopropylmethylsulfonyl)furan-2-carboxylic acid as an off-white solid (Int-9, 85 mg, 84.8%). MS obsd. (ESI⁺) [(M+H)⁺]: 231.0.

### Intermediate Int-10

### 5-(oxetan-3-ylmethylsulfanyl)furan-2-carboxylic acid

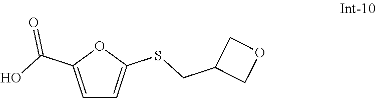 [SMILES: O=C(O)c1ccc(SCC2COC2)o1]

The title compound was prepared according to the following scheme:

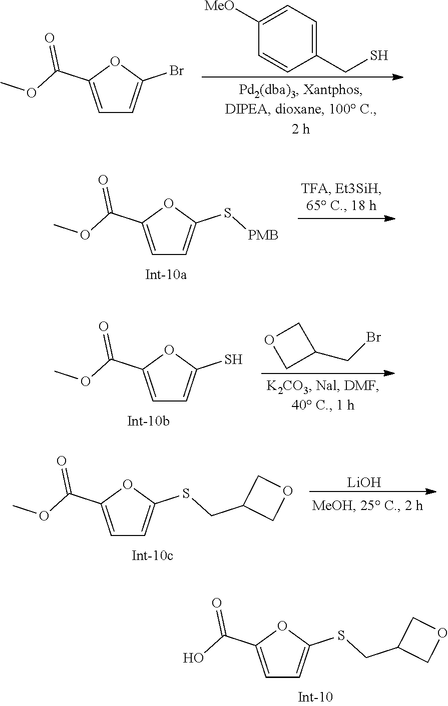

Step 1: Preparation of Methyl 5-[(4-methoxyphenyl)methylsulfanyl]furan-2-carboxylate (Int-10a)

A mixture of methyl 5-bromo-2-furoate (2.24 g, 10.9 mmol), 9,9-dimethyl-4,5-bis(diphenylphosphino)xanthene (631.1 mg, 1.09 mmol), N,N-diisopropylethylamine (3.8 mL, 21.8 mmol), tris(dibenzylideneacetone)dipalladium (0) (499.4 mg, 0.55 mmol) and 4-methoxybenzyl mercaptan (1.68 g, 10.9 mmol) in 1,4-dioxane (50 mL) was stirred at 100° C. for 2 h. The mixture was filtered to remove the solid and the filtrate was concentrated to dryness. The crude product was purified by flash column (eluting with EtOAc/PE=0 to 3%) to afford methyl 5-[(4-methoxyphenyl)methylsulfanyl]furan-2-carboxylate as a yellow oil (Int-10a, 2.7 g, 73.5%). MS obsd. (ESI⁺) [(M+H)⁺]: 279.1.

Step 2: Preparation of Methyl 5-sulfanylfuran-2-carboxylate (Int-10b)

A mixture of methyl 5-[(4-methoxyphenyl)methylsulfanyl]furan-2-carboxylate (Int-10a, 2.7 g, 8.02 mmol), TFA (30 mL, 8.02 mmol) and Et₃SiH (15 mL, 8.02 mmol) was stirred at 65° C. for 16 h. The reaction mixture was concentrated in vacuo to afford methyl 5-sulfanylfuran-2-carboxylate as a brown oil (Int-10b, 1.72 g), which was used for the next step without further purifcation. MS obsd. (ESI⁺) [(M+H)⁺]: 159.1.

Step 3: Preparation of Methyl 5-(oxetan-3-ylmethylsulfanyl)furan-2-carboxylate (Int-10c)

To a solution of methyl 5-sulfanylfuran-2-carboxylate (Int-10b, 450 mg, 2.84 mmol) in DMF (5 mL) were added K₂CO₃ (2.4 g, 17.07 mmol), NaI (42.64 mg, 0.28 mmol) and 3-(bromomethyl)oxetane (0.55 mL, 7.11 mmol). After being stirred at 40° C. for 1 h, the mixture was quenched by water (50 mL) and extracted with EtOAc (30 mL×3). The combined organic layer was concentrated in vacuo. The crude product was purified by flash column (eluting with EtOAc/PE=0 to 10%) to afford methyl 5-(oxetan-3-ylmethylsulfanyl)furan-2-carboxylate as a light yellow oil (Int-10c, 304 mg, 46.8%). MS obsd. (ESI⁺) [(M+H)⁺]: 229.1. ¹H NMR (400 MHz, CDCl₃) δ ppm: 7.13 (d, J=3.4 Hz, 1H), 6.51 (d, J=3.4 Hz, 1H), 4.78 (dd, J=7.4, 6.5 Hz, 2H), 4.36 (t, J=5.9 Hz, 2H), 3.90 (s, 3H), 3.10-3.28 (m, 3H).

Step 4: Preparation of 5-(oxetan-3-ylmethylsulfanyl)furan-2-carboxylic acid (Int-10)

To a solution of methyl 5-(oxetan-3-ylmethylsulfanyl)furan-2-carboxylate (Int-10c, 291 mg, 1.27 mmol) in a mixed solvent of MeOH (15 mL) and water (5 mL) was added LiOH (91.6 mg, 3.8 mmol). After being stirred at 25° C. for 2 h, most of the solvent was evaporated. The residue was acidified by HCl (2 M) to pH=5 and extracted with EtOAc (10 mL×3). The combined organic layer was dried over Na₂SO₄, filtered and concentrated in vacuo to afford 5-(oxetan-3-ylmethylsulfanyl)furan-2-carboxylic acid as a light yellow oil (Int-10, 254 mg, 93%). MS obsd. (ESI⁺) [(M+H)⁺]: 215.0.

### Intermediate Int-11

### 5-(ethylsulfinylmethyl)furan-2-carboxylic acid

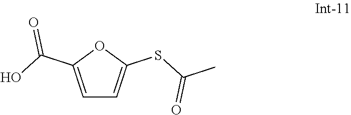

The title compound was prepared according to the following scheme:

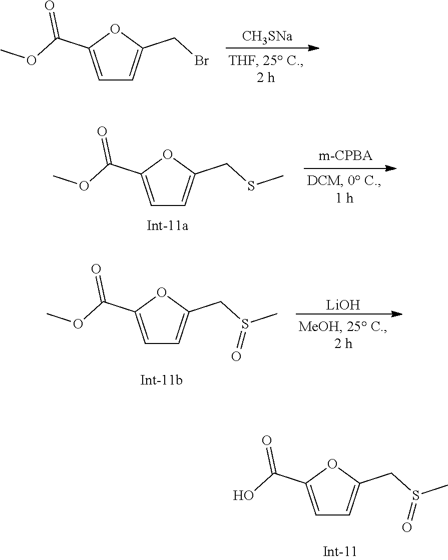

Step 1: Preparation of Methyl 5-(methylsulfanylmethyl)furan-2-carboxylate (Int-11a)

To a solution of methyl 5-(bromomethyl)furan-2-carboxylate (500 mg, 2.28 mmol) in THF (10 mL) was added CH₃SNa (576.09 mg, 6.85 mmol). After being stirred at 25° C. for 2 h, the mixture was extracted with EtOAc (20 mL×3). The organic layer was dried over Na₂SO₄ and concentrated in vacuo to give methyl 5-(methylsulfanylmethyl)furan-2-carboxylate as a yellow oil (Int-11a, 350 mg, 82.3%), which was used for the next step without further purification. MS obsd. (ESI⁺) [(M+H)⁺]: 187.1.

Step 2: Preparation of Methyl 5-(methylsulfinylmethyl)furan-2-carboxylate (Int-11b)

To a solution of methyl 5-(methylsulfanylmethyl)furan-2-carboxylate (Int-11a, 350 mg, 1.88 mmol) in DCM (5 mL) was added m-CPBA (323.26 mg, 1.88 mmol). After being stirred at 0° C. for 1 h, the reaction was quenched with saturated NaHCO₃ (10 mL). The organic phase was separated, dried over Na₂SO₄, filtered and concentrate to give a crude product which was purified by flash column (eluting with DCM/MeOH=94/6) to give methyl 5-(methylsulfinylmethyl)furan-2-carboxylate as a yellow oil (Int-11b, 270 mg, 71.0%). MS obsd. (ESI⁺) [(M+H)⁺]: 203.1.

Step 3: Preparation of 5-(methylsulfnylmethyl)furan-2-carboxylic acid (Int-11)

To a solution of methyl 5-(methylsulfinylmethyl)furan-2-carboxylate (Int-11b, 270 mg, 1.34 mmol) in a mixed solvent of MeOH (6 mL) and water (6 mL) was added LiOH (374.23 mg, 6.68 mmol) at 25° C. After being stirred for 2 h, the mixture was concentrated to remove the MeOH. The residue was acidified to pH=2 with 1M HCl and extracted with DCM (40 mL×3). The organic layer was dried over anhydrous Na₂SO₄ and concentrated in vacuo. The crude product was then purified by flash column (eluting with MeOH/DCM=10%) to afford 5-(methylsulfinylmethyl)furan-2-carboxylic acid as a yellow oil (Int-11, 210 mg, 83.6%). MS obsd. (ESI⁺) [(M+H)⁺]: 189.1.

### Intermediate Int-12

### 5-(sulfamoylmethyl)furan-2-carboxylic acid

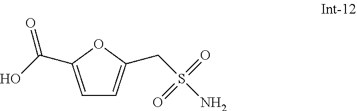 [SMILES: NS(=O)(=O)Cc1ccc(C(=O)O)o1]

The title compound was prepared according to the following scheme:

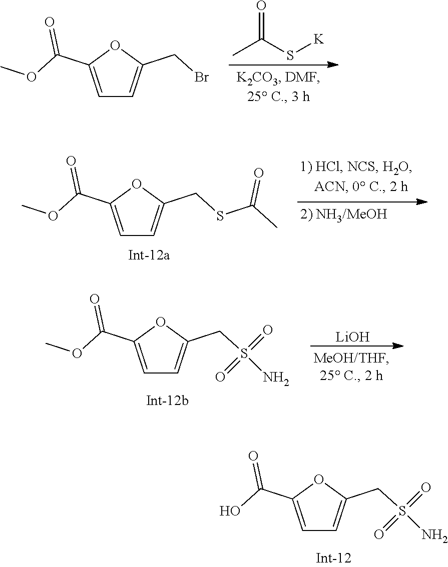

Step 1: Preparation of Methyl 5-(acetylsulfanylmethyl)furan-2-carboxylate (Int-12a)

A mixture of methyl 5-(bromomethyl)furan-2-carboxylate (1.1 g, 5 mmol) and acetylsulfanylpotassium (1.14 g, 10 mmol) in DMF (30 mL) was stirred at 25° C. for 3 h. The solvent was evaporated and the residue was purified by flash column (eluting with EtOAc/PE=1/12) to afford methyl 5-(acetylsulfanylmethyl)furan-2-carboxylate as a yellow oil (Int-12a, 550 mg, 51.3%). MS obsd. (ESI⁺) [(M+H)⁺]: 215.1.

Step 2: Preparation of Methyl 5-(sulfamoylmethyl)furan-2-carboxylate (Int-12b)

To a solution of HCl/H₂O (2 M, 4.18 mL, 8.36 mmol) and ACN (4 mL) was added N-chlorosuccinimide (1.37 g, 10.3 mmol) followed by methyl 5-(acetylsulfanylmethyl)furan-2-carboxylate (Int-12a, 550 mg, 2.57 mmol) at 0° C. After being treated with NH₃/MeOH (0.3 M, 20 mL) at 0° C. for further 2 h, the solvent was evaporated and the residue was separated by EtOAc (20 mL) and brine (30 mL). The organic layer was separated, dried over Na₂SO₄, filtered and concentrated to afford methyl 5-(sulfamoylmethyl)furan-2-carboxylate as a yellow oil (Int-12b, 230 mg, 40.9%). MS obsd. (ESI⁺) [(M+Na)⁺]: 242.0.

Step 3: Preparation of 5-(sulfamoylmethyl)furan-2-carboxylic acid (Int-12)

LiOH·H₂O (10 mL, 20 mmol) in water (10 mL) was added into a solution of methyl 5-(sulfamoylmethyl)furan-2-carboxylate (Int-12b, 250 mg, 1.14 mmol) in a mixed solvent of methanol (10 mL) and THF (10 mL). After being stirred at 25° C. for 2 h, the mixture was acidified by 1 M HCl to pH=5. The solvent was evaporated, and the residue was separated by EtOAc (30 mL) and brine (30 mL). The organic layer was dried over Na₂SO₄, filtered and concentrated in vacuo to afford 5-(sulfamoylmethyl)furan-2-carboxylic acid as a light yellow solid (Int-12, 170 mg, 72.7%). MS obsd. (ESI⁺) [(M+NH₄)⁺]: 223.1.

### Intermediate Int-13

### 2-cyclopropylsulfonylpyridine-4-carboxylic acid

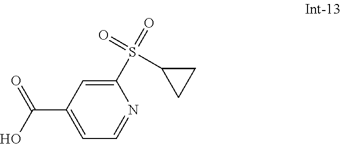 [SMILES: O=C(O)c1ccnc(S(=O)(=O)C2CC2)c1]

The title compound was prepared in analogy to the procedure described for the preparation of 5-methylsulfonylfuran-2-carboxylic acid (Int-3), by using 2-bromoisonicotinic acid instead of methyl 5-bromo-2-furoate and cyclopropylsulfinyloxysodium instead of methylsulfinyloxysodium. MS obsd. (ESI⁺) [(M+H)⁺]: 228.1.

### Intermediate Int-14

### 5-(N-benzyloxycarbonyl-S-methyl-sulfonimidoyl)furan-2-carboxylic acid

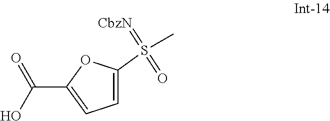

The title compound was prepared according to the following scheme:

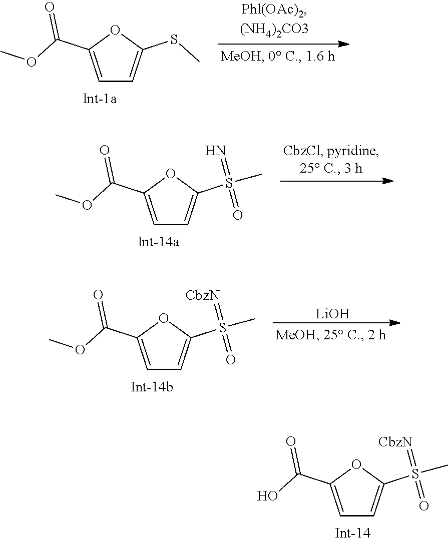

Step 1: Preparation of Methyl 5-(methylsulfonimidoyl)furan-2-carboxylate (Int-14a)

To a solution of methyl 5-methylsulfanylfuran-2-carboxylate (20.1 g, 116.72 mmol) in MeOH (502 mL) were added (NH₄)₂CO₃ (19.07 g, 198.43 mmol) and (diacetoxyiodo)benzene (93.99 g, 291.81 mmol). After being stirred at 25° C. for 1.6 h, the reaction mixture was concentrated in vacuo to remove MeOH. The residue was diluted with DCM (450 mL), washed with water (50 mL×2) and brine (30 mL×3), dried over Na₂SO₄ and concentrated in vacuo to give methyl 5-(methylsulfonimidoyl)furan-2-carboxylate as a colorless liquid (Int-14a, 56.5 g, 78.6%), which was used in the next step without further purification. MS obsd. (ESI⁺) [(M+H)⁺]: 204.1.

Step 2: Preparation of Methyl 5-(N-benzyloxycarbonyl-S-methyl-sulfonimidoyl)furan-2-carboxylate (Int-14b)

To a solution of methyl 5-(methylsulfonimidoyl)furan-2-carboxylate (Int-14a, 56.5 g, 91.75 mmol) in DCM (280 mL) were added pyridine (14.84 mL, 183.5 mmol) and benzyl chloroformate (23.48 g, 137.62 mmol). After being stirred at 25° C. for 3 h, the reaction mixture was washed with HCl (1 M, 15 mL×5) and brine (15 mL×3), dried over Na₂SO₄ and concentrated in vacuo. The crude product was then purified by flash column (eluting with EtOAc/PE=47%) to give methyl 5-(N-benzyloxycarbonyl-S-methyl-sulfonimidoyl)furan-2-carboxylate as a white solid (Int-14b, 22.5 g, 72.7%). MS obsd. (ESI⁺) [(M+H)⁺]: 338.1.

Step 2: Preparation of 5-(N-benzyloxycarbonyl-S-methyl-sulfonimidoyl)furan-2-carboxylic acid (Int-14)

To a solution of methyl 5-(N-benzyloxycarbonyl-S-methyl-sulfonimidoyl)furan-2-carboxylate (Int-14b, 150.0 mg, 0.440 mmol) in a mixed solvent of MeOH (5 mL) and water (5 mL) was added LiOH (124.63 mg, 2.22 mmol). After being stirred at 25° C. for 2 h, the mixture was concentrated in vacuo to remove the MeOH. The residue was acidified to pH=2 with 1 M HCl and extracted with DCM (40 mL×3). The organic layer was dried over Na₂SO₄ and concentrated in vacuo to afford 5-(N-benzyloxycarbonyl-S-methyl-sulfonimidoyl)furan-2-carboxylic acid as a yellow oil (Int-14, 110 mg, 76.52%). MS obsd. (ESI⁺) [(M+H)⁺]: 324.0.

### Intermediate Int-15

### 5-(methylsulfanylmethyl)furan-2-carboxylic acid

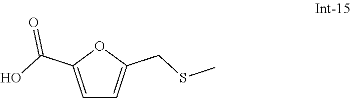

The title compound was prepared according to the following scheme:

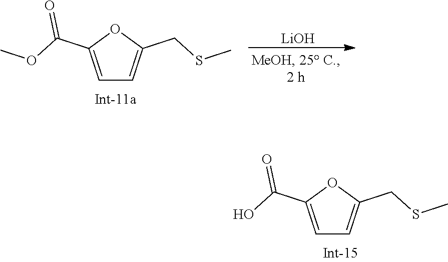

To a solution of methyl 5-(methylsulfanylmethyl)furan-2-carboxylate (Int-11a, 3.8 g, 18.79 mmol) in a mixed solvent of MeOH (20 mL) and water (15 mL) was added LiOH (5.27 g, 93.95 mmol) at 25° C. After being stirred for 2 h, the mixture was concentrated to remove the MeOH. The residue was acidified to pH=2 with 1 M HCl and extracted with DCM (40 mL×3). The organic layer was dried over anhydrous Na₂SO₄ and concentrated in vacuo. The crude product was then purified by preparative HPLC to afford 5-(methylsulfanylmethyl)furan-2-carboxylic acid as a yellow solid (Int-15, 2.6 g, 73.8%). MS obsd. (ESI⁺) [(M+H)⁺]: 173.1.

### Example 1

### N-[3-(5-chloro-1,3-benzoxazol-2-yl)-1-bicyclo[1.1.1]pentanyl]-5-methylsulfonyl-furan-2-carboxamide

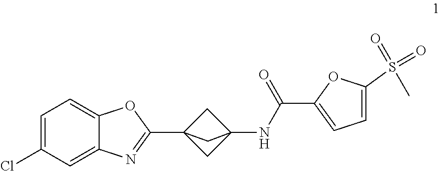 [SMILES: CS(=O)(=O)c1ccc(C(=O)NC23CC(c4nc5cc(Cl)ccc5o4)(C2)C3)o1]

The title compound was prepared according to the following scheme:

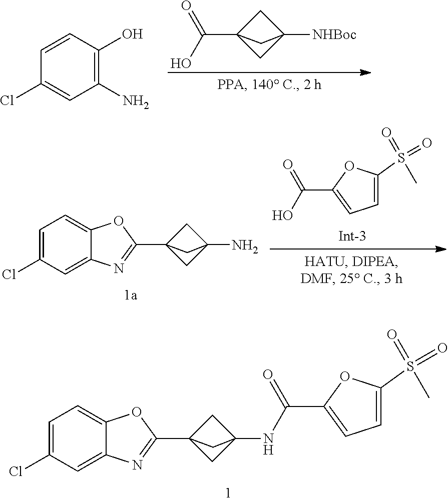 [SMILES: Nc1cc(Cl)ccc1O ; CC(C)(C)OC(=O)NC12CC(C(=O)O)(C1)C2 ; NC12CC(c3nc4cc(Cl)ccc4o3)(C1)C2 ; CS(=O)(=O)c1ccc(C(=O)O)o1 ; CS(=O)(=O)c1ccc(C(=O)NC23CC(c4nc5cc(Cl)ccc5o4)(C2)C3)o1]

Step 1: Preparation of 1-(5-chloro-1,3-benzoxazol-2-yl)bicyclo[1.1.1]pentan-3-amine (1a)

The polyphosphoric acid (5.28 g, 22.01 mmol) in a seal tube was heated with stirring at 110° C. for 10 min, then a mixture of 2-amino-4-chlorophenol (1.58 g, 11.01 mmol) and 3-(tert-butoxycarbonylamino)bicyclo[1.1.1]pentane-1-carboxylic acid (2.5 g, 11.01 mmol) was added. The resulting mixture was heated with stirring at 125° C. for 1 h. After being poured into ice-water (300 mL) and adjusted pH to 8 by NH₄OH, the resulting mixture was extracted with DCM (300 mL×3). The organic layer was dried over Na₂SO₄ and concentrated in vacuo to afford 1-(5-chloro-1,3-benzoxazol-2-yl)bicyclo[1.1.1]pentan-3-amine as a light brown solid (2.32 g, 89.8%). MS obsd. (ESI⁺) [(M+H)⁺]: 235.1.

Step 2: Preparation of N-[3-(5-chloro-1,3-benzoxazol-2-yl)-1-bicyclo[1.1.1]pentanyl]-5-methylsulfonyl-furan-2-carboxamide (Example 1)

A mixture of 5-methylsulfonylfuran-2-carboxylic acid (Int-3, 127.63 mg, 0.67 mmol), HATU (364.55 mg, 0.96 mmol) and DIPEA (413.03 mg, 3.2 mmol) in DCM (6 mL) was stirred at 25° C. for 5 min, followed by addition of 1-(5-chloro-1,3-benzoxazol-2-yl)bicyclo[1.1.1]pentan-3-amine (1a, 150 mg, 0.64 mmol). After being stirred at 25° C. for 16 h, the reaction was quenched by water and extracted with DCM (150 mL). The organic layer was washed with water (50 mL×3) and brine (50 mL×3), dried over MgSO₄ and concentrated in vacuo. The residue was purified by preparative HPLC to afford N-[3-(5-chloro-1,3-benzoxazol-2-yl)-1-bicyclo[1.1.1]pentanyl]-5-methylsulfonyl-furan-2-carboxamide as a light yellow solid (246.8 mg, 93.7%). MS obsd. (ESI⁺) [(M+H)⁺]: 407.1. ¹H NMR (400 MHz, DMSO-d₆) δ ppm: 9.53 (s, 1H), 7.84 (d, J=2.0 Hz, 1H), 7.77 (d, J=8.7 Hz, 1H), 7.46 (dd, J=8.7, 2.1 Hz, 1H), 7.40 (d, J=3.7 Hz, 1H), 7.30 (d, J=3.7 Hz, 1H), 3.37-3.44 (m, 3H), 2.62 (s, 6H).

### Example 2

### Example 2-a, Example 2-b

### N-[3-(5-chloro-1,3-benzoxazol-2-yl)-1-bicyclo[1.1.1]pentanyl]-5-methylsulfinyl-furan-2-carboxamide

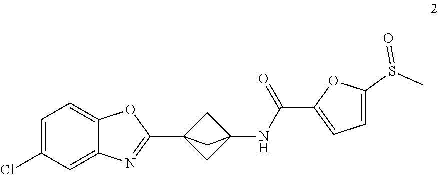 [SMILES: CS(=O)c1ccc(C(=O)NC23CC(c4nc5cc(Cl)ccc5o4)(C2)C3)o1]

N-[3-(5-chloro-1,3-benzoxazol-2-yl)-1-bicyclo[1.1.1]pentanyl]-5-methylsulfinyl-furan-2-carboxamide (Example 2) was prepared in analogy to the procedure described for the preparation of Example 1, by using 5-methylsulfinylfuran-2-carboxylic acid (Int-2) instead of 5-methylsulfonylfuran-2-carboxylic acid (Int-3). MS obsd. (ESI⁺) [(M+H)⁺]: 391.2. ¹H NMR (400 MHz, DMSO-d₆) δ ppm: 9.48 (s, 1H), 7.83 (d, J=2.08 Hz, 1H), 7.76 (d, J=8.68 Hz, 1H), 7.45 (dd, J=2.20, 8.68 Hz, 1H), 7.22-7.32 (m, 2H), 3.05 (s, 3H), 2.61 (s, 6H).

The two enantiomers (Example 2-a, Example 2-b) were obtained through SFC [Instrument: SFC 80; Column: IF, 250×20 mm I.D., 5 μm; Mobile phase: A for CO₂ and B for Methanol (0.1% NH₄OH); Gradient: B 30%; Flow rate: 45 mL/min; Back pressure: 100 bar; Column temperature: 40° C.] chiral separation of N-[3-(5-chloro-1,3-benzoxazol-2-yl)-1-bicyclo[1.1.1]pentanyl]-5-methylsulfinyl-furan-2-carboxamide (Example 2).

### N-[3-(5-chloro-1,3-benzoxazol-2-yl)-1-bicyclo[1.1.1]pentanyl]-5-[(S)-methylsulfinyl]furan-2-carboxamide

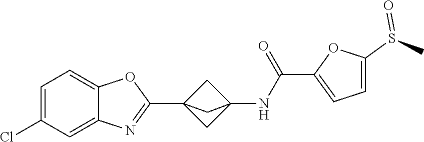 [SMILES: C[S@](=O)c1ccc(C(=O)NC23CC(c4nc5cc(Cl)ccc5o4)(C2)C3)o1]

### N-[3-(5-chloro-1,3-benzoxazol-2-yl)-1-bicyclo[1.1.1]pentanyl]-5-[(R)-methylsulfinyl]furan-2-carboxamide

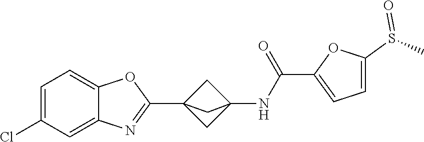 [SMILES: C[S@@](=O)c1ccc(C(=O)NC23CC(c4nc5cc(Cl)ccc5o4)(C2)C3)o1]

Example 2-a: [α]²⁰_{D}=+14.273 (C=0.1, MeOH). MS obsd. (ESI⁺) [(M+H)⁺]: 391.2. ¹H NMR (400 MHz, DMSO-d₆) δ ppm: 9.48 (s, 1H), 7.84 (d, J=2.1 Hz, 1H), 7.77 (d, J=8.8 Hz, 1H), 7.46 (dd, J=8.7, 2.2 Hz, 1H), 7.25-7.30 (m, 2H), 3.05 (s, 3H), 2.61 (s, 6H).

Example 2-b: [α]²⁰_{D}=−12.248 (C=0.1, MeOH). MS obsd. (ESI⁺) [(M+H)⁺]: 391.2. ¹H NMR (400 MHz, DMSO-d₆) δ ppm: 9.48 (s, 1H), 7.84 (d, J=2.0 Hz, 1H), 7.77 (d, J=8.7 Hz, 1H), 7.46 (dd, J=8.7, 2.1 Hz, 1H), 7.24-7.30 (m, 2H), 3.05 (s, 3H), 2.61 (s, 6H).

### Example 3

### N-[3-(5-chloro-1,3-benzoxazol-2-yl)-1-bicyclo[1.1.1]pentanyl]-5-methylsulfanyl-furan-2-carboxamide

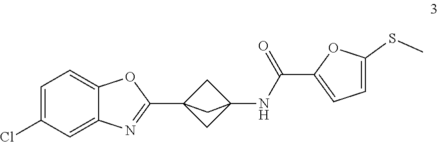 [SMILES: CSc1ccc(C(=O)NC23CC(c4nc5cc(Cl)ccc5o4)(C2)C3)o1]

The title compound was prepared in analogy to the procedure described for the preparation of Example 1, by using 5-methylsulfanylfuran-2-carboxylic acid (Int-1) instead of 5-methylsulfonylfuran-2-carboxylic acid (Int-3). The product was purified by preparative HPLC to afford Example 3 as a white solid. MS obsd. (ESI⁺) [(M+H)⁺]: 375.1. ¹H NMR (400 MHz, DMSO-d₆) δ ppm: 9.12 (s, 1H), 7.83 (d, J=2.1 Hz, 1H), 7.76 (d, J=8.7 Hz, 1H), 7.45 (dd, J=8.7, 2.1 Hz, 1H), 7.12 (d, J=3.4 Hz, 1H), 6.60 (d, J=3.5 Hz, 1H), 2.56-2.60 (m, 6H), 2.52-2.54 (m, 3H).

### Example 4

### N-[3-(5-chloro-1,3-benzoxazol-2-yl)-1-bicyclo[1.1.1]pentanyl]-5-cyclopropylsulfinyl-furan-2-carboxamide

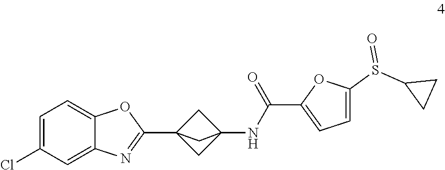 [SMILES: O=C(NC12CC(c3nc4cc(Cl)ccc4o3)(C1)C2)c1ccc(S(=O)C2CC2)o1]

The title compound was prepared in analogy to the procedure described for the preparation of Example 1, by using 5-cyclopropylsulfinylfuran-2-carboxylic acid (Int-5) instead of 5-methylsulfonylfuran-2-carboxylic acid (Int-3). The product was purified by preparative HPLC to afford Example 4 as an off-white solid. MS obsd. (ESI⁺) [(M+H)⁺]: 417.1. ¹H NMR (400 MHz, CDCl₃) δ ppm: 7.69 (d, J=1.83 Hz, 1H), 7.44 (d, J=8.68 Hz, 1H), 7.33 (dd, J=2.02, 8.62 Hz, 1H), 7.22 (d, J=3.55 Hz, 1H), 7.15 (br s, 1H), 6.97 (d, J=3.55 Hz, 1H), 2.76 (s, 6H), 2.71 (br dd, J=3.48, 7.64 Hz, 1H), 1.36-1.47 (m, 1H), 1.18-1.25 (m, 1H), 1.05-1.14 (m, 1H), 0.92-1.03 (m, 1H).

### Example 5

### N-[3-(5-chloro-1,3-benzoxazol-2-yl)-1-bicyclo[1.1.1]pentanyl]-5-cyclopropylsulfonyl-furan-2-carboxamide

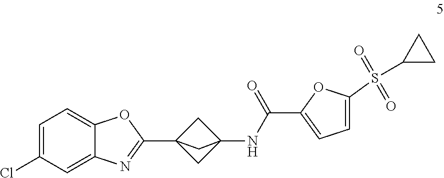 [SMILES: O=C(NC12CC(c3nc4cc(Cl)ccc4o3)(C1)C2)c1ccc(S(=O)(=O)C2CC2)o1]

The title compound was prepared in analogy to the procedure described for the preparation of Example 1, by using 5-cyclopropylsulfonylfuran-2-carboxylic acid (Int-6) instead of 5-methylsulfonylfuran-2-carboxylic acid (Int-3). The product was purified by preparative HPLC to afford Example 5 as a white solid. MS obsd. (ESI⁺) [(M+H)⁺]: 433.0. ¹H NMR (400 MHz, DMSO-d₆) δ ppm: 9.54 (s, 1H), 7.83 (d, J=2.08 Hz, 1H), 7.76 (d, J=8.80 Hz, 1H), 7.45 (dd, J=2.08, 8.68 Hz, 1H), 7.39 (d, J=3.67 Hz, 1H), 7.32 (d, J=3.67 Hz, 1H), 2.92-3.02 (m, 1 H), 2.62 (s, 6H), 1.21-1.28 (m, 2H), 1.13-1.20 (m, 2H).

### Example 6

### N-[3-(5-chloro-1,3-benzoxazol-2-yl)-1-bicyclo[1.1.1]pentanyl]-5-(cyclopropylmethylsulfinyl)furan-2-carboxamide

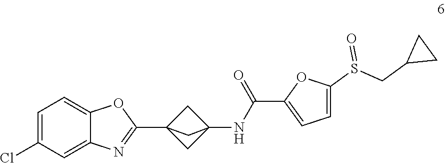 [SMILES: O=C(NC12CC(c3nc4cc(Cl)ccc4o3)(C1)C2)c1ccc(S(=O)CC2CC2)o1]

The title compound was prepared in analogy to the procedure described for the preparation of Example 1, by using 5-(cyclopropylmethylsulfinyl)furan-2-carboxylic acid (Int-8) instead of 5-methylsulfonylfuran-2-carboxylic acid (Int-3). The product was purified by preparative HPLC to afford Example 6 as a white solid. MS obsd. (ESI⁺) [(M+H)⁺]: 431.0. ¹H NMR (400 MHz, CD₃OD) δ ppm: 7.67 (d, J=1.96 Hz, 1H), 7.59 (d, J=8.68 Hz, 1H), 7.40 (dd, J=2.14, 8.74 Hz, 1H), 7.25 (s, 2H), 3.34-3.43 (m, 1H), 3.23-3.29 (m, 1H), 2.70 (s, 6H), 0.89-1.01 (m, 1 H), 0.65 (dqd, J=5.07, 8.88, 17.67 Hz, 2H), 0.39-0.47 (m, 1H), 0.25-0.34 (m, 1H).

### Example 7

### N-[3-(5-chloro-1,3-benzoxazol-2-yl)-1-bicyclo[1.1.1]pentanyl]-5-(cyclopropylmethylsulfonyl)furan-2-carboxamide

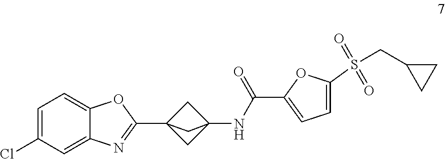 [SMILES: O=C(NC12CC(c3nc4cc(Cl)ccc4o3)(C1)C2)c1ccc(S(=O)(=O)CC2CC2)o1]

The title compound was prepared in analogy to the procedure described for the preparation of Example 1, by using 5-(cyclopropylmethylsulfonyl)furan-2-carboxylic acid (Int-9) instead of 5-methylsulfonylfuran-2-carboxylic acid (Int-3). The product was purified by preparative HPLC to afford Example 7 as a white solid. MS obsd. (ESI⁺) [(M+H)⁺]: 447.1. ¹H NMR (400 MHz, DMSO-d₆) δ ppm: 9.52 (br s, 1H), 7.83 (d, J=2.08 Hz, 1H), 7.76 (d, J=8.68 Hz, 1H), 7.45 (dd, J=2.20, 8.68 Hz, 1H), 7.42 (d, J=3.67 Hz, 1H), 7.32 (d, J=3.79 Hz, 1H), 3.44 (d, J=7.09 Hz, 2H), 2.62 (s, 6H), 0.88-1.00 (m, 1H), 0.46-0.55 (m, 2H), 0.12-0.19 (m, 2H).

### Example 8

### 5-bromo-N-[3-(5-chloro-1,3-benzoxazol-2-yl)-1-bicyclo[1.1.1]pentanyl]furan-2-carboxamide

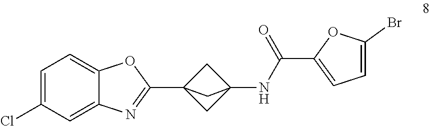 [SMILES: O=C(NC12CC(c3nc4cc(Cl)ccc4o3)(C1)C2)c1ccc(Br)o1]

The title compound was prepared in analogy to the procedure described for the preparation of Example 1, by using 5-bromofuran-2-carboxylic acid instead of 5-methylsulfonylfuran-2-carboxylic acid (Int-3). The product was purified by preparative HPLC to afford Example 8 as a white solid. MS obsd. (ESI⁺) [(M+H)⁺]: 407.0. ¹H NMR (400 MHz, DMSO-d₆) δ ppm: 9.21 (s, 1 H), 7.82 (d, J=2.1 Hz, 1H), 7.76 (d, J=8.7 Hz, 1H), 7.44 (dd, J=8.7, 2.2 Hz, 1H), 7.16 (d, J 10=3.5 Hz, 1H), 6.76 (d, J=3.5 Hz, 1H), 2.57 (s, 6H).

### Example 9

### N-[3-(5-chloro-1,3-benzoxazol-2-yl)-1-bicyclo[1.1.1]pentanyl]-5-methylsulfonyl-thiophene-2-carboxamide

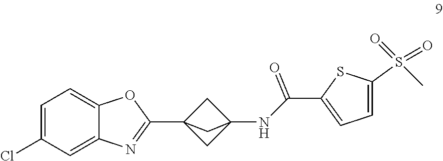 [SMILES: CS(=O)(=O)c1ccc(C(=O)NC23CC(c4nc5cc(Cl)ccc5o4)(C2)C3)s1]

The title compound was prepared in analogy to the procedure described for the preparation of Example 1, by using 5-methylsulfonylthiophene-2-carboxylic acid instead of 5-methylsulfonylfuran-2-carboxylic acid (Int-3). The product was purified by preparative HPLC to afford Example 9 as a white solid. MS obsd. (ESI⁺) [(M+H)⁺]: 423.0. ¹H NMR (400 MHz, DMSO-d₆) δ ppm: 9.63 (s, 1H), 7.76-7.86 (m, 4H), 7.46 (dd, J=8.7, 2.1 Hz, 1H), 3.33 (s, 3 H), 2.62 (s, 6H).

### Example 10

### N-[3-(5-chloro-1,3-benzoxazol-2-yl)-1-bicyclo[1.1.1]pentanyl]-5-sulfamoyl-furan-2-carboxamide

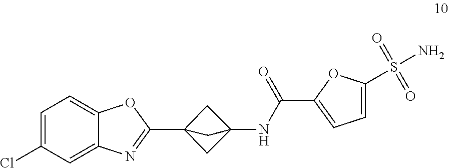 [SMILES: NS(=O)(=O)c1ccc(C(=O)NC23CC(c4nc5cc(Cl)ccc5o4)(C2)C3)o1]

The title compound was prepared in analogy to the procedure described for the preparation of Example 1, by using 5-sulfamoylfuran-2-carboxylic acid instead of 5-methylsulfonylfuran-2-carboxylic acid (Int-3). The product was purified by preparative HPLC to afford Example 10 as a white solid. MS obsd. (ESI⁺) [(M+H)⁺]: 408.0. ¹H NMR (400 MHz, DMSO-d₆) δ ppm: 9.36 (s, 1H), 7.92 (s, 2H), 7.83 (d, J=2.0 Hz, 1H), 7.76 (d, J=8.7 Hz, 1H), 7.45 (dd, J=8.7, 2.1 Hz, 1H), 7.25 (d, J=3.5 Hz, 1H), 7.07 (d, J=3.5 Hz, 1H), 2.60 (s, 6H).

### Example 11

### N-[3-(5-chloro-1,3-benzoxazol-2-yl)-1-bicyclo[1.1.1]pentanyl]-5-(cyclopropylsulfonimidoyl)furan-2-carboxamide

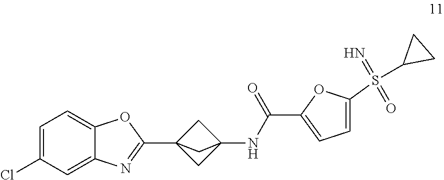 [SMILES: N=S(=O)(c1ccc(C(=O)NC23CC(c4nc5cc(Cl)ccc5o4)(C2)C3)o1)C1CC1]

The title compound was prepared according to the following scheme:

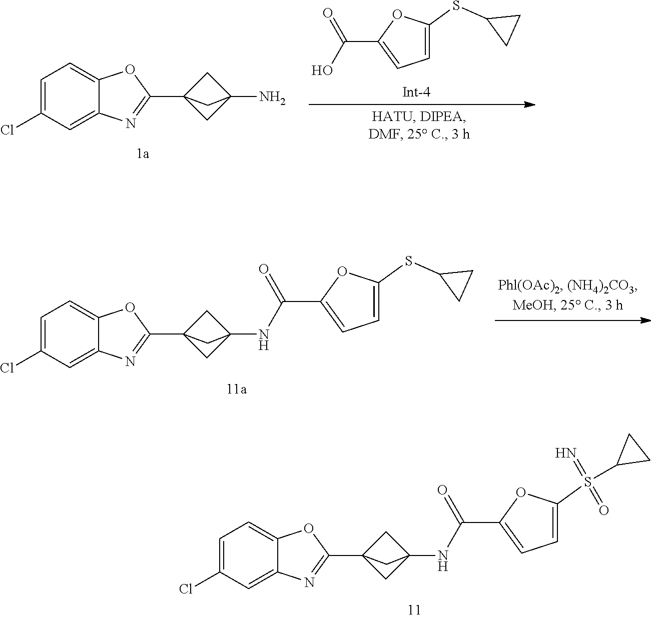 [SMILES: NC12CC(c3nc4cc(Cl)ccc4o3)(C1)C2 ; O=C(O)c1ccc(SC2CC2)o1 ; O=C(NC12CC(c3nc4cc(Cl)ccc4o3)(C1)C2)c1ccc(SC2CC2)o1 ; N=S(=O)(c1ccc(C(=O)NC23CC(c4nc5cc(Cl)ccc5o4)(C2)C3)o1)C1CC1]

Step 1: Preparation of N-[3-(5-chloro-1,3-benzoxazol-2-yl)-1-bicyclo[1.1.1]pentanyl]-5-cyclopropylsulfanyl-furan-2-carboxamide (11a)

The title compound was prepared in analogy to the procedure described for the preparation of Example 1, by using 5-cyclopropylsulfanylfuran-2-carboxylic acid (Int-4) instead of 5-methylsulfonylfuran-2-carboxylic acid (Int-3). The product was purified by preparative HPLC to afford 11a as a light yellow solid. MS obsd. (ESI⁺) [(M+H)⁺]: 401.1.

Step 2: Preparation of N-[3-(5-chloro-1,3-benzoxazol-2-yl)-1-bicyclo[1.1.1]pentanyl]-5-(cyclopropylsulfonimidoyl)furan-2-carboxamide (Example 11)

A mixture of N-[3-(5-chloro-1,3-benzoxazol-2-yl)-3-bicyclo[1.1.1]pentanyl]-5-cyclopropylsulfanyl-furan-2-carboxamide (11a, 53 mg, 0.13 mmol), ammonium carbonate (19.06 mg, 0.2 mmol) and (diacetoxyiodo)benzene (97.94 mg, 0.3 mmol) in MeOH (5 mL) was stirred at 25° C. for 1 h. The resulting mixture was purified by preparative HPLC to afford N-[3-(5-chloro-1,3-benzoxazol-2-yl)-1-bicyclo[1.1.1]pentanyl]-5-(cyclopropylsulfonimidoyl)furan-2-carboxamide as a white solid (Example 11, 42 mg, 72.8%). MS obsd. (ESI⁺) [(M+H)⁺]: 432.1. ¹H NMR (400 MHz, CDCl₃) δ ppm: 7.67 (br d, J=1.83 Hz, 1H), 7.39-7.47 (m, 1H), 7.31 (br dd, J=2.02, 8.62 Hz, 1H), 7.21 (br d, J=3.55 Hz, 1H), 7.17 (br s, 1H), 7.09 (br d, J=3.55 Hz, 1H), 2.61-2.84 (m, 6H), 1.41-1.54 (m, 1H), 1.31 (dt, J=5.01, 11.13 Hz, 1H), 1.13-1.24 (m, 1H), 1.07 (br dd, J=7.34, 12.59 Hz, 1H).

### Example 12

### Example 12-a, Example 12-b

### N-[3-(5-chloro-1,3-benzoxazol-2-yl)-1-bicyclo[1.1.1]pentanyl]-5-(methylsulfonimidoyl)furan-2-carboxamide

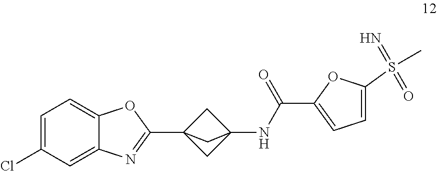 [SMILES: CS(=N)(=O)c1ccc(C(=O)NC23CC(c4nc5cc(Cl)ccc5o4)(C2)C3)o1]

N-[3-(5-chloro-1,3-benzoxazol-2-yl)-1-bicyclo[1.1.1]pentanyl]-5-(methylsulfonimidoyl)furan-2-carboxamide (Example 12) was prepared in analogy to the procedure described for the preparation of Example 11, by using 5-methylsulfanylfuran-2-carboxylic acid (Int-1) instead of 5-cyclopropylsulfanylfuran-2-carboxylic acid (Int-4). The product was purified by preparative HPLC to afford Example 12 as an off-white solid. MS obsd. (ESI⁺) [(M+H)⁺]: 406.1. ¹H NMR (400 MHz, DMSO-d₆) δ ppm: 9.46 (s, 1H), 7.84 (d, J=2.0 Hz, 1H), 7.77 (d, J=8.7 Hz, 1H), 7.46 (dd, J=8.7, 2.2 Hz, 1H), 7.23 (d, J=3.5 Hz, 1H), 7.16 (d, J=3.5 Hz, 1H), 4.87-4.92 (m, 1H), 3.21 (d, J=1.3 Hz, 3H), 2.61 (s, 6H).

The two enantiomers (Example 12-a, Example 12-b) were obtained through SFC [Instrument: SFC 80; Column: IC, 250×30 mm I.D., 5 μm; Mobile phase: A for CO₂ and B for Methanol (0.1% NH₄OH); Gradient: B 30%; Flow rate: 60 mL/min; Back pressure: 100 bar; Column temperature: 40° C.] chiral separation of N-[3-(5-chloro-1,3-benzoxazol-2-yl)-1-bicyclo[1.1.1]pentanyl]-5-(methylsulfonimidoyl)furan-2-carboxamide (Example 12).

### N-[3-(5-chloro-1,3-benzoxazol-2-yl)-1-bicyclo[1.1.1]pentanyl]-5-[(R)-methylsulfonimidoyl]furan-2-carboxamide

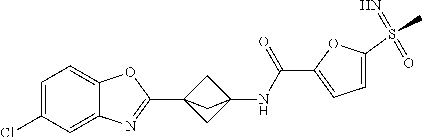 [SMILES: C[S@@](=N)(=O)c1ccc(C(=O)NC23CC(c4nc5cc(Cl)ccc5o4)(C2)C3)o1]

### N-[3-(5-chloro-1,3-benzoxazol-2-yl)-1-bicyclo[1.1.1]pentanyl]-5-[(S)-methylsulfonimidoyl]furan-2-carboxamide

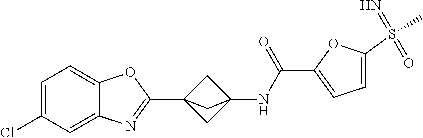 [SMILES: C[S@](=N)(=O)c1ccc(C(=O)NC23CC(c4nc5cc(Cl)ccc5o4)(C2)C3)o1]

Example 12-a: [α]²⁰_{D}=+25.807 (C=0.1, MeOH). MS obsd. (ESI⁺) [(M+H)⁺]: 406.0. ¹H NMR (400 MHz, DMSO-d₆) δ ppm: 9.45 (s, 1H), 7.83 (d, J=2.1 Hz, 1H), 7.76 (d, J=8.7 Hz, 1H), 7.45 (dd, J=8.7, 2.1 Hz, 1H), 7.23 (d, J=3.7 Hz, 1H), 7.16 (d, J=3.5 Hz, 1H), 4.89 (s, 1 H), 3.20 (s, 3H), 2.61 (s, 6H).

Example 12-b: [α]²⁰_{D}=−15.351 (C=0.1, MeOH). MS obsd. (ESI⁺) [(M+H)⁺]: 406.0. ¹H NMR (400 MHz, DMSO-d₆) δ ppm: 9.45 (s, 1H), 7.83 (d, J=2.1 Hz, 1H), 7.76 (d, J=8.8 Hz, 1H), 7.45 (dd, J=8.7, 2.2 Hz, 1H), 7.23 (d, J=3.7 Hz, 1H), 7.16 (d, J=3.7 Hz, 1H), 4.86-4.92 (m, 1H), 3.20 (d, J=1.2 Hz, 3H), 2.61 (s, 6H).

### Example 13

### N-[3-(5-chloro-1,3-benzoxazol-2-yl)-1-bicyclo[1.1.1]pentanyl]-5-(cyclopropylmethylsulfonimidoyl)furan-2-carboxamide

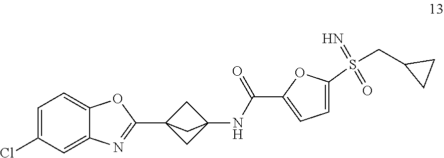 [SMILES: N=S(=O)(CC1CC1)c1ccc(C(=O)NC23CC(c4nc5cc(Cl)ccc5o4)(C2)C3)o1]

The title compound was prepared in analogy to the procedure described for the preparation of Example 11, by using 5-(cyclopropylmethylsulfanyl)furan-2-carboxylic acid (Int-7) instead of 5-cyclopropylsulfanylfuran-2-carboxylic acid (Int-4). The product was purified by preparative HPLC to afford Example 13 as a white solid. MS obsd. (ESI⁺) [(M+H)⁺]: 446.1. ¹H NMR (400 MHz, CD₃OD) δ ppm: 7.68 (d, J=2.08 Hz, 1H), 7.59 (d, J=8.68 Hz, 1H), 7.40 (dd, J=2.08, 8.80 Hz, 1H), 7.26 (q, J=3.63 Hz, 2H), 3.34 (s, 2H), 2.70 (s, 6H), 1.04-1.17 (m, 1H), 0.50-0.63 (m, 2H), 0.09-0.23 (m, 2H).

### Example 14

### N-[3-(5-chloro-1,3-benzoxazol-2-yl)-1-bicyclo[1.1.1]pentanyl]-5-(oxetan-3-ylmethylsulfonyl)furan-2-carboxamide

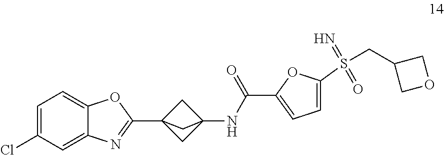 [SMILES: N=S(=O)(CC1COC1)c1ccc(C(=O)NC23CC(c4nc5cc(Cl)ccc5o4)(C2)C3)o1]

The title compound was prepared according to the following scheme:

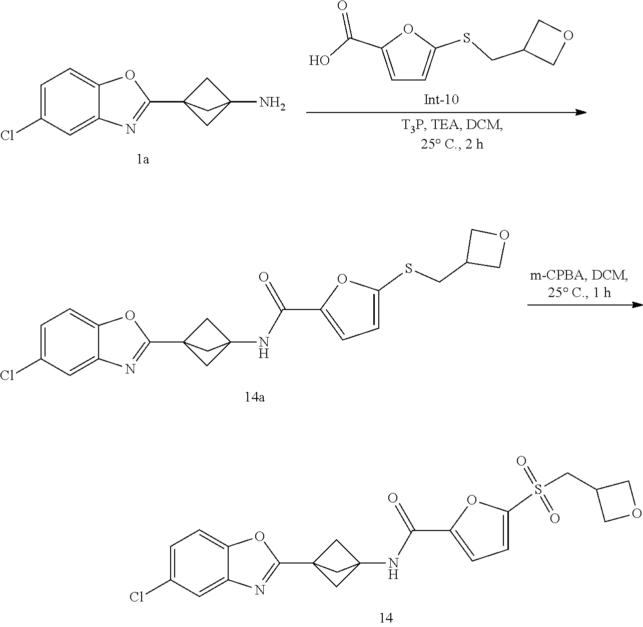 [SMILES: NC12CC(c3nc4cc(Cl)ccc4o3)(C1)C2 ; O=C(O)c1ccc(SCC2COC2)o1 ; O=C(NC12CC(c3nc4cc(Cl)ccc4o3)(C1)C2)c1ccc(SCC2COC2)o1 ; O=C(NC12CC(c3nc4cc(Cl)ccc4o3)(C1)C2)c1ccc(S(=O)(=O)CC2COC2)o1]

Step 1: Preparation of N-[3-(5-chloro-1,3-benzoxazol-2-yl)-3-bicyclo[1.1.1]pentanyl]-5-(oxetan-3-ylmethylsulfanyl)furan-2-carboxamide (14a)

To a solution of 5-(oxetan-3-ylmethylsulfanyl)furan-2-carboxylic acid (Int-10, 241.01 mg, 1.12 mmol) and 1-(5-chloro-1,3-benzoxazol-2-yl)bicyclo[1.1.1]pentan-3-amine (1a, 240 mg, 1.02 mmol) in DCM (30 mL) were added triethylamine (310.45 mg, 3.1 mmol) and T₃P (715.86 mg, 1.1 mmol). After being stirred at 25° C. for 2 h, the mixture was concentrated in vacuo and the residue was purified by flash column (eluting with EtOAc/PE=0 to 100%) to afford N-[3-(5-chloro-1,3-benzoxazol-2-yl)-3-bicyclo[1.1.1]pentanyl]-5-(oxetan-3-ylmethylsulfanyl)furan-2-carboxamide as a yellow solid (14a, 331 mg, 75.11%). MS obsd. (ESI⁺) [(M+H)⁺]: 431.0.

Step 2: Preparation of N-[3-(5-chloro-1,3-benzoxazol-2-yl)-1-bicyclo[1.1.1]pentanyl]-5-(oxetan-3-ylmethylsulfonyl)furan-2-carboxamide (Example 14)

To a solution of N-[3-(5-chloro-1,3-benzoxazol-2-yl)-3-bicyclo[1.1.1]pentanyl]-5-(oxetan-3-ylmethylsulfanyl)furan-2-carboxamide (14a, 50 mg, 0.12 mmol) in DCM (3 mL) was added m-CPBA (100.12 mg, 0.58 mmol). After being stirred at 25° C. for 1 h, the mixture was concentrated in vacuo and the residue was purified by preparative HPLC to afford N-[3-(5-chloro-1,3-benzoxazol-2-yl)-1-bicyclo[1.1.1]pentanyl]-5-(oxetan-3-ylmethylsulfonyl)furan-2-carboxamide as a white solid (Example 14, 37 mg, 68.2%). MS obsd. (ESI⁺) [(M+H)⁺]: 463.0. ¹H NMR (400 MHz, DMSO-d₆) δ ppm: 9.55 (s, 1H), 7.83 (d, J=1.96 Hz, 1H), 7.77 (d, J=8.68 Hz, 1H), 7.45 (dd, J=2.20, 8.68 Hz, 1H), 7.43 (d, J=3.67 Hz, 1H), 7.32 (d, J=3.79 Hz, 1H), 4.59 (dd, J=6.11, 8.07 Hz, 2H), 4.33 (t, J=6.48 Hz, 2H), 3.94 (d, J=7.46 Hz, 2H), 3.35-3.42 (m, 1H), 2.62 (s, 6H).

### Example 15

### N-[3-(5-chloro-1,3-benzoxazol-2-yl)-1-bicyclo[1.1.1]pentanyl]-5-(oxetan-3-ylmethylsulfonimidoyl)furan-2-carboxamide

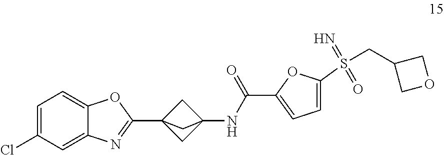 [SMILES: N=S(=O)(CC1COC1)c1ccc(C(=O)NC23CC(c4nc5cc(Cl)ccc5o4)(C2)C3)o1]

The title compound was prepared in analogy to the procedure described for the preparation of Example 11, by using 5-(oxetan-3-ylmethylsulfanyl)furan-2-carboxylic acid (Int-10) instead of 5-cyclopropylsulfanylfuran-2-carboxylic acid (Int-4). The product was purified by preparative HPLC to afford Example 15 as a white solid. MS obsd. (ESI⁺) [(M+H)⁺]: 462.0. ¹H NMR (400 MHz, DMSO-d₆) δ ppm: 9.46 (s, 1H), 7.83 (d, J=1.96 Hz, 1H), 7.77 (d, J=8.68 Hz, 1H), 7.45 (dd, J=2.08, 8.68 Hz, 1H), 7.25 (d, J=3.55 Hz, 1H), 7.18 (d, J=3.55 Hz, 1H), 4.99 (s, 1 H), 4.56 (ddd, J=2.32, 6.02, 8.16 Hz, 2H), 4.25-4.37 (m, 2H), 3.70 (d, J=7.46 Hz, 2H), 3.35-3.42 (m, 1H), 2.62 (s, 6H).

### Example 16

### N-[3-(5-chloro-1,3-benzoxazol-2-yl)-1-bicyclo[1.1.1]pentanyl]-5-(methylsulfonylmethyl)furan-2-carboxamide

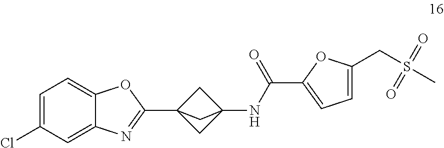 [SMILES: CS(=O)(=O)Cc1ccc(C(=O)NC23CC(c4nc5cc(Cl)ccc5o4)(C2)C3)o1]

The title compound was prepared in analogy to the procedure described for the preparation of Example 1, by using 5-(methylsulfonylmethyl)furan-2-carboxylic acid instead of 5-methylsulfonylfuran-2-carboxylic acid (Int-3). The product was purified by preparative HPLC to afford Example 16 as a white solid. MS obsd. (ESI⁺) [(M+H)⁺]: 421.1. tH NMR (400 MHz, DMSO-d₆) δ ppm: 9.14 (s, 1H), 7.83 (d, J=2.08 Hz, 1H), 7.76 (d, J=8.80 Hz, 1H), 7.45 (dd, J=2.20, 8.68 Hz, 1H), 7.17 (d, J=3.55 Hz), 6.67 (d, J=3.42 Hz, 1H), 4.71 (s, 2H), 3.05 (s, 3H), 2.59 (s, 6H).

### Example 17

### N-[3-(5-chloro-1,3-benzoxazol-2-yl)-1-bicyclo[1.1.1]pentanyl]-5-[(methylsulfonimidoyl)methyl]furan-2-carboxamide

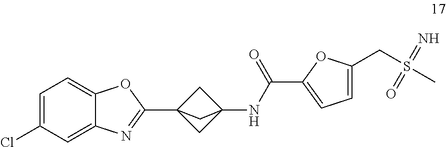 [SMILES: CS(=N)(=O)Cc1ccc(C(=O)NC23CC(c4nc5cc(Cl)ccc5o4)(C2)C3)o1]

The title compound was prepared according to the following scheme:

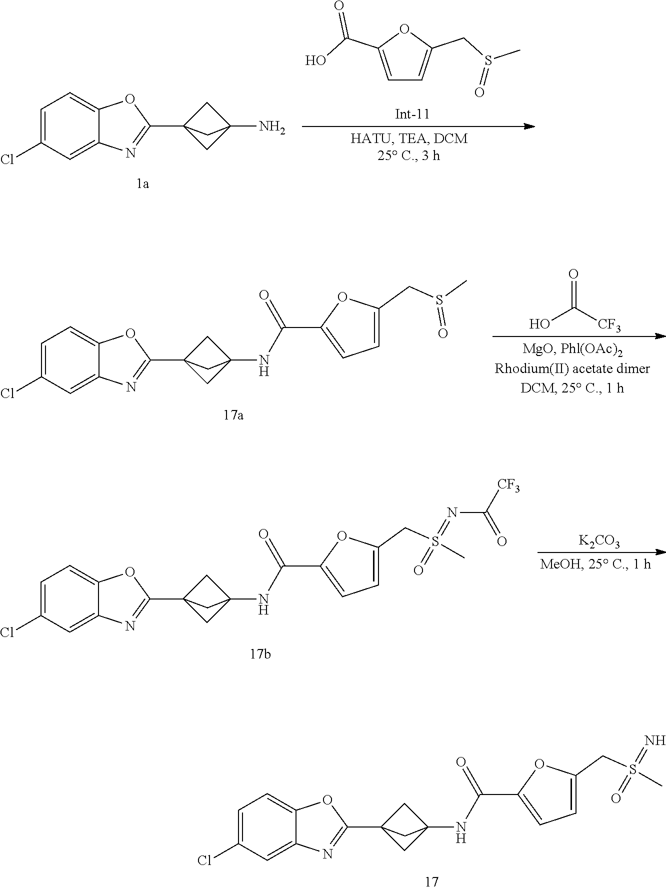

Step 1: Preparation of N-[3-(5-chloro-1,3-benzoxazol-2-yl)-1-bicyclo[1.1.1]pentanyl]-5-(methylsulfinylmethyl)furan-2-carboxamide (17a)

The title compound was prepared in analogy to the procedure described for the preparation of Example 1, by using 5-(methylsulfinylmethyl)furan-2-carboxylic acid (Int-11) instead of 5-methylsulfonylfuran-2-carboxylic acid (Int-3). The product was purified by preparative HPLC to afford 17a as a brown oil. MS obsd. (ESI⁺) [(M+H)⁺]: 404.9.

Step 2: Preparation of N-[3-(5-chloro-1,3-benzoxazol-2-yl)-1-bicyclo[1.1.1]pentanyl]-5-[[S-methyl-N-(2,2,2-trifluoroacetyl)sulfonimidoyl]methyl]furan-2-carboxamide (17b)

To a solution of N-[3-(5-chloro-1,3-benzoxazol-2-yl)-3-bicyclo[1.1.1]pentanyl]-5-(methylsulfinylmethyl)furan-2-carboxamide (17a) (110.0 mg, 0.270 mmol) in DCM (5 mL) were added 2,2,2-trifluoroacetamide (92.14 mg, 0.82 mmol), MgO (43.47 mg, 1.09 mmol), PhI(OAc)₂ (262.45 mg, 0.82 mmol) and Rhodium(II) acetate dimer (12.01 mg, 0.030 mmol). After being stirred at 25° C. for 1 h, the mixture was extracted with EtOAc (15 mL×3). The organic layer was dried over Na₂SO₄ and concentrated in vacuo. The residue was purified by flash column (eluting with DCM/MeOH=24/1) to afford N-[3-(5-chloro-1,3-benzoxazol-2-yl)-1-bicyclo[1.1.1]pentanyl]-5-[[S-methyl-N-(2,2,2-trifluoroacetyl)sulfonimidoyl]methyl]furan-2-carboxamide as a brown oil (17b, 60 mg, 42.8%). MS obsd. (ESI⁺) [(M+H)⁺]: 515.8.

Step 3: Preparation of N-[3-(5-chloro-1,3-benzoxazol-2-yl)-1-bicyclo[1.1.1]pentanyl]-5-[(methylsulfonimidoyl)methyl]furan-2-carboxamide (Example 17)

To a solution of N-[3-(5-chloro-1,3-benzoxazol-2-yl)-1-bicyclo[1.1.1]pentanyl]-5-[[S-methyl-N-(2,2,2-trifluoroacetyl)sulfonimidoyl]methyl]furan-2-carboxamide (17b, 60.0 mg, 0.12 mmol) in MeOH (2 mL) was added K₂CO₃ (48.3 mg, 0.35 mmol). After being stirred at 25° C. for 1 h, the reaction was extracted with DCM (20 mL×3) and with water (10 mL). The organic layer was washed with brine (15 mL), dried over MgSO₄ and concentrated in vacuo. The residue was purified by preparative HPLC to afforded N-[3-(5-chloro-1,3-benzoxazol-2-yl)-1-bicyclo[1.1.1]pentanyl]-5-[(methylsulfonimidoyl)methyl]furan-2-carboxamide as a white solid (Example 17, 25 mg, 48.6%). MS obsd. (ESI⁺) [(M+H)⁺]: 420.0. ¹H NMR (400 MHz, DMSO-d₆) δ ppm: 9.10 (s, 1H), 7.83 (d, J=2.08 Hz, 1H), 7.76 (d, J=8.68 Hz, 1H), 7.45 (dd, J=2.08, 8.68 Hz, 1H), 7.14 (d, J=3.42 Hz, 1H), 6.63 (d, J=3.42 Hz, 1H), 4.50-4.63 (m, 2H), 3.94 (s, 1H), 2.93 (s, 3H), 2.59 (s, 6H).

### Example 18

### N-[3-(5-chloro-1,3-benzoxazol-2-yl)-1-bicyclo[1.1.1]pentanyl]-5-(sulfamoylmethyl)furan-2-carboxamide

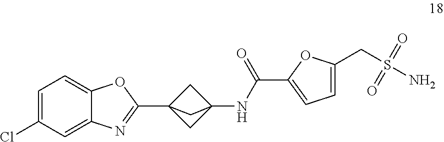 [SMILES: NS(=O)(=O)Cc1ccc(C(=O)NC23CC(c4nc5cc(Cl)ccc5o4)(C2)C3)o1]

The title compound was prepared in analogy to the procedure described for the preparation of Example 1, by using 5-(sulfamoylmethyl)furan-2-carboxylic acid (Int-12) instead of 5-methylsulfonylfuran-2-carboxylic acid (Int-3). The product was purified by preparative HPLC to afford Example 18 as a white solid. MS obsd. (ESI⁺) [(M+H)⁺]: 422.0. ¹H NMR (400 MHz, CD₃OD) δ ppm: 7.67 (d, J=1.96 Hz, 1H), 7.59 (d, J=8.68 Hz, 1H), 7.39 (dd, J=2.02, 8.74 Hz, 1H), 7.13 (d, J=3.55 Hz, 1H), 6.63 (d, J=3.42 Hz, 1H), 4.52 (s, 2H), 2.68 (s, 6H).

### Example 19

### 3-bromo-N-[3-(5-chloro-1,3-benzoxazol-2-yl)-1-bicyclo[1.1.1]pentanyl]benzamide

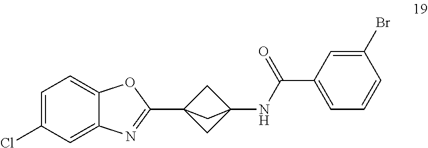 [SMILES: O=C(NC12CC(c3nc4cc(Cl)ccc4o3)(C1)C2)c1cccc(Br)c1]

The title compound was prepared in analogy to the procedure described for the preparation of Example 1, by using 3-bromobenzoic acid instead of 5-methylsulfonylfuran-2-carboxylic acid (Int-3). The product was purified by preparative HPLC to afford Example 19 as a white solid. MS obsd. (ESI⁺) [(M+H)⁺]: 417.0. ¹H NMR (400 MHz, DMSO-d₆) δ ppm: 9.36 (s, 1H), 8.05 (t, J=1.7 Hz, 1H), 7.87 (d, J=7.9 Hz, 1H), 7.83 (d, J=2.0 Hz, 1H), 7.74-7.79 (m, 2H), 7.43-7.49 (m, 2H), 2.61 (s, 6H).

### Example 20

### 4-bromo-N-[3-(5-chloro-1,3-benzoxazol-2-yl)-1-bicyclo[1.1.1]pentanyl]benzamide

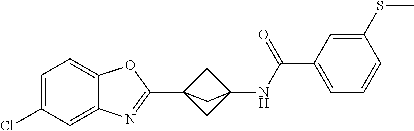 [SMILES: CSc1cccc(C(=O)NC23CC(c4nc5cc(Cl)ccc5o4)(C2)C3)c1]

The title compound was prepared in analogy to the procedure described for the preparation of Example 1, by using 4-bromobenzoic acid instead of 5-methylsulfonylfuran-2-carboxylic acid (Int-3). The product was purified by preparative HPLC to afford Example 20 as a white solid. MS obsd. (ESI⁺) [(M+H)⁺]: 417.1. ¹H NMR (400 MHz, DMSO-d₆) δ ppm: 9.32 (s, 1H), 7.68-7.85 (m, 6H), 7.45 (dd, J=8.7, 2.2 Hz, 1H), 2.60 (s, 6H).

### Example 21

### N-[3-(5-chloro-1,3-benzoxazol-2-yl)-1-bicyclo[1.1.1]pentanyl]-3-methylsulfanyl-benzamide

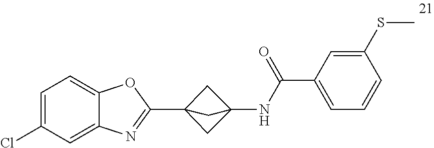 [SMILES: CSc1cccc(C(=O)NC23CC(c4nc5cc(Cl)ccc5o4)(C2)C3)c1]

The title compound was prepared in analogy to the procedure described for the preparation of Example 1, by using 3-methylsulfanylbenzoic acid instead of 5-methylsulfonylfuran-2-carboxylic acid (Int-3). The product was purified by preparative HPLC to afford Example 21 as a white solid. MS obsd. (ESI⁺) [(M+H)⁺]: 385.1. ¹H NMR (400 MHz, DMSO-d₆) δ ppm: 9.27 (s, 1H), 7.83 (d, J=2.1 Hz, 1H), 7.77 (d, J=8.7 Hz, 1H), 7.72 (s, 1H), 7.59-7.65 (m, 1H), 7.39-7.47 (m, 3H), 2.61 (s, 6H), 2.52-2.54 (m, 3H).

### Example 22

### N-[3-(5-chloro-1,3-benzoxazol-2-yl)-1-bicyclo[1.1.1]pentanyl]-3-methylsulfonyl-benzamide

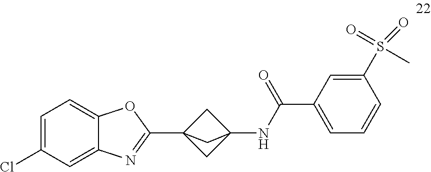 [SMILES: CS(=O)(=O)c1cccc(C(=O)NC23CC(c4nc5cc(Cl)ccc5o4)(C2)C3)c1]

The title compound was prepared in analogy to the procedure described for the preparation of Example 1, by using 3-methylsulfonylbenzoic acid instead of 5-methylsulfonylfuran-2-carboxylic acid (Int-3). The product was purified by preparative HPLC to afford Example 22 as a white solid. MS obsd. (ESI⁺) [(M+H)⁺]: 417.0. ¹H NMR (400 MHz, DMSO-d₆) δ ppm: 9.56 (s, 1H), 8.42 (t, J=1.5 Hz, 1H), 8.20 (d, J=7.9 Hz, 1H), 8.10 (d, J=7.5 Hz, 1H), 7.76-7.85 (m, 3H), 7.46 (dd, J=8.7, 2.1 Hz, 1H), 3.33 (s, 2H), 3.23-3.30 (m, 3H), 2.64 (s, 6H).

### Example 23

### N-[3-(5-chloro-1,3-benzoxazol-2-yl)-1-bicyclo[1.1.1]pentanyl]-2-(trifluoromethyl)pyridine-4-carboxamide

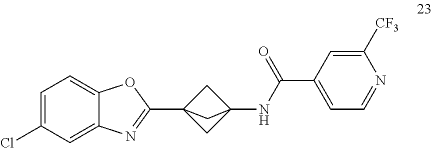 [SMILES: O=C(NC12CC(c3nc4cc(Cl)ccc4o3)(C1)C2)c1ccnc(C(F)(F)F)c1]

The title compound was prepared in analogy to the procedure described for the preparation of Example 1, by using 2-(trifluoromethyl)pyridine-4-carboxylic acid instead of 5-methylsulfonylfuran-2-carboxylic acid (Int-3). The product was purified by preparative HPLC to afford Example 23 as a white solid. MS obsd. (ESI⁺) [(M+H)⁺]: 408.1. ¹H NMR (400 MHz, DMSO-d₆) δ ppm: 9.78 (s, 1H), 8.96 (d, J=5.0 Hz, 1H), 8.28 (s, 1H), 8.11 (d, J=4.9 Hz, 1H), 7.84 (d, J=2.1 Hz, 1H), 7.78 (d, J=8.7 Hz, 1H), 7.46 (dd, J=8.7, 2.2 Hz, 1H), 2.65 (s, 6H).

### Example 24

### N-[3-(5-chloro-1,3-benzoxazol-2-yl)-1-bicyclo[1.1.1]pentanyl]-5-(trifluoromethyl)pyridine-3-carboxamide

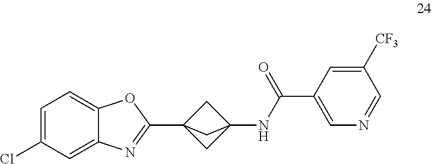 [SMILES: O=C(NC12CC(c3nc4cc(Cl)ccc4o3)(C1)C2)c1cncc(C(F)(F)F)c1]

The title compound was prepared in analogy to the procedure described for the preparation of Example 1, by using 5-(trifluoromethyl)pyridine-3-carboxylic acid instead of 5-methylsulfonylfuran-2-carboxylic acid (Int-3). The product was purified by preparative HPLC to afford Example 24 as a white solid. MS obsd. (ESI⁺) [(M+H)⁺]: 408.1. ¹H NMR (400 MHz, DMSO-d₆) δ ppm: 9.68 (s, 1H), 9.29 (d, J=1.8 Hz, 1H), 9.16 (d, J=1.2 Hz, 1H), 8.60 (s, 1H), 7.84 (d, J=2.2 Hz, 1H), 7.78 (d, J=8.7 Hz, 1H), 7.46 (dd, J=8.7, 2.2 Hz, 1H), 2.65 (s, 6H).

### Example 25

### N-[3-(5-chloro-1,3-benzoxazol-2-yl)-1-bicyclo[1.1.1]pentanyl]-6-(trifluoromethyl)pyridine-3-carboxamide

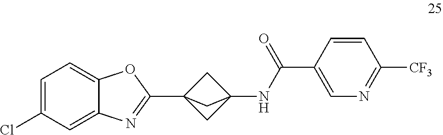 [SMILES: O=C(NC12CC(c3nc4cc(Cl)ccc4o3)(C1)C2)c1ccc(C(F)(F)F)nc1]

The title compound was prepared in analogy to the procedure described for the preparation of Example 1, by using 6-(trifluoromethyl)pyridine-3-carboxylic acid instead of 5-methylsulfonylfuran-2-carboxylic acid (Int-3). The product was purified by preparative HPLC to afford Example 25 as a white solid. MS obsd. (ESI⁺) [(M+H)⁺]: 408.1. ¹H NMR (400 MHz, DMSO-d₆) δ ppm: 9.67 (s, 1H), 9.16 (d, J=1.7 Hz, 1H), 8.48 (dd, J=8.1, 1.9 Hz, 1H), 8.07 (d, J=8.1 Hz, 1H), 7.84 (d, J=2.1 Hz, 1H), 7.77 (d, J=8.7 Hz, 1H), 7.46 (dd, J=8.7, 2.2 Hz, 1 H), 2.63-2.67 (m, 6H).

### Example 26

### N-[3-(5-chloro-1,3-benzoxazol-2-yl)-1-bicyclo[1.1.1]pentanyl]-2-(trifluoromethyl)pyrimidine-5-carboxamide

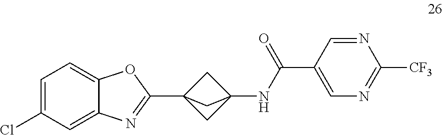 [SMILES: O=C(NC12CC(c3nc4cc(Cl)ccc4o3)(C1)C2)c1cnc(C(F)(F)F)nc1]

The title compound was prepared in analogy to the procedure described for the preparation of Example 1, by using 2-(trifluoromethyl)pyrimidine-5-carboxylic acid instead of 5-methylsulfonylfuran-2-carboxylic acid (Int-3). The product was purified by preparative HPLC to afford Example 26 as a white solid. MS obsd. (ESI⁺) [(M+H)⁺]: 409.1. ¹H NMR (400 MHz, DMSO-d₆) δ ppm: 9.82 (s, 1H), 9.40 (s, 2H), 7.84 (d, J=2.1 Hz, 1H), 7.78 (d, J=8.7 Hz, 1H), 7.46 (dd, J=8.7, 2.1 Hz, 1H), 2.66 (s, 6H).

### Example 27

### N-[3-(5-chloro-1,3-benzoxazol-2-yl)-1-bicyclo[1.1.1]pentanyl]-6-(trifluoromethyl)pyridazine-3-carboxamide

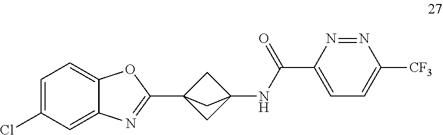 [SMILES: O=C(NC12CC(c3nc4cc(Cl)ccc4o3)(C1)C2)c1ccc(C(F)(F)F)nn1]

The title compound was prepared in analogy to the procedure described for the preparation of Example 1, by using 6-(trifluoromethyl)pyridazine-3-carboxylic acid instead of 5-methylsulfonylfuran-2-carboxylic acid (Int-3). The product was purified by preparative HPLC to afford Example 27 as a white solid. MS obsd. (ESI⁺) [(M+H)⁺]: 409.1. ¹H NMR (400 MHz, DMSO-d₆) δ ppm: 10.27 (s, 1H), 8.45-8.51 (m, 2H), 7.84 (d, J=2.1 Hz, 1H), 7.77 (d, J=8.7 Hz, 1H), 7.46 (dd, J=8.7, 2.1 Hz, 1H), 2.67 (s, 6H).

### Example 28

### 2,5-dichloro-N-[3-(5-chloro-1,3-benzoxazol-2-yl)-1-bicyclo[1.1.1]pentanyl]benzamide

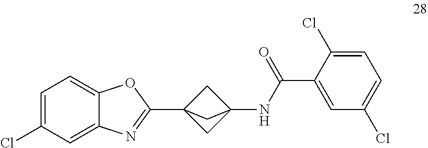 [SMILES: O=C(NC12CC(c3nc4cc(Cl)ccc4o3)(C1)C2)c1cc(Cl)ccc1Cl]

The title compound was prepared in analogy to the procedure described for the preparation of Example 1, by using 2,5-dichlorobenzoic acid instead of 5-methylsulfonylfuran-2-carboxylic acid (Int-3). The product was purified by preparative HPLC to afford Example 28 as a white solid. MS obsd. (ESI⁺) [(M+H)⁺]: 407.0. ¹H NMR (400 MHz, DMSO-d₆) δ ppm: 9.34 (s, 1H), 7.84 (d, J=2.1 Hz, 1H), 7.77 (d, J=8.7 Hz, 1H), 7.52-7.60 (m, 3H), 7.45 (dd, J=8.7, 2.2 Hz, 1H), 2.59 (s, 6H).

### Example 29

### N-[3-(5-chloro-1,3-benzoxazol-2-yl)-1-bicyclo[1.1.1]pentanyl]-3-(methylsulfonimidoyl)benzamide

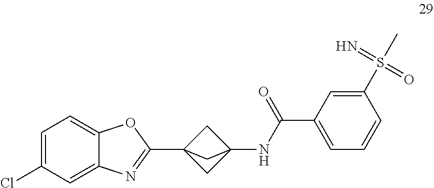 [SMILES: CS(=N)(=O)c1cccc(C(=O)NC23CC(c4nc5cc(Cl)ccc5o4)(C2)C3)c1]

The title compound was prepared in analogy to the procedure described for the preparation of Example 11, by using 3-methylsulfanylbenzoic acid instead of 5-cyclopropylsulfanylfuran-2-carboxylic acid (Int-4). The product was purified by silica gel column to afford Example 29 as a white solid. MS obsd. (ESI⁺) [(M+H)⁺]: 416.2. ¹H NMR (400 MHz, DMSO-d₆) δ ppm: 9.52 (s, 1 H), 8.42 (t, J=1.6 Hz, 1H), 8.07-8.13 (m, 2H), 7.84 (d, J=2.2 Hz, 1H), 7.77 (d, J=8.7 Hz, 1 H), 7.72 (t, J=7.8 Hz, 1H), 7.46 (dd, J=8.7, 2.2 Hz, 1H), 4.34 (s, 1H), 3.09-3.12 (m, 3H), 2.63 (s, 6H).

### Example 30

### N-[3-(5-chloro-1,3-benzoxazol-2-yl)-1-bicyclo[1.1.1]pentanyl]-2,2-dioxo-2lambda6-thiaspiro[3.3]heptane-6-carboxamide

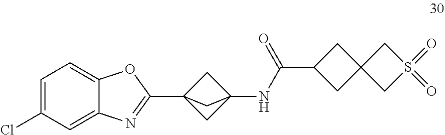 [SMILES: O=C(NC12CC(c3nc4cc(Cl)ccc4o3)(C1)C2)C1CC2(C1)CS(=O)(=O)C2]

The title compound was prepared in analogy to the procedure described for the preparation of Example 1, by using 2,2-dioxo-2lambda6-thiaspiro[3.3]heptane-6-carboxylic acid instead of 5-methylsulfonylfuran-2-carboxylic acid (Int-3). The product was purified by preparative HPLC to afford Example 30 as a white solid. MS obsd. (ESI⁺) [(M+H)⁺]: 406.9. ¹H NMR (400 MHz, DMSO-d₆) δ ppm: 8.60 (s, 1H), 7.81 (d, J=2.2 Hz, 1H), 7.74 (d, J=8.7 Hz, 1H), 7.44 (dd, J=8.7, 2.1 Hz, 1H), 4.08-4.27 (m, 4H), 2.92 (quin, J=8.4 Hz, 1H), 2.45-2.49 (m, 6H), 2.34-2.44 (m, 4H).

### Example 31

### 5-(butanoylsulfamoyl)-N-[3-(5-chloro-1,3-benzoxazol-2-yl)-1-bicyclo[1.1.1]pentanyl]furan-2-carboxamide

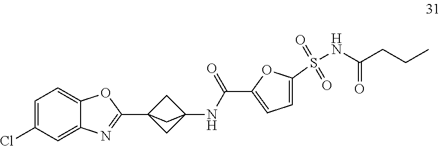 [SMILES: CCCC(=O)NS(=O)(=O)c1ccc(C(=O)NC23CC(c4nc5cc(Cl)ccc5o4)(C2)C3)o1]

The title compound was prepared according to the following scheme:

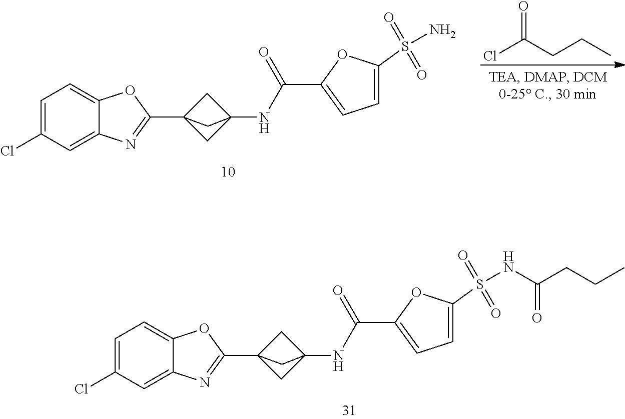

To a solution of N-[3-(5-chloro-1,3-benzoxazol-2-yl)-1-bicyclo[1.1.1]pentanyl]-5-sulfamoyl-furan-2-carboxamide (Example 10, 100 mg, 0.25 mmol), DMAP (1 mg, 8.19 μmol) and TEA (49.6 mg, 0.49 mmol) in DCM (5 mL) was added butyryl chloride (39.2 mg, 0.37 mmol) dropwise at 0° C. After being stirred at room temperature for 30 min, the reaction solution was diluted with DCM (15 mL) and washed with HCl (1 M, 10 mL). The organic layer was dried over Na₂SO₄ and purified by flash column (eluting with DCM/MeOH=50/1 to 30/1) to afford 5-(butanoylsulfamoyl)-N-[3-(5-chloro-1,3-benzoxazol-2-yl)-1-bicyclo[1.1.1]pentanyl]furan-2-carboxamide as a white solid (Example 31, 52 mg, 43.9%). MS obsd. (ESI⁺) [(M+H)⁺]: 478.6. ¹H NMR (400 MHz, DMSO-d₆) δ ppm: 12.48 (br s, 1H), 9.46 (s, 1H), 7.83 (d, J=2.0 Hz, 1H), 7.76 (d, J=8.7 Hz, 1H), 7.45 (dd, J=8.7, 2.1 Hz, 1H), 7.38 (d, J=3.8 Hz, 1H), 7.31 (d, J=3.7 Hz, 1H), 2.60 (s, 6H), 2.25 (t, J=7.2 Hz, 2H), 1.46 (sxt, J=7.4 Hz, 2H), 0.80 (t, J=7.4 Hz, 3H).

### Example 32

### N-[3-(6-chloro-1,3-benzoxazol-2-yl)-1-bicyclo[1.1.1]pentanyl]-5-methylsulfonyl-furan-2-carboxamide

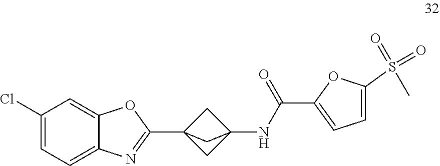 [SMILES: CS(=O)(=O)c1ccc(C(=O)NC23CC(c4nc5ccc(Cl)cc5o4)(C2)C3)o1]

The title compound was prepared in analogy to the procedure described for the preparation of Example 1, by using 2-amino-5-chloro-phenol instead of 2-amino-4-chloro-phenol. The product was purified by preparative HPLC to afford Example 32 as a white solid. MS obsd. (ESI⁺) [(M+H)⁺]: 407.1. ¹H NMR (400 MHz, CDCl₃) δ ppm: 7.60 (d, J=8.44 Hz, 1H), 7.52 (d, J=1.83 Hz, 1H), 7.32 (dd, J=1.96, 8.44 Hz, 1H), 7.21-7.25 (m, 2H), 7.05 (s, 1H), 3.22 (s, 3 H), 2.75 (s, 6H).

### Example 33

### N-[3-(6-chloro-1,3-benzoxazol-2-yl)-1-bicyclo[1.1.1]pentanyl]-5-cyclopropylsulfonyl-furan-2-carboxamide

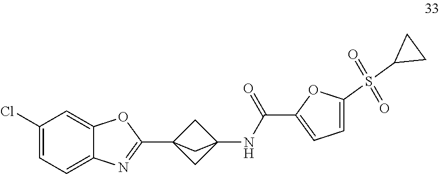 [SMILES: O=C(NC12CC(c3nc4ccc(Cl)cc4o3)(C1)C2)c1ccc(S(=O)(=O)C2CC2)o1]

The title compound was prepared in analogy to the procedure described for the preparation of Example 1, by using 2-amino-5-chloro-phenol instead of 2-amino-4-chloro-phenol and 5-cyclopropylsulfonylfuran-2-carboxylic acid (Int-6) instead of 5-methylsulfonylfuran-2-carboxylic acid (Int-3). The product was purified by preparative HPLC to afford Example 33 as a white solid. MS obsd. (ESI⁺) [(M+H)⁺]: 433.0. ¹H NMR (400 MHz, DMSO-d₆) δ ppm: 9.54 (br s, 1H), 7.94 (d, J=1.71 Hz, 1H), 7.73 (d, J=8.68 Hz, 1H), 7.43 (dd, J=2.02, 8.50 Hz, 1H), 7.39 (d, J=3.67 Hz, 1H), 7.32 (d, J=3.79 Hz, 1H), 2.91-3.02 (m, 1H), 2.61 (s, 6H), 1.20-1.28 (m, 2H), 1.12-1.20 (m, 2H).

### Example 34

### N-[3-(6-chloro-1,3-benzoxazol-2-yl)-1-bicyclo[1.1.1]pentanyl]-5-(cyclopropylmethylsulfonyl)furan-2-carboxamide

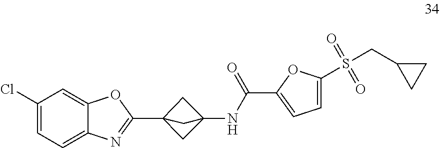 [SMILES: O=C(NC12CC(c3nc4ccc(Cl)cc4o3)(C1)C2)c1ccc(S(=O)(=O)CC2CC2)o1]

The title compound was prepared in analogy to the procedure described for the preparation of Example 1, by using 2-amino-5-chloro-phenol instead of 2-amino-4-chloro-phenol and 5-(cyclopropylmethylsulfonyl)furan-2-carboxylic acid (Int-9) instead of 5-methylsulfonylfuran-2-carboxylic acid (Int-3). The product was purified by preparative HPLC to afford Example 34 as a white solid. MS obsd. (ESI⁺) [(M+H)⁺]: 447.0. ¹H NMR (400 MHz, CD₃OD) δ ppm: 7.69 (d, J=1.96 Hz, 1H), 7.63 (d, J=8.56 Hz, 1H), 7.39 (dd, J=1.96, 8.56 Hz, 1H), 7.34 (d, J=3.67 Hz, 1H), 7.27 (d, J=3.67 Hz, 1H), 3.33 (d, J=7.34 Hz, 2H), 2.69 (s, 6H), 1.00-1.11 (m, 1 H), 0.55-0.61 (m, 2H), 0.16-0.22 (m, 2H).

### Example 35

### N-[3-(6-chloro-1,3-benzoxazol-2-yl)-1-bicyclo[1.1.1]pentanyl]-5-(methylsulfonimidoyl)furan-2-carboxamide

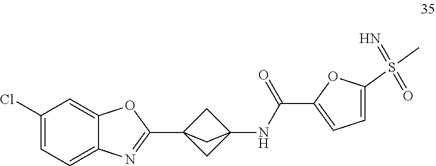 [SMILES: CS(=N)(=O)c1ccc(C(=O)NC23CC(c4nc5ccc(Cl)cc5o4)(C2)C3)o1]

The title compound was prepared according to the following scheme:

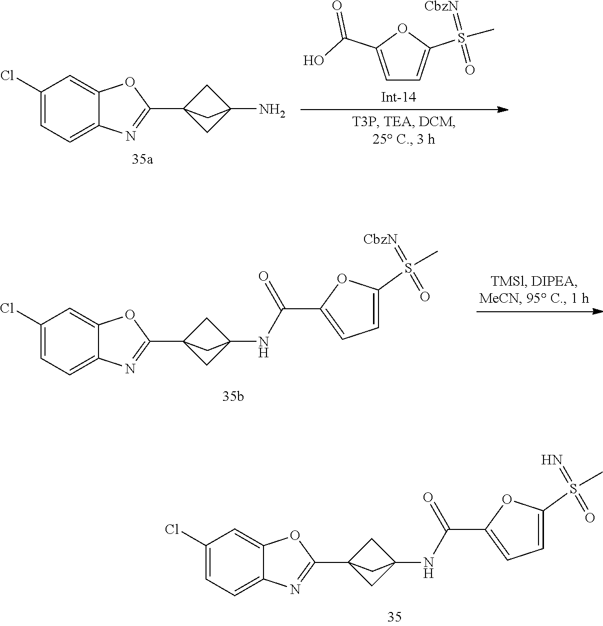

Step 1: Preparation of benzyl N-[[4-[[1-(6-chloro-1,3-benzoxazol-2-yl)-3-bicyclo[1.1.1]pentanyl]carbamoyl]-2-furyl]-methyl-oxo-λ\hat{ }{6}-sulfanylidene]carbamate (35b)

To a solution of 5-(N-benzyloxycarbonyl-S-methyl-sulfonimidoyl)furan-2-carboxylic acid (Int-14, 100 mg, 0.31 mmol) in DCM (5 mL) was added 2-(2-azabicyclo[1.1.1]pentan-3-yl)-6-chloro-1,3-benzoxazole (35a, 81.89 mg, 0.37 mmol, prepared in analogy to the procedure described for the preparation of 1a, by using 2-amino-5-chloro-phenol instead of 2-amino-4-chloro-phenol). Then T₃P (295.23 mg, 0.93 mmol) and triethylamine (93.72 mg, 0.93 mmol) were added. After being stirred at 25° C. for 3 h, the reaction was quenched with water and extracted with DCM (15 mL×3). The combined organic layer was washed with water and brine, dried over MgSO₄ and concentrated in vacuo to give benzyl N-[[4-[[1-(6-chloro-1,3-benzoxazol-2-yl)-3-bicyclo[1.1.1]pentanyl]carbamoyl]-2-furyl]-methyl-oxo-λ\hat{ }{6}-sulfanylidene]carbamate as a yellow oil (35b, 150 mg, 89.8%), which was used for the next step without purification. MS obsd. (ESI⁺) [(M+H)⁺]: 540.1.

Step 2: Preparation of N-[3-(6-chloro-1,3-benzoxazol-2-yl)-3-bicyclo[1.1.1]pentanyl]-5-(methylsulfonimidoyl)furan-2-carboxamide (Example 35)

To a solution of benzyl N-[[5-[[1-(6-chloro-1,3-benzoxazol-2-yl)-3-bicyclo[1.1.1]pentanyl]carbamoyl]-2-furyl]-methyl-oxo-λ\hat{ }{6}-sulfanylidene]carbamate (35b, 50 mg, 0.09 mmol) in acetonitrile (3 mL) were added TMSI (277.91 mg, 1.39 mmol) and DIPEA (179.46 mg, 1.39 mmol). After being stirred at 95° C. for 1 h, the reaction mixture was concentrated in vacuo. The residue was purified by preparative HPLC to give N-[3-(6-chloro-1,3-benzoxazol-2-yl)-3-bicyclo[1.1.1]pentanyl]-5-(methylsulfonimidoyl)furan-2-carboxamide as a brown solid (Example 35, 12.5 mg, 31.7%). MS obsd. (ESI⁺) [(M+H)⁺]: 406.0. ¹H NMR (400 MHz, DMSO-d₆) δ ppm: 9.45 (s, 1H), 7.94 (d, J=1.96 Hz, 1H), 7.73 (d, J=8.56 Hz, 1H), 7.43 (dd, J=2.02, 8.50 Hz, 1H), 7.23 (d, J=3.55 Hz, 1H), 7.16 (d, J=3.67 Hz, 1H), 4.89 (br s, 1H), 3.20 (s, 3H), 2.60 (s, 6H).

### Example 36

### N-[3-(6-chloro-1,3-benzoxazol-2-yl)-1-bicyclo[1.1.1]pentanyl]-3-methylsulfonyl-benzamide

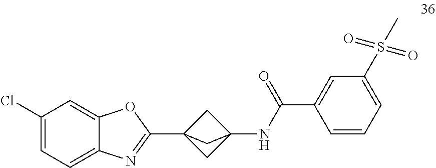 [SMILES: CS(=O)(=O)c1cccc(C(=O)NC23CC(c4nc5ccc(Cl)cc5o4)(C2)C3)c1]

The title compound was prepared in analogy to the procedure described for the preparation of Example 1, by using 2-amino-5-chloro-phenol instead of 2-amino-4-chloro-phenol and 3-methylsulfonylbenzoic acid instead of 5-methylsulfonylfuran-2-carboxylic acid (Int-3). The product was purified by preparative HPLC to afford Example 36 as a white solid. MS obsd. (ESI⁺) [(M+H)⁺]: 417.0. ¹H NMR (400 MHz, DMSO-d₆) δ ppm: 9.56 (s, 1H), 8.41 (t, J=1.71 Hz, 1H), 8.20 (td, J=1.28, 7.95 Hz, 1H), 8.07-8.12 (m, 1H), 7.94 (d, J=1.96 Hz, 1H), 7.70-7.83 (m, 2H), 7.44 (dd, J=1.96, 8.56 Hz, 1H), 3.27 (s, 3H), 2.63 (s, 6H).

### Example 37

### N-[3-(6-chloro-1,3-benzoxazol-2-yl)-1-bicyclo[1.1.1]pentanyl]-2-(trifluoromethyl)pyridine-4-carboxamide

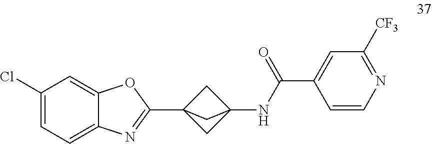 [SMILES: O=C(NC12CC(c3nc4ccc(Cl)cc4o3)(C1)C2)c1ccnc(C(F)(F)F)c1]

The title compound was prepared in analogy to the procedure described for the preparation of Example 1, by using 2-amino-5-chloro-phenol instead of 2-amino-4-chloro-phenol and 2-(trifluoromethyl)pyridine-4-carboxylic acid instead of 5-methylsulfonylfuran-2-carboxylic acid (Int-3). The product was purified by preparative HPLC to afford Example 37 as an off-white solid. MS obsd. (ESI⁺) [(M+H)⁺]: 408.0. ¹H NMR (400 MHz, CD₃OD) δ ppm: 8.87 (d, J=5.14 Hz, 1H), 8.19 (s, 1H), 8.01 (dd, J=1.34, 5.01 Hz, 1H), 7.70 (d, J=1.96 Hz, 1H), 7.63 (d, J=8.56 Hz, 1H), 7.39 (dd, J=1.96, 8.56 Hz, 1H), 2.72 (s, 6H).

### Example 38

### N-[3-(6-chloro-1,3-benzoxazol-2-yl)-1-bicyclo[1.1.1]pentanyl]-2-cyclopropylsulfonyl-pyridine-4-carboxamide

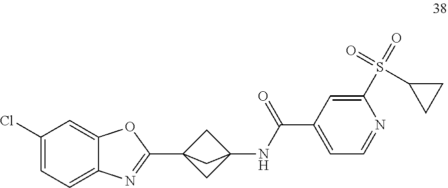 [SMILES: O=C(NC12CC(c3nc4ccc(Cl)cc4o3)(C1)C2)c1ccnc(S(=O)(=O)C2CC2)c1]

The title compound was prepared in analogy to the procedure described for the preparation of Example 1, by using 2-amino-5-chloro-phenol instead of 2-amino-4-chloro-phenol and 2-cyclopropylsulfonylpyridine-4-carboxylic acid (Int-13) instead of 5-methylsulfonylfuran-2-carboxylic acid (Int-3). The product was purified by preparative HPLC to afford Example 38 as a white solid. MS obsd. (ESI⁺) [(M+H)⁺]: 444.0. ¹H NMR (400 MHz, DMSO-d₆) δ ppm: 9.85 (br s, 1H), 8.99 (br d, J=4.3 Hz, 1H), 8.40 (br s, 1H), 8.11 (br d, J=3.8 Hz, 1H), 7.94 (br s, 1H), 7.73 (br d, J=8.3 Hz, 1H), 7.44 (br d, J=8.7 Hz, 1H), 3.00 (br s, 1H), 2.59-2.69 (m, 6H), 1.15 (br s, 4H).

### Example 39

### N-[3-(6-chloro-1,3-benzoxazol-2-yl)-1-bicyclo[1.1.1]pentanyl]-5-(methylsulfonylmethyl)furan-2-carboxamide

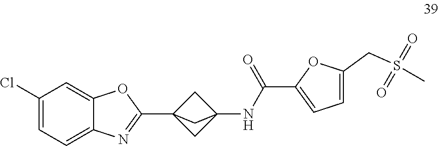 [SMILES: CS(=O)(=O)Cc1ccc(C(=O)NC23CC(c4nc5ccc(Cl)cc5o4)(C2)C3)o1]

The title compound was prepared in analogy to the procedure described for the preparation of Example 1, by using 2-amino-5-chloro-phenol instead of 2-amino-4-chloro-phenol and 5-(methylsulfonylmethyl)furan-2-carboxylic acid instead of 5-methylsulfonylfuran-2-carboxylic acid (Int-3). The product was purified by preparative HPLC to afford Example 39 as a white solid. MS obsd. (ESI⁺) [(M+H)⁺]: 421.3. ¹H NMR (400 MHz, DMSO-d₆) δ ppm: 9.13 (s, 1H), 7.92 (d, J=2.0 Hz, 1H), 7.72 (d, J=8.6 Hz, 1H), 7.43 (dd, J=8.6, 2.0 Hz, 1H), 7.18 (d, J=3.4 Hz, 1H), 6.68 (d, J=3.5 Hz, 1H), 4.72 (s, 2H), 3.06 (s, 3H), 2.59 (s, 6H).

### Example 40

### N-[3-(6-chloro-1,3-benzoxazol-2-yl)-1-bicyclo[1.1.1]pentanyl]-5-[(methylsulfonimidoyl)methyl]furan-2-carboxamide

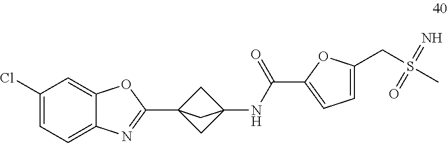 [SMILES: CS(=N)(=O)Cc1ccc(C(=O)NC23CC(c4nc5ccc(Cl)cc5o4)(C2)C3)o1]

The title compound was prepared in analogy to the procedure described for the preparation of Example 17, by using 2-amino-5-chloro-phenol instead of 2-amino-4-chloro-phenol. The product was purified by preparative HPLC to afford Example 40 as a white solid. MS obsd. (ESI⁺) [(M+H)⁺]: 420.0. ¹H NMR (400 MHz, DMSO-d₆) δ ppm: 8.97 (s, 1H), 7.93 (d, J=1.83 Hz, 1H), 7.72 (d, J=8.56 Hz, 1H), 7.43 (dd, J=1.96, 8.56 Hz, 1H), 7.12 (d, J=3.42 Hz, 1H), 6.62 (d, J=3.42 Hz, 1H), 4.48-4.63 (m, 2H), 2.93 (s, 3H), 2.58 (s, 6H).

### Example 41

### N-[3-(6-chlorooxazolo[5,4-b]pyridin-2-yl)-1-bicyclo[1.1.1]pentanyl]-5-(methylsulfonylmethyl)furan-2-carboxamide

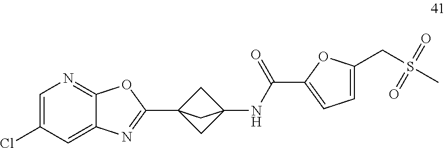 [SMILES: CS(=O)(=O)Cc1ccc(C(=O)NC23CC(c4nc5cc(Cl)cnc5o4)(C2)C3)o1]

The title compound was prepared according to the following scheme:

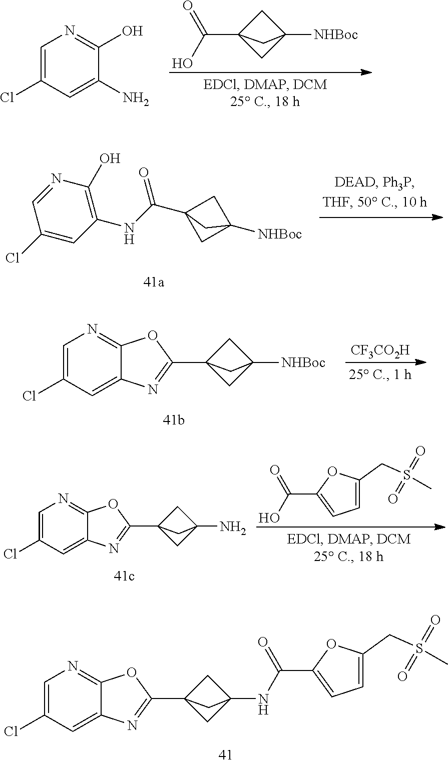

Step 1: Preparation of tert-butyl N-[3-[(5-chloro-2-hydroxy-3-pyridyl)carbamoyl]-1-bicyclo[1.1.1]pentanyl]carbamate (41a)

To a mixture of 3-amino-5-chloropyridin-2-ol (900 mg, 6.23 mmol) and 3-((tert-butoxycarbonyl)amino)bicyclo[1.1.1]pentane-1-carboxylic acid (1.56 g, 6.85 mmol) in DCM (15 mL) were added EDCI (1.79 g, 9.34 mmol) and DMAP (1.14 g, 9.34 mmol). Then the solution was stirred at room temperature for 18 h. The resulting mixture was concentrated in vacuum and the residue was triturated in EtOAc (150 mL). The solid was collected by filtration and washed with saturated aqueous NaHCO3 (150 mL×2), water (150 mL×3). The filter cake was dried in vacuum to afford tert-butyl N-[3-[(5-chloro-2-hydroxy-3-pyridyl)carbamoyl]-1-bicyclo[1.1.1]pentanyl]carbamate as a light gray solid (41a, 2.1 g, 95.3%). MS obsd. (ESI⁺) [(M+H)⁺]: 354.2.

Step 2: Preparation of tert-butyl N-[3-(6-chlorooxazolo[5,4-b]pyridin-2-yl)-1-bicyclo[1.1.1]pentanyl]carbamate (41b)

To a solution of tert-butyl (3-((5-chloro-2-hydroxypyridin-3-yl)carbamoyl)bicyclo[1.1.1]pentan-1-yl)carbamate (41a, 940 mg, 2.66 mmol) and Ph₃P (1.39 g, 5.31 mmol) in THF (30 mL) was added DEAD (694 mg, 631 μl, 3.99 mmol) dropwise at 0° C. After being stirred at room temperature for 0.5 h, the reaction was heated with stirring at 50° C. for 10 h. The reaction solution was concentrated in vacuum and the residue was purified by silica gel column eluted with PE/EtOAc=3/1 to afford tert-butyl (3-(6-chlorooxazolo[5,4-b]pyridin-2-yl)bicyclo[1.1.1]pentan-1-yl)carbamate as a white solid (41b, 809 mg, 86.1%).

Step 3: Preparation of 3-(6-chlorooxazolo[5,4-b]pyridin-2-yl)bicyclo[1.1.1]pentan-1-amine (41c)

A mixture of tert-butyl (3-(6-chlorooxazolo[5,4-b]pyridin-2-yl)bicyclo[1.1.1]pentan-1-yl)carbamate (41b, 700 mg, 2.08 mmol) in TFA (6 mL) was stirred at 25° C. for 1 h. The reaction solution was concentrated in vacuum and the residue was basified by saturated aqueous NaHCO₃ to pH>7. The resulting mixture was extracted with DCM (30 mL×3), dried over Na₂SO₄ and concentrated in vacuum. The residue was purified by silica gel column eluted with DCM/MeOH=13/1 to afford 3-(6-chlorooxazolo[5,4-b]pyridin-2-yl)bicyclo[1.1.1]pentan-1-amine as a white solid (41c, 408 mg, 83%). MS obsd. (ESI⁺) [(M+H)⁺]: 235.9.

Step 4: Preparation of N-[3-(6-chlorooxazolo[5,4-b]pyridin-2-yl)-1-bicyclo[1.1.1]pentanyl]-5-(methylsulfonylmethyl)furan-2-carboxamide (Example 41)

To a mixture of 3-(6-chlorooxazolo[5,4-b]pyridin-2-yl)bicyclo[1.1.1]pentan-1-amine (41c, 50 mg) and 5-(methylsulfonylmethyl)furan-2-carboxylic acid (47.7 mg, 0.233 mmol) in DCM (2 mL) were added DMAP (38.9 mg, 0.318 mmol) and EDCI (61 mg, 0.318 mmol). After being stirred at 25° C. for 18 h, the reaction solution was added to the silica gel column directly and eluted with DCM/MeOH=30/1 to afford N-[3-(6-chlorooxazolo[5,4-b]pyridin-2-yl)-1-bicyclo[1.1.1]pentanyl]-5-(methylsulfonylmethyl)furan-2-carboxamide as a white solid (Example 41, 70 mg, 77.4%). MS obsd. (ESI⁺) [(M+H)⁺]: 422.4. ¹H NMR (400 MHz, DMSO-d₆) δ ppm: 9.15 (s, 1H), 8.42 (d, J=4.9 Hz, 2H), 7.18 (d, J=3.5 Hz, 1H), 6.67 (d, J=3.4 Hz, 1 H), 4.71 (s, 2H), 3.05 (s, 3H), 2.61 (s, 6H).

### Example 42

### N-[3-(6-chlorooxazolo[5,4-b]pyridin-2-yl)-1-bicyclo[1.1.1]pentanyl]-2-(trifluoromethyl)pyridine-4-carboxamide

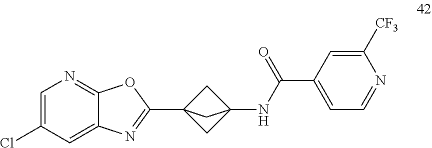 [SMILES: O=C(NC12CC(c3nc4cc(Cl)cnc4o3)(C1)C2)c1ccnc(C(F)(F)F)c1]

The title compound was prepared in analogy to the procedure described for the preparation of Example 41, by using 2-(trifluoromethyl)pyridine-4-carboxylic acid instead of 5-(methylsulfonylmethyl)furan-2-carboxylic acid. The product was purified by preparative HPLC to afford Example 42 as a white solid. MS obsd. (ESI⁺) [(M+H)⁺]: 409.1. ¹H NMR (400 MHz, DMSO-d₆) δ ppm: 9.78 (s, 1H), 8.96 (d, J=5.0 Hz, 1H), 8.43 (q, J=2.3 Hz, 2H), 8.28 (s, 1H), 8.10 (dd, J=5.0, 1.0 Hz, 1H), 2.67 (s, 6H).

### Example 43

### N-[3-(6-chlorooxazolo[4,5-c]pyridin-2-yl)-1-bicyclo[1.1.1]pentanyl]-5-(methylsulfonylmethyl)furan-2-carboxamide

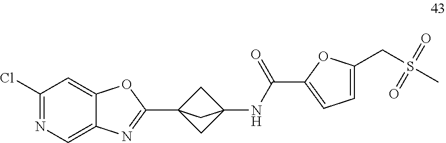 [SMILES: CS(=O)(=O)Cc1ccc(C(=O)NC23CC(c4nc5cnc(Cl)cc5o4)(C2)C3)o1]

The title compound was prepare according to the following scheme:

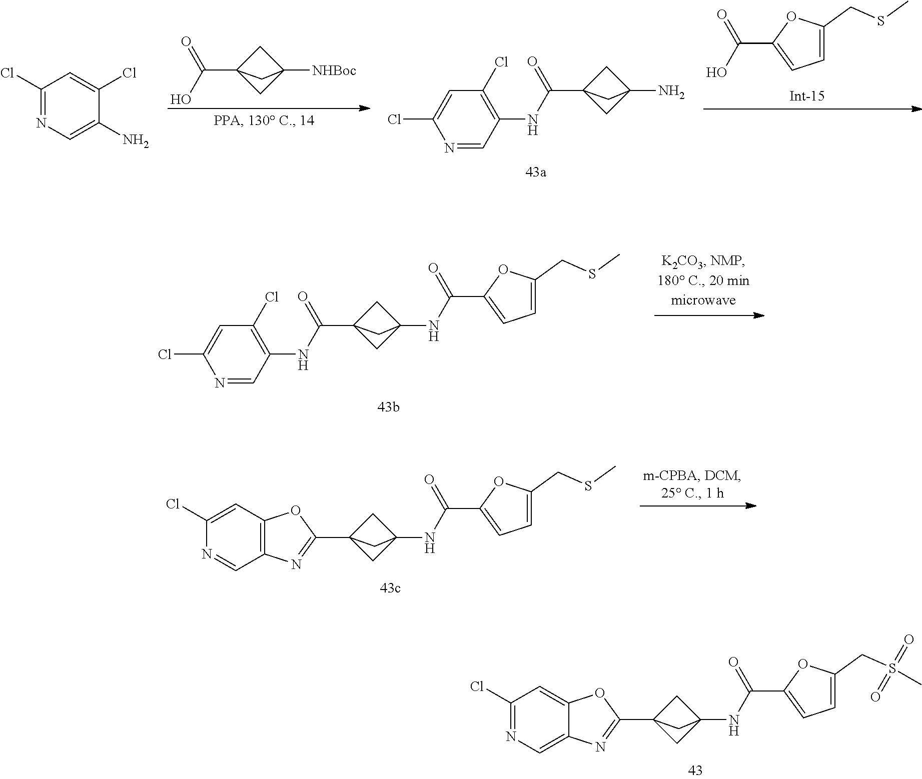

Step 1: Preparation of 3-amino-N-(4,6-dichloro-3-pyridyl)bicyclo[1.1.1]pentane-1-carboxamide (43a)

A mixture of 3-((tert-butoxycarbonyl)amino)bicyclo[1.1.1]pentane-1-carboxylic acid (780.77 mg, 3.44 mmol) and 4,6-dichloropyridin-3-amine (560.0 mg, 3.44 mmol) in PPA (11.6 g, 34.36 mmol) was stirred at 130° C. for 14 h. The reaction was quenched by ice water and basified with ammonia to pH=8 to 10. The resulting mixture was extracted with EtOAc (50 mL×4). The organic layer was washed with brine (50 mL×3), dried over Na₂SO₄ and concentrated. The crude was then purified by flash column chromatography eluted with DCM/MeOH=20/1 to afford 3-amino-N-(4,6-dichloro-3-pyridyl)bicyclo[1.1.1]pentane-1-carboxamide as a yellow solid (43a, 720 mg, 82.61%). MS obsd. (ESI⁺) [(M+H)⁺]: 272.1.

Step 2: Preparation of N-[3-[(4,6-dichloro-3-pyridyl)carbamoyl]-1-bicyclo[1.1.1]pentanyl]-5-(methylsulfanylmethyl)furan-2-carboxamide (43b)

The mixture of 5-(methylsulfanylmethyl)furan-2-carboxylic acid (Int-15, 189.84 mg, 1.1 mmol), HATU (628.76 mg, 1.65 mmol) and DIEA (0.96 mL, 5.51 mmol) in DCM (15 mL) was stirred at 25° C. for 3 min. Then 3-amino-N-(4,6-dichloro-3-pyridyl)bicyclo[1.1.1]pentane-1-carboxamide (43a, 300 mg, 1.1 mmol) was added. After being stirred at 25° C. for further 1 h, the reaction was quenched by water and extracted with DCM (50 mL×3). The combined organic layer was washed with brine (50 mL×3), dried over MgSO₄ and concentrated. The crude was then purified by flash column chromatography eluted with EtOAc/PE=1/2 to afford N-[3-[(4,6-dichloro-3-pyridyl)carbamoyl]-1-bicyclo[1.1.1]pentanyl]-5-(methylsulfanylmethyl)furan-2-carboxamide as a light yellow solid (43b, 370 mg, 78.73%). MS obsd. (ESI⁺) [(M+H)⁺]: 426.0.

Step 3: Preparation of N-[3-(6-chlorooxazolo[4,5-c]pyridin-2-yl)-1-bicyclo[1.1.1]pentanyl]-5-(methylsulfanylmethyl)furan-2-carboxamide (43c)

A mixture of N-[3-[(4,6-dichloro-3-pyridyl)carbamoyl]-1-bicyclo[1.1.1]pentanyl]-5-(methylsulfanylmethyl)furan-2-carboxamide (43c, 94 mg, 0.22 mmol) and K₂CO₃ (45.71 mg, 0.33 mmol) in NMP (2 mL) was stirred at 180° C. for 20 min under microwave irradiation. The resulting mixture was purified by preparative HPLC to afford N-[3-(6-chlorooxazolo[4,5-c]pyridin-2-yl)-1-bicyclo[1.1.1]pentanyl]-5-(methylsulfanylmethyl)furan-2-carboxamide as a light yellow solid (43c, 73.8 mg, 39.0%). MS obsd. (ESI⁺) [(M+H)⁺]: 390.1.

Step 4: Preparation of N-[3-(6-chlorooxazolo[4,5-c]pyridin-2-yl)-1-bicyclo[1.1.1]pentanyl]-5-(methylsulfonylmethyl)furan-2-carboxamide (Example 43)

A mixture of N-[3-(6-chlorooxazolo[4,5-c]pyridin-2-yl)-1-bicyclo[1.1.1]pentanyl]-5-(methylsulfanylmethyl)furan-2-carboxamide (43c, 73.8 mg, 0.19 mmol) and m-CPBA (98.0 mg, 0.57 mmol) in DCM (5 mL) was stirred at 25° C. for 1 h. After being treated with sodium carbonate solution, The organic layer was washed with water and brine, dried over MgSO₄, filtered and concentrated in vacuum. The residue was purified by preparative HPLC to afford N-[3-(6-chlorooxazolo[4,5-c]pyridin-2-yl)-1-bicyclo[1.1.1]pentanyl]-5-(methylsulfonylmethyl)furan-2-carboxamide as a white solid (Example 43, 17.1 mg, 21.3%). MS obsd. (ESI⁺) [(M+H)⁺]: 424.1. ¹H NMR (400 MHz, DMSO-d₆) δ ppm: 9.16 (s, 1H), 8.84 (d, J=0.61 Hz, 1H), 8.07 (d, J=0.73 Hz, 1H), 7.17 (d, J=3.55 Hz, 1H), 6.67 (d, J=3.55 Hz, 1 H), 4.71 (s, 2H), 3.05 (s, 3H), 2.61 (s, 6H).

### Example 44

### N-[3-(7-chloro-1,3-benzoxazol-2-yl)-1-bicyclo[1.1.1]pentanyl]-5-methylsulfonyl-furan-2-carboxamide

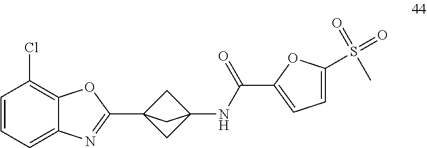 [SMILES: CS(=O)(=O)c1ccc(C(=O)NC23CC(c4nc5cccc(Cl)c5o4)(C2)C3)o1]

The title compound was prepared in analogy to the procedure described for the preparation of Example 1, by using 2-amino-6-chloro-phenol instead of 2-amino-4-chloro-phenol. The product was purified by preparative HPLC to afford Example 44 as a white solid. MS obsd. (ESI⁺) [(M+H)⁺]: 407.0. ¹H NMR (400 MHz, CDCl₃) δ ppm: 7.61 (dd, J=1.10, 7.82 Hz, 1H), 7.33-7.38 (m, 1H), 7.30 (d, J=7.95 Hz, 1H), 7.24-7.27 (m, 2H), 7.10 (s, 1H), 3.24 (s, 3H), 2.80 (s, 6H).

### Example 45

### N-[3-(6-fluoro-1,3-benzoxazol-2-yl)-1-bicyclo[1.1.1]pentanyl]-5-methylsulfonyl-furan-2-carboxamide

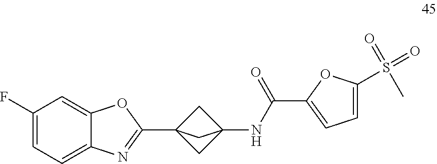 [SMILES: CS(=O)(=O)c1ccc(C(=O)NC23CC(c4nc5ccc(F)cc5o4)(C2)C3)o1]

The title compound was prepared in analogy to the procedure described for the preparation of Example 1, by using 2-amino-5-fluoro-phenol instead of 2-amino-4-chloro-phenol. The product was purified by preparative HPLC to afford Example 45 as a white solid. MS obsd. (ESI⁺) [(M+H)⁺]: 391.1. ¹H NMR (400 MHz, DMSO-d₆) δ ppm: 9.53 (s, 1H), 7.69-7.76 (m, 2 H), 7.39 (d, J=3.67 Hz, 1H), 7.29 (d, J=3.67 Hz, 1H), 7.26 (ddd, J=2.51, 8.83, 9.93 Hz, 1 H), 3.40 (s, 3H), 2.61 (s, 6H).

### Example 46

### N-[4-(5-chloro-1,3-benzoxazol-2-yl)norbornan-1-yl]-5-methylsulfonyl-furan-2-carboxamide

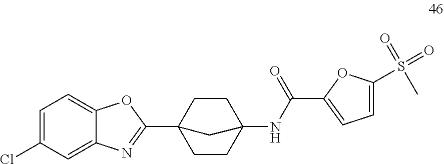 [SMILES: CS(=O)(=O)c1ccc(C(=O)NC23CCC(c4nc5cc(Cl)ccc5o4)(CC2)C3)o1]

The title compound was prepared in analogy to the procedure described for the preparation of Example 1, by using 4-(tert-butoxycarbonylamino)norbomane-1-carboxylic acid instead of 3-(tert-butoxycarbonylamino)bicyclo[1.1.1]pentane-1-carboxylic acid. The product was purified by silica gel column to afford Example 46 as an off-white solid. MS obsd. (ESI⁺) [(M+H)⁺]: 435.2. ¹H NMR (400 MHz, DMSO-d₆) δ ppm: 8.85 (s, 1H), 7.83 (d, J=2.1 Hz, 1H), 7.75 (d, J=8.7 Hz, 1H), 7.31-7.44 (m, 3H), 3.40 (s, 3H), 2.21-2.33 (m, 4H), 1.90-2.15 (m, 6H).

### Example 47

### N-[4-(5-chloro-1,3-benzoxazol-2-yl)-1-bicyclo[2.2.2]octanyl]-5-(trifluoromethyl)furan-2-carboxamide

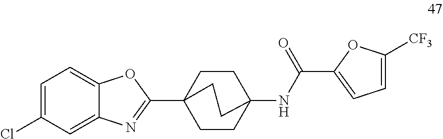 [SMILES: O=C(NC12CCC(c3nc4cc(Cl)ccc4o3)(CC1)CC2)c1ccc(C(F)(F)F)o1]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 5-(trifluoromethyl)furan-2-carboxylic acid instead of 5-methylsulfonylfuran-2-carboxylic acid (Int-3). The product was purified by preparative HPLC to afford Example 47 as a white solid. MS obsd. (ESI⁺) [(M+H)⁺]: 439.1. ¹H NMR (400 MHz, DMSO-d₆) δ ppm: 7.98 (s, 1H), 7.82 (d, J=2.1 Hz, 1H), 7.72 (d, J=8.7 Hz, 1H), 7.31-7.42 (m, 3H), 2.08 (s, 12H).

### Example 48

### N-[4-(5-chloro-1,3-benzoxazol-2-yl)-1-bicyclo[2.2.2]octanyl]-2,2-dioxo-2lambda6-thiaspiro[3.3]heptane-6-carboxamide

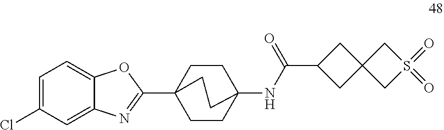 [SMILES: O=C(NC12CCC(c3nc4cc(Cl)ccc4o3)(CC1)CC2)C1CC2(C1)CS(=O)(=O)C2]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 2,2-dioxo-2lambda6-thiaspiro[3.3]heptane-6-carboxylic acid instead of 5-methylsulfonylfuran-2-carboxylic acid (Int-3). The product was purified by silica gel column to afford Example 48 as an off-white solid. MS obsd. (ESI⁺) [(M+H)⁺]: 449.1. ¹H NMR (400 MHz, DMSO-d₆) δ ppm: 7.80 (d, J=2.1 Hz, 1H), 7.71 (d, J=8.7 Hz, 1H), 7.36-7.42 (m, 2H), 4.10-4.25 (m, 4H), 2.95 (quin, J=8.2 Hz, 1H), 2.35 (d, J=8.2 Hz, 4H), 1.88-2.06 (m, 12H).

### Example 49

### N-[4-(5-chloro-1,3-benzoxazol-2-yl)-1-bicyclo[2.2.2]octanyl]-1,1-dioxo-thiolane-2-carboxamide

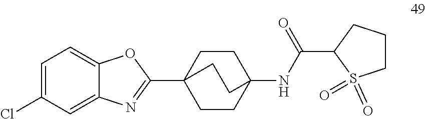 [SMILES: O=C(NC12CCC(c3nc4cc(Cl)ccc4o3)(CC1)CC2)C1CCCS1(=O)=O]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 1,1-dioxothiolane-2-carboxylic acid instead of 5-methylsulfonylfuran-2-carboxylic acid (Int-3). The product was purified by silica gel column to afford Example 49 as an off-white solid. MS obsd. (ESI⁺) [(M+H)⁺]: 422.9. ¹H NMR (400 MHz, DMSO-d₆) δ ppm: 8.05 (br s, 1H), 7.80 (br s, 1H), 7.71 (br d, J=8.4 Hz, 1H), 7.39 (br d, J=8.1 Hz, 1H), 3.83 (br s, 1H), 3.13 (br s, 1H), 2.82-3.04 (m, 1H), 2.29 (br d, J=6.6 Hz, 1H), 2.21-2.30 (m, 4H), 1.85-2.09 (m, 12H).

### Example 50

### N-[4-(5-chloro-1,3-benzoxazol-2-yl)-1-bicyclo[2.2.2]octanyl]-1,1-dioxo-thiolane-3-carboxamide

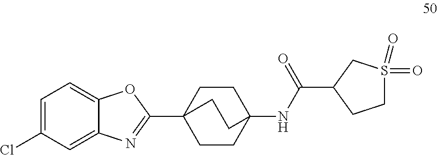 [SMILES: O=C(NC12CCC(c3nc4cc(Cl)ccc4o3)(CC1)CC2)C1CCS(=O)(=O)C1]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 1,1-dioxothiolane-3-carboxylic acid instead of 5-methylsulfonylfuran-2-carboxylic acid (Int-3). The product was purified by preparative HPLC to afford Example 50 as a white solid. MS obsd. (ESI⁺) [(M+H)⁺]: 423.1. ¹H NMR (400 MHz, DMSO-d₆) δ ppm: 7.69-7.82 (m, 3H), 7.39 (dd, J=8.7, 2.1 Hz, 1H), 3.12-3.30 (m, 3H), 2.98-3.09 (m, 2H), 2.19-2.46 (m, 1H), 1.90-2.09 (m, 13H).

### Example 51

### N-[4-(5-chloro-1,3-benzoxazol-2-yl)-1-bicyclo[2.2.2]octanyl]-3-methyl-1,1-dioxo-thiolane-3-carboxamide

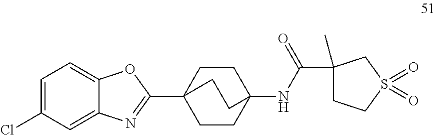 [SMILES: CC1(C(=O)NC23CCC(c4nc5cc(Cl)ccc5o4)(CC2)CC3)CCS(=O)(=O)C1]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 3-methyl-1,1-dioxo-thiolane-3-carboxylic acid instead of 5-methylsulfonylfuran-2-carboxylic acid (Int-3). The product was purified by silica gel column to afford Example 51 as a white solid. MS obsd. (ESI⁺) [(M+H)⁺]: 437.2. ¹H NMR (400 MHz, DMSO-d₆) δ ppm: 7.80 (d, J=2.1 Hz, 1H), 7.71 (d, J=8.7 Hz, 1H), 7.40 (dd, J=8.7, 2.1 Hz, 1H), 7.19 (s, 1H), 3.63 (d, J=13.6 Hz, 1H), 3.21 (dt, J=13.4, 6.7 Hz, 1H), 2.95-3.06 (m, 2H), 2.41-2.48 (m, 1H), 1.94-2.07 (m, 13H), 1.38 (s, 3H).

### Example 52

### N-[4-(5-chloro-1,3-benzoxazol-2-yl)-1-bicyclo[2.2.2]octanyl]-1,1-dioxo-thiane-3-carboxamide

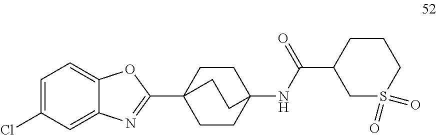 [SMILES: O=C(NC12CCC(c3nc4cc(Cl)ccc4o3)(CC1)CC2)C1CCCS(=O)(=O)C1]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 1,1-dioxothiane-3-carboxylic acid instead of 5-methylsulfonylfuran-2-carboxylic acid (Int-3). The product was purified by silica gel column to afford Example 52 as an off-white solid. MS obsd. (ESI⁺) [(M+H)⁺]: 437.0. ¹H NMR (400 MHz, DMSO-d₆) δ ppm: 7.80 (d, J=2.1 Hz, 1H), 7.66-7.73 (m, 2H), 7.39 (dd, J=8.6, 2.1 Hz, 1H), 3.07-3.18 (m, 2H), 2.97-3.06 (m, 2H), 2.74-2.84 (m, 1H), 1.99-2.08 (m, 7H), 1.86-1.96 (m, 6H), 1.70-1.84 (m, 2H), 1.40-1.64 (m, 1 H).

### Example 53

### N-[4-(5-chloro-1,3-benzoxazol-2-yl)-1-bicyclo[2.2.2]octanyl]-2-(1,1-dioxothiolan-2-yl)acetamide

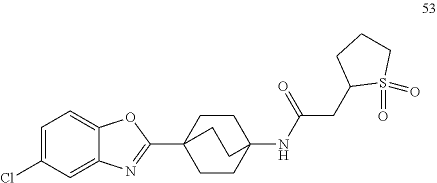 [SMILES: O=C(CC1CCCS1(=O)=O)NC12CCC(c3nc4cc(Cl)ccc4o3)(CC1)CC2]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 2-(1,1-dioxothiolan-2-yl)acetic acid instead of 5-methylsulfonylfuran-2-carboxylic acid (Int-3). The product was purified by silica gel column to afford Example 53 as a white solid. MS obsd. (ESI⁺) [(M+H)⁺]: 436.9. ¹H NMR (400 MHz, DMSO-d₆) δ ppm: 7.80 (d, J=2.1 Hz, 1H), 7.67-7.73 (m, 2H), 7.39 (dd, J=8.7, 2.1 Hz, 1H), 3.21-3.35 (m, 1H), 3.13 (ddd, J=13.4, 8.3, 4.8 Hz, 1 H), 2.96 (dt, J=13.2, 8.4 Hz, 1H), 2.52-2.56 (m, 1H), 2.26 (dd, J=14.8, 9.2 Hz, 2H), 1.90-2.08 (m, 14H), 1.64-1.76 (m, 1H).

### Example 54

### N-[4-(5-chloro-1,3-benzoxazol-2-yl)-1-bicyclo[2.2.2]octanyl]-2-(1,1-dioxothian-3-yl)acetamide

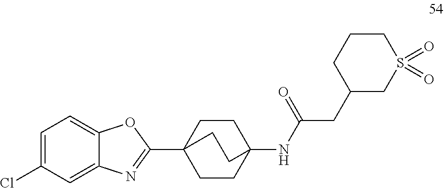 [SMILES: O=C(CC1CCCS(=O)(=O)C1)NC12CCC(c3nc4cc(Cl)ccc4o3)(CC1)CC2]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 2-(1,1-dioxothian-3-yl)acetic acid instead of 5-methylsulfonylfuran-2-carboxylic acid (Int-3). The product was purified by silica gel column to afford Example 54 as a white solid. MS obsd. (ESI⁺) [(M+H)⁺]: 451.0. ¹H NMR (400 MHz, DMSO-d₆) δ ppm: 7.80 (d, J=2.1 Hz, 1H), 7.71 (d, J=8.8 Hz, 1H), 7.55 (s, 1H), 7.39 (dd, J=8.7, 2.2 Hz, 1H), 2.85-3.07 (m, 4H), 2.30 (td, J=7.6, 3.7 Hz, 1H), 2.06-2.12 (m, 2H), 1.91-2.04 (m, 12H), 1.76 (br d, J=9.9 Hz, 1H), 1.68 (br d, J=14.1 Hz, 1H), 1.20-1.30 (m, 2H).

### Example 55

### N-[4-(5-chloro-1,3-benzoxazol-2-yl)-1-bicyclo[2.2.2]octanyl]-2-(1,1-dioxothiazinan-2-yl)acetamide

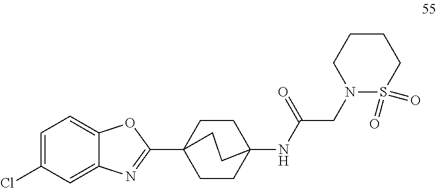 [SMILES: O=C(CN1CCCCS1(=O)=O)NC12CCC(c3nc4cc(Cl)ccc4o3)(CC1)CC2]

The title compound was prepared in analogy to the procedure described for the preparation of Example 1, by using 4-(tert-butoxycarbonylamino)bicyclo[2.2.2]octane-1-carboxylic acid instead of 3-(tert-butoxycarbonylamino)bicyclo[1.1.1]pentane-1-carboxylic acid and 2-(1,1-dioxothiazinan-2-yl)acetic acid instead of 5-methylsulfonylfuran-2-carboxylic acid (Int-3). The product was purified by silica gel column to afford Example 55 as an off-white solid. MS obsd. (ESI⁺) [(M+H)⁺]: 451.9. ¹H NMR (400 MHz, DMSO-d₆) δ ppm: 7.80 (d, J=2.1 Hz, 1H), 7.71 (d, J=8.7 Hz, 1H), 7.38-7.45 (m, 2H), 3.60-3.71 (m, 2H), 3.30-3.37 (m, 2H), 3.12-3.19 (m, 2H), 1.93-2.08 (m, 14H), 1.59 (br s, 2H).

### BIOLOGICAL EXAMPLES

### Example 56

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. 503317). The tube was shaken very gently to re-suspend all cells. 50 μl of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat. 10099141), ng/mL human epidermal growth factor (Gibco, Cat. PHG03I1L), 20 ng/mL dexamethasone (Sigma, Cat. D4902-100 mg), 250 ng/mL human recombinant insulin (Gibco, Cat. 41400045) and 100 U/mL penicillin. HBV virus at 200 genome equivalent (GE) per cell with 4% PEG8000 (Sigma, Cat. P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 1.

TABLE 1
Activity data of compounds of this invention

| Example No. | HBsAg IC₅₀ (μM) | HbeAg IC₅₀ (μM) | CC₅₀ (μM) |
|---|---|---|---|
| Example 1 | 2.170 | 1.772 | >100 |
| Example 2 | 6.447 | 2.139 | >100 |
| Example 2-a | 2.375 | 1.192 | >100 |
| Example 2-b | 3.641 | 1.892 | >100 |
| Example 3 | 8.050 | 3.825 | >100 |
| Example 4 | 2.805 | 1.321 | 79.978 |
| Example 5 | 0.688 | 0.750 | >100 |
| Example 6 | 2.020 | 1.370 | >100 |
| Example 7 | 1.307 | 1.212 | 73.089 |
| Example 8 | 9.035 | 14.803 | >100 |
| Example 9 | 9.643 | 5.367 | >100 |
| Example 10 | 4.437 | 2.668 | >100 |
| Example 11 | 0.823 | 0.594 | >100 |
| Example 12 | 1.515 | 0.891 | >100 |
| Example 12-a | 2.229 | 1.304 | >100 |
| Example 12-b | 2.189 | 1.013 | >100 |
| Example 13 | 0.950 | 0.995 | >100 |
| Example 14 | 5.183 | 3.560 | 95.661 |
| Example 15 | 4.759 | 3.305 | >100 |
| Example 16 | 0.660 | 0.575 | >100 |
| Example 17 | 3.246 | 2.192 | >100 |
| Example 18 | 4.961 | 3.695 | >100 |
| Example 19 | 2.610 | 3.231 | >100 |
| Example 20 | 4.278 | 7.206 | >100 |
| Example 21 | 2.595 | 1.871 | >100 |
| Example 22 | 3.001 | 2.334 | >100 |
| Example 23 | 2.046 | 1.699 | 96.856 |
| Example 24 | 5.227 | 8.823 | >100 |
| Example 25 | 8.710 | 4.360 | >100 |
| Example 26 | 6.291 | 3.668 | 88.243 |
| Example 27 | 3.847 | 1.120 | >100 |
| Example 28 | 9.975 | 10.144 | >100 |
| Example 29 | 3.855 | 3.059 | >100 |
| Example 30 | 9.822 | 7.766 | >100 |
| Example 31 | 3.632 | 4.784 | >100 |
| Example 32 | 5.671 | 3.462 | >100 |
| Example 33 | 0.792 | 0.580 | >100 |
| Example 34 | 3.542 | 2.794 | 95.417 |
| Example 35 | 4.295 | 3.508 | >100 |
| Example 36 | 2.251 | 2.498 | >100 |
| Example 37 | 4.171 | 3.626 | >100 |
| Example 38 | 6.651 | 4.476 | >10 |
| Example 39 | 0.648 | 0.750 | >100 |
| Example 40 | 6.994 | 4.069 | >100 |
| Example 41 | 5.702 | 6.214 | >100 |
| Example 42 | 8.828 | 10.618 | *92.002 |
| Example 43 | 7.621 | 5.551 | >10 |
| Example 44 | 8.932 | 3.104 | >100 |
| Example 45 | 8.633 | 4.475 | >100 |
| Example 46 | 7.146 | 4.481 | 76.587 |
| Example 47 | 7.688 | 3.909 | >100 |
| Example 48 | 1.713 | 1.032 | >100 |
| Example 49 | 2.731 | 1.271 | >100 |
| Example 50 | 4.138 | 5.112 | >100 |
| Example 51 | 9.215 | 4.870 | >100 |
| Example 52 | 2.763 | 1.057 | >100 |
| Example 53 | 2.281 | 1.871 | >100 |
| Example 54 | 3.375 | 3.219 | 43.947 |
| Example 55 | 2.061 | 0.888 | >100 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

1. A compound of the formula (I), 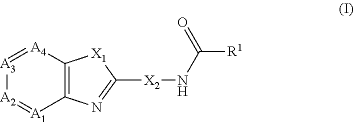
wherein
R¹ is heterocyclyl, heterocyclylC₁₋₆alkyl or phenyl; wherein heterocyclyl, heterocyclylC₁₋₆alkyl and phenyl are unsubstituted or substituted by one or two or three substituents independently selected from halogen, C₁₋₆alkyl, haloC₁₋₆alkyl, C₃₋₇cycloalkyl, heterocyclyl, heterocyclylC₁₋₆alkyl, C₁₋₆alkylsulfonyl, C₁₋₆alkylsulfinyl, C₁₋₆alkylsulfanyl, C₃₋₇cycloalkylsulfinyl, C₃₋₇cycloalkylsulfonyl, C₃₋₇cycloalkylC₁₋₆alkylsulfinyl, C₃₋₇cycloalkylC₁₋₆alkylsulfonyl, aminosulfonyl, C₃₋₇cycloalkylsulfonimidoyl, C₁₋₆alkylsulfonimidoyl, C₃₋₇cycloalkylC₁₋₆alkylsulfonimidoyl, heterocyclylC₁₋₆alkylsulfonyl, heterocyclylC₁₋₆alkylsulfonimidoyl, C₁₋₆alkylsulfonylC₁₋₆alkyl, C₁₋₆alkylsulfonimidoylC₁₋₆alkyl, aminosulfonylC₁₋₆alkyl and C₁₋₆alkylcarbonylaminosulfonyl;
A₁ is N or CR²; wherein R² is H or halogen;
A₂ is N or CR³; wherein R³ is H or halogen;
A₃ is N or CR⁴; wherein R⁴ is H or halogen;
A₄ is N or CR⁵; wherein R⁵ is H or halogen;
X₁ is O;
X₂ is 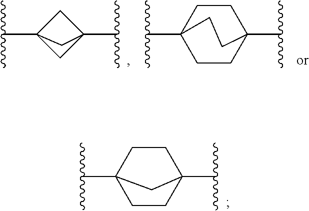
or a pharmaceutically acceptable salt thereof.

2. A compound according to claim 1, wherein
R¹ is furyl, thienyl, pyridyl, pyrimidinyl, pyridazinyl, 2,2-dioxo-2lambda6-thiaspiro[3.3]heptanyl, 1,1-dioxothiolanyl, 1,1-dioxothianyl, 1,1-dioxothiolanylC₁₋₆alkyl, 1,1-dioxothianylC₁₋₆alkyl, 1,1-dioxothiazinanylC₁₋₆alkyl or phenyl; wherein furyl, thienyl, pyridyl, pyrimidinyl, pyridazinyl, 1,1-dioxothiolanyl and phenyl are unsubstituted or substituted by one or two or three substituents independently selected from the group consisting of halogen, C₁₋₆alkyl, haloC₁₋₆alkyl, C₁₋₆alkylsulfonyl, C₁₋₆alkylsulfinyl, C₁₋₆alkylsulfanyl, C₃₋₇cycloalkylsulfinyl, C₃₋₇cycloalkylsulfonyl, C₃₋₇cycloalkylC₁₋₆alkylsulfinyl, C₃₋₇cycloalkylC₁₋₆alkylsulfonyl, aminosulfonyl, C₃₋₇cycloalkylsulfonimidoyl, C₁₋₆alkylsulfonimidoyl, C₃₋₇cycloalkylC₁₋₆alkylsulfonimidoyl, oxetanylC₁₋₆alkylsulfonyl, oxetanylC₁₋₆alkylsulfonimidoyl, C₁₋₆alkylsulfonylC₁₋₆alkyl, C₁₋₆alkylsulfonimidoylC₁₋₆alkyl, aminosulfonylC₁₋₆alkyl and C₁₋₆alkylcarbonylaminosulfonyl;
A₁ is CH;
A₂ is N or CR³; wherein R³ is H or halogen;
A₃ is CR⁴; wherein R⁴ is H or halogen;
A₄ is N or CR⁵; wherein R⁵ is H or halogen;
X₁ is O;
X₂ is 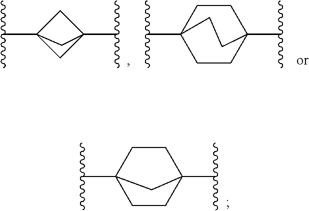
or a pharmaceutically acceptable salt thereof.

3. A compound according to claim 2, wherein
R¹ is s furyl, thienyl, pyridyl, pyrimidinyl, pyridazinyl, 2,2-dioxo-2lambda6-thiaspiro[3.3]heptanyl, 1,1-dioxothiolanyl, 1,1-dioxothianyl, 1,1-dioxothiolanylmethyl, 1,1-dioxothianylmethyl, 1,1-dioxothiazinanylmethyl or phenyl; wherein furyl, pyridyl, pyrimidinyl, pyridazinyl, 1,1-dioxothiolanyl and phenyl are unsubstituted or substituted by one or two or three substituents independently selected from the group consisting of Cl, Br, methyl, CF₃, methylsulfonyl, methylsulfinyl, methylsulfanyl, cyclopropylsulfinyl, cyclopropylsulfonyl, cyclopropylmethylsulfinyl, cyclopropylmethylsulfonyl, aminosulfonyl, cyclopropylsulfonimidoyl, methylsulfonimidoyl, cyclopropylmethylsulfonimidoyl, oxetanylmethylsulfonyl, oxetanylmethylsulfonimidoyl, methylsulfonylmethyl, methylsulfonimidoylmethyl, aminosulfonylmethyl and propylcarbonylaminosulfonyl;
A₁ is CH;
A₂ is N or CR³; wherein R³ is H or Cl;
A₃ is CR⁴; wherein R⁴ is H, F or Cl;
A₄ is N or CR⁵; wherein R⁵ is H or Cl;
X₁ is O;
X₂ is 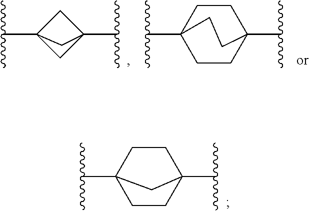
or a pharmaceutically acceptable salt thereof.

4. A compound according to claim 1, or a pharmaceutically acceptable salt thereof, wherein R¹ is furyl; wherein furyl is substituted by one substituent selected from the group consisting of C₃₋₇cycloalkylsulfonyl, C₃₋₇cycloalkylC₁₋₆alkylsulfonyl, C₃₋₇cycloalkylsulfonimidoyl, C₁₋₆alkylsulfonimidoyl, C₃₋₇cycloalkylC₁₋₆alkylsulfonimidoyl and C₁₋₆alkylsulfonylC₁₋₆alkyl.

5. A compound according to claim 4, or a pharmaceutically acceptable salt thereof, wherein R¹ is furyl substituted by one substituent selected from cyclopropylsulfonyl, cyclopropylmethylsulfonyl, cyclopropylsulfonimidoyl, methylsulfonimidoyl, cyclopropylmethylsulfonimidoyl or methylsulfonylmethyl.

6. A compound according to claim 1, or a pharmaceutically acceptable salt thereof, wherein A₂ is CR³; wherein R³ is H or halogen.

7. A compound according to claim 6, or a pharmaceutically acceptable salt thereof, wherein R³ is H or Cl.

8. A compound according to claim 1, or a pharmaceutically acceptable salt thereof, wherein A₃ is CR⁴ and R⁴ is H or halogen.

9. A compound according to claim 8, or a pharmaceutically acceptable salt thereof, wherein R⁴ is H or Cl.

10. A compound according to claim 1, or a pharmaceutically acceptable salt thereof, wherein A₄ is CH.

11. A compound according claim 1, or a pharmaceutically acceptable salt thereof, wherein X₂ is

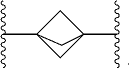 [SMILES: *C12CC(*)(C1)C2]

12. A compound according to claim 1 having the formula (II), 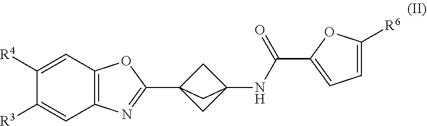
wherein
R³ is H or halogen;
R⁴ is H or halogen;
R⁶ is selected from the group consisting of C₃₋₇cycloalkylsulfonyl, C₃₋₇cycloalkylC₁₋₆alkylsulfonyl, C₃₋₇cycloalkylsulfonimidoyl, C₁₋₆alkylsulfonimidoyl, C₃₋₇cycloalkylC₁₋₆alkylsulfonimidoyl and C₁₋₆alkylsulfonylC₁₋₆alkyl;
or a pharmaceutically acceptable salt thereof.

13. A compound according to claim 12, wherein
R³ is H or Cl;
R⁴ is H or Cl;
R⁶ is selected from the group consisting of cyclopropylsulfonyl, cyclopropylmethylsulfonyl, cyclopropylsulfonimidoyl, methylsulfonimidoyl, cyclopropylmethylsulfonimidoyl and methylsulfonylmethyl;
or a pharmaceutically acceptable salt thereof.

14. A compound according to claim 1, selected from the group consisting of:
N-[3-(5-chloro-1,3-benzoxazol-2-yl)-1-bicyclo[1.1.1]pentanyl]-5-methylsulfonyl-furan-2-carboxamide;
N-[3-(5-chloro-1,3-benzoxazol-2-yl)-1-bicyclo[1.1.1]pentanyl]-5-methylsulfinyl-furan-2-carboxamide;
N-[3-(5-chloro-1,3-benzoxazol-2-yl)-1-bicyclo[1.1.1]pentanyl]-5-[(S)-methylsulfinyl]furan-2-carboxamide;
N-[3-(5-chloro-1,3-benzoxazol-2-yl)-1-bicyclo[1.1.1]pentanyl]-5-[(R)-methylsulfinyl]furan-2-carboxamide;
N-[3-(5-chloro-1,3-benzoxazol-2-yl)-1-bicyclo[1.1.1]pentanyl]-5-methylsulfanyl-furan-2-carboxamide;
N-[3-(5-chloro-1,3-benzoxazol-2-yl)-1-bicyclo[1.1.1]pentanyl]-5-cyclopropylsulfinyl-furan-2-carboxamide;
N-[3-(5-chloro-1,3-benzoxazol-2-yl)-1-bicyclo[1.1.1]pentanyl]-5-cyclopropylsulfonyl-furan-2-carboxamide;
N-[3-(5-chloro-1,3-benzoxazol-2-yl)-1-bicyclo[1.1.1]pentanyl]-5-(cyclopropylmethylsulfinyl)furan-2-carboxamide;
N-[3-(5-chloro-1,3-benzoxazol-2-yl)-1-bicyclo[1.1.1]pentanyl]-5-(cyclopropylmethylsulfonyl)furan-2-carboxamide;
5-bromo-N-[3-(5-chloro-1,3-benzoxazol-2-yl)-1-bicyclo[1.1.1]pentanyl]furan-2-carboxamide;
N-[3-(5-chloro-1,3-benzoxazol-2-yl)-1-bicyclo[1.1.1]pentanyl]-5-methylsulfonyl-thiophene-2-carboxamide;
N-[3-(5-chloro-1,3-benzoxazol-2-yl)-1-bicyclo[1.1.1]pentanyl]-5-sulfamoyl-furan-2-carboxamide;
N-[3-(5-chloro-1,3-benzoxazol-2-yl)-1-bicyclo[1.1.1]pentanyl]-5-(cyclopropylsulfonimidoyl)furan-2-carboxamide;
N-[3-(5-chloro-1,3-benzoxazol-2-yl)-1-bicyclo[1.1.1]pentanyl]-5-(methylsulfonimidoyl)furan-2-carboxamide;
N-[3-(5-chloro-1,3-benzoxazol-2-yl)-1-bicyclo[1.1.1]pentanyl]-5-[(R)-methylsulfonimidoyl]furan-2-carboxamide;
N-[3-(5-chloro-1,3-benzoxazol-2-yl)-1-bicyclo[1.1.1]pentanyl]-5-[(S)-methylsulfonimidoyl]furan-2-carboxamide;
N-[3-(5-chloro-1,3-benzoxazol-2-yl)-1-bicyclo[1.1.1]pentanyl]-5-(cyclopropylmethylsulfonimidoyl)furan-2-carboxamide;
N-[3-(5-chloro-1,3-benzoxazol-2-yl)-1-bicyclo[1.1.1]pentanyl]-5-(oxetan-3-ylmethylsulfonyl)furan-2-carboxamide;
N-[3-(5-chloro-1,3-benzoxazol-2-yl)-1-bicyclo[1.1.1]pentanyl]-5-(oxetan-3-ylmethylsulfonimidoyl)furan-2-carboxamide;
N-[3-(5-chloro-1,3-benzoxazol-2-yl)-1-bicyclo[1.1.1]pentanyl]-5-(methylsulfonylmethyl)furan-2-carboxamide;
N-[3-(5-chloro-1,3-benzoxazol-2-yl)-1-bicyclo[1.1.1]pentanyl]-5-[(methylsulfonimidoyl)methyl]furan-2-carboxamide;
N-[3-(5-chloro-1,3-benzoxazol-2-yl)-1-bicyclo[1.1.1]pentanyl]-5-(sulfamoylmethyl)furan-2-carboxamide;
3-bromo-N-[3-(5-chloro-1,3-benzoxazol-2-yl)-1-bicyclo[1.1.1]pentanyl]benzamide;
4-bromo-N-[3-(5-chloro-1,3-benzoxazol-2-yl)-1-bicyclo[1.1.1]pentanyl]benzamide;
N-[3-(5-chloro-1,3-benzoxazol-2-yl)-1-bicyclo[1.1.1]pentanyl]-3-methylsulfanyl-benzamide;
N-[3-(5-chloro-1,3-benzoxazol-2-yl)-1-bicyclo[1.1.1]pentanyl]-3-methylsulfonyl-benzamide;
N-[3-(5-chloro-1,3-benzoxazol-2-yl)-1-bicyclo[1.1.1]pentanyl]-2-(trifluoromethyl)pyridine-4-carboxamide;
N-[3-(5-chloro-1,3-benzoxazol-2-yl)-1-bicyclo[1.1.1]pentanyl]-5-(trifluoromethyl)pyridine-3-carboxamide;
N-[3-(5-chloro-1,3-benzoxazol-2-yl)-1-bicyclo[1.1.1]pentanyl]-6-(trifluoromethyl)pyridine-3-carboxamide;
N-[3-(5-chloro-1,3-benzoxazol-2-yl)-1-bicyclo[1.1.1]pentanyl]-2-(trifluoromethyl)pyrimidine-5-carboxamide;
N-[3-(5-chloro-1,3-benzoxazol-2-yl)-1-bicyclo[1.1.1]pentanyl]-6-(trifluoromethyl)pyridazine-3-carboxamide;
2,5-dichloro-N-[3-(5-chloro-1,3-benzoxazol-2-yl)-1-bicyclo[1.1.1]pentanyl]benzamide;
N-[3-(5-chloro-1,3-benzoxazol-2-yl)-1-bicyclo[1.1.1]pentanyl]-3-(methylsulfonimidoyl)benzamide;
N-[3-(5-chloro-1,3-benzoxazol-2-yl)-1-bicyclo[1.1.1]pentanyl]-2,2-dioxo-2lambda6-thiaspiro[3.3]heptane-6-carboxamide;
5-(butanoylsulfamoyl)-N-[3-(5-chloro-1,3-benzoxazol-2-yl)-1-bicyclo[1.1.1]pentanyl]furan-2-carboxamide;
N-[3-(6-chloro-1,3-benzoxazol-2-yl)-1-bicyclo[1.1.1]pentanyl]-5-methylsulfonyl-furan-2-carboxamide;
N-[3-(6-chloro-1,3-benzoxazol-2-yl)-1-bicyclo[1.1.1]pentanyl]-5-cyclopropylsulfonyl-furan-2-carboxamide;
N-[3-(6-chloro-1,3-benzoxazol-2-yl)-1-bicyclo[1.1.1]pentanyl]-5-(cyclopropylmethylsulfonyl)furan-2-carboxamide;
N-[3-(6-chloro-1,3-benzoxazol-2-yl)-1-bicyclo[1.1.1]pentanyl]-5-(methylsulfonimidoyl)furan-2-carboxamide;
N-[3-(6-chloro-1,3-benzoxazol-2-yl)-1-bicyclo[1.1.1]pentanyl]-3-methylsulfonyl-benzamide;
N-[3-(6-chloro-1,3-benzoxazol-2-yl)-1-bicyclo[1.1.1]pentanyl]-2-(trifluoromethyl)pyridine-4-carboxamide;
N-[3-(6-chloro-1,3-benzoxazol-2-yl)-1-bicyclo[1.1.1]pentanyl]-2-cyclopropylsulfonyl-pyridine-4-carboxamide;
N-[3-(6-chloro-1,3-benzoxazol-2-yl)-1-bicyclo[1.1.1]pentanyl]-5-(methylsulfonylmethyl)furan-2-carboxamide;
N-[3-(6-chloro-1,3-benzoxazol-2-yl)-1-bicyclo[1.1.1]pentanyl]-5-[(methylsulfonimidoyl)methyl]furan-2-carboxamide;
N-[3-(6-chlorooxazolo[5,4-b]pyridin-2-yl)-1-bicyclo[1.1.1]pentanyl]-5-(methylsulfonylmethyl)furan-2-carboxamide;
N-[3-(6-chlorooxazolo[5,4-b]pyridin-2-yl)-1-bicyclo[1.1.1]pentanyl]-2-(trifluoromethyl)pyridine-4-carboxamide;
N-[3-(6-chlorooxazolo[4,5-c]pyridin-2-yl)-1-bicyclo[1.1.1]pentanyl]-5-(methylsulfonylmethyl)furan-2-carboxamide;
N-[3-(7-chloro-1,3-benzoxazol-2-yl)-1-bicyclo[1.1.1]pentanyl]-5-methylsulfonyl-furan-2-carboxamide;
N-[3-(6-fluoro-1,3-benzoxazol-2-yl)-1-bicyclo[1.1.1]pentanyl]-5-methylsulfonyl-furan-2-carboxamide;
N-[4-(5-chloro-1,3-benzoxazol-2-yl)norboman-1-yl]-5-methylsulfonyl-furan-2-carboxamide;
N-[4-(5-chloro-1,3-benzoxazol-2-yl)-1-bicyclo[2.2.2]octanyl]-5-(trifluoromethyl)furan-2-carboxamide;
N-[4-(5-chloro-1,3-benzoxazol-2-yl)-1-bicyclo[2.2.2]octanyl]-2,2-dioxo-2lambda6-thiaspiro[3.3]heptane-6-carboxamide;
N-[4-(5-chloro-1,3-benzoxazol-2-yl)-1-bicyclo[2.2.2]octanyl]-1,1-dioxo-thiolane-2-carboxamide;
N-[4-(5-chloro-1,3-benzoxazol-2-yl)-1-bicyclo[2.2.2]octanyl]-1,1-dioxo-thiolane-3-carboxamide;
N-[4-(5-chloro-1,3-benzoxazol-2-yl)-1-bicyclo[2.2.2]octanyl]-3-methyl-1,1-dioxo-thiolane-3-carboxamide;
N-[4-(5-chloro-1,3-benzoxazol-2-yl)-1-bicyclo[2.2.2]octanyl]-1,1-dioxo-thiane-3-carboxamide;
N-[4-(5-chloro-1,3-benzoxazol-2-yl)-1-bicyclo[2.2.2]octanyl]-2-(1,1-dioxothiolan-2-yl)acetamide;
N-[4-(5-chloro-1,3-benzoxazol-2-yl)-1-bicyclo[2.2.2]octanyl]-2-(1,1-dioxothian-3-yl)acetamide; and
N-[4-(5-chloro-1,3-benzoxazol-2-yl)-1-bicyclo[2.2.2]octanyl]-2-(1,1-dioxothiazinan-2-yl)acetamide;
or a pharmaceutically acceptable salt thereof.

15. A compound according to any claim 1 selected from the group consisting of:
N-[3-(5-chloro-1,3-benzoxazol-2-yl)-1-bicyclo[1.1.1]pentanyl]-5-cyclopropylsulfonyl-furan-2-carboxamide;
N-[3-(5-chloro-1,3-benzoxazol-2-yl)-1-bicyclo[1.1.1]pentanyl]-5-(cyclopropylmethylsulfonyl)furan-2-carboxamide;
N-[3-(5-chloro-1,3-benzoxazol-2-yl)-1-bicyclo[1.1.1]pentanyl]-5-(cyclopropylsulfonimidoyl)furan-2-carboxamide;
N-[3-(5-chloro-1,3-benzoxazol-2-yl)-1-bicyclo[1.1.1]pentanyl]-5-(methylsulfonimidoyl)furan-2-carboxamide;
N-[3-(5-chloro-1,3-benzoxazol-2-yl)-1-bicyclo[1.1.1]pentanyl]-5-(cyclopropylmethylsulfonimidoyl)furan-2-carboxamide;
N-[3-(5-chloro-1,3-benzoxazol-2-yl)-1-bicyclo[1.1.1]pentanyl]-5-(methylsulfonylmethyl)furan-2-carboxamide;
N-[3-(6-chloro-1,3-benzoxazol-2-yl)-1-bicyclo[1.1.1]pentanyl]-5-cyclopropylsulfonyl-furan-2-carboxamide; and
N-[3-(6-chloro-1,3-benzoxazol-2-yl)-1-bicyclo[1.1.1]pentanyl]-5-(methylsulfonylmethyl)furan-2-carboxamide;
or a pharmaceutically acceptable salt thereof.

16. A process for the preparation of a compound according to claim 1 comprising at least one of the following steps,
(a) Coupling of a compound of formula (IV), 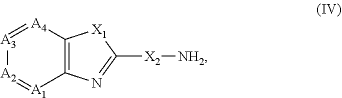
with a compound of formula (V), 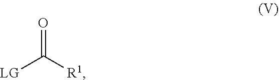
in the presence of a coupling reagent and a base;
(b) Cyclization of a compound of formula (VI-2), 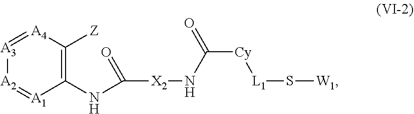
in the presence of a base;
(c) Oxidation of a compound of formula (I-2), 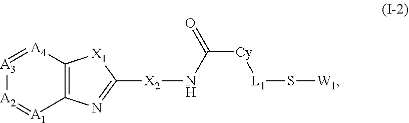
in the presence of an oxidate;
(d) Deprotection of a compound of formula (VII), 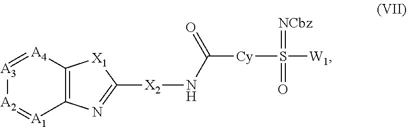
with TMSI, in the presence of a base;
(e) Deprotection of a compound of formula (VIII), 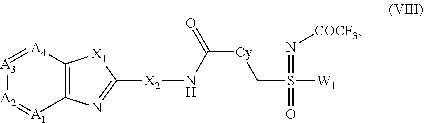
in the presence of a base;
(f) Reaction of a compound of formula (I-7), 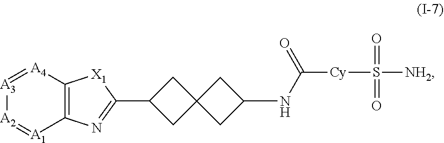
with a halide (IX), LG-W₂ (IX), in the presence of a base;
wherein A₁ to A₄, X₁, X₂ and R¹ are defined as in claim 1; Z is halogen or OH; Cy is furyl or phenyl; LG is OH or halogen; L₁ is —CH₂— or a bond; W₁ is C₁₋₆alkyl, C₃₋₇cycloalkyl, C₃₋₇cycloalkylC₁₋₆alkyl, heterocyclyl or heterocyclylC₁₋₆alkyl; W₂ is C₁₋₆alkylcarbonyl.

17. A compound according to claim 1 for use as therapeutically active substance.

18. A pharmaceutical composition comprising a compound of claim 1 and a therapeutically inert carrier.

19. The use of a compound of claim 1 for the treatment or prophylaxis of HBV infection.

20. The use of a compound of claim 1 for the preparation of a medicament for the treatment of HBV infection.

21. The use of a compound of claim 1 for the inhibition of HBeAg.

22. The use of a compound of claim 1 for the inhibition of HBsAg.

23. The use of a compound of claim 1 for the inhibition of HBV DNA.

24. A compound according to claim 1 for use in the treatment of HBV infection.

25. A method for the treatment of HBV infection, which method comprises administering an effective amount of a compound of claim 1.
